(19) United States
(12) Patent Application Publication
(10) Pub. No.: US 2024/0269206 A1
(43) Pub. Date: Aug. 15, 2024

(54) AMANITA MUSCARIA EXTRACTS AND COMPOUNDS AND THEIR BENEFICIAL AND THERAPEUTIC USE

(71) Applicant: Psyched Wellness Ltd., Toronto (CA)

(72) Inventor: Brian Tancowny, Beaumont (CA)

(21) Appl. No.: 18/683,202

(22) PCT Filed: Aug. 11, 2022
(86) PCT No.: PCT/CA22/51230

§ 371 Date: Feb. 12, 2024

Related U.S. Application Data
(60) Provisional application No. 63/232,144, filed on Aug. 11, 2021.
(60) Provisional application No. 63/232,152, filed on Aug. 11, 2021.
(60) Provisional application No. 63/232,157, filed on Aug. 11, 2021.
(60) Provisional application No. 63/232,160, filed on Aug. 11, 2021.

(51) Int. Cl. A61K 36/07; A61K 9/00; A61K 9/70; A61K 31/341; A61K 31/42; A61K 31/425; A61K 45/06; A61P 25/00; C07D 261/12; C07D 275/03; C07D 307/20

(52) U.S. Cl. CPC A61K 36/07; A61K 9/0014; A61K 9/006; A61K 9/7023; A61K 31/341; A61K 31/42; A61K 31/425; A61K 45/06; A61P 25/00; C07D 261/12; C07D 275/03; C07D 307/20

(57) ABSTRACT

Provided are beneficial and therapeutic Amanita muscaria compositions. In some embodiments, the provided A. muscaria compositions comprise A. muscaria extracts. In some embodiments, the provided A. muscaria compositions comprise one or more compounds from A. muscaria such as muscimol and muscarine, as well as analogs thereof. In some aspects, the provided A. muscaria compositions are used to promote health and wellness, such as to enhance calmness in a subject. In some aspects, the provided A. muscaria compositions are used to treat mental health conditions and physical health conditions in a subject.

### CROSS-REFERENCE

This application claims priority under PCT Art. 8(1) and Rule 4.10 to U.S. Provisional Application Nos. 63/232,144; 63/232,152; 63/232,157; and 63/232,160, all of which were filed Aug. 11, 2021, and are incorporated by reference for all purposes as if fully set forth herein.

### FIELD OF THE INVENTION

The present disclosure relates in some aspects to extracts of Amanita muscaria mushrooms. In some embodiments, the provided A. muscaria extracts comprise a weight ratio of muscimol to ibotenic acid in excess of 1000:1. In some embodiments, the provided A. muscaria extracts are essentially devoid of stizolobinic acid and heavy metal contaminants, including cadmium, arsenic, lead and mercury. In some aspects, the disclosure further relates to isolated compounds of A. muscaria and analogs thereof. In some embodiments, isolated compounds of A. muscaria are provided alone or in combination with additional A. muscaria compounds. In some embodiments, A. muscaria compounds include muscimol, muscarine, ibotenic acid, and analogs thereof. In some aspects, features of the extracts and compounds provide therapeutic benefits in the absence of toxicity. In some embodiments, the provided A. muscaria extracts and compounds are used to improve mammalian health and well-being.

### BACKGROUND OF THE INVENTION

Various strategies are available to use A. muscaria mushrooms, including extracts, compounds, and analogs thereof, to safely and effectively improve health and wellness. For example, strategies are available to provide compositions with increased muscimol to ibotenic acid ratios, such as a w/w ratio, as ibotenic acid can act as a toxin and cause a breadth of side effects ranging from gastrointestinal distress to neurotoxicity. Improved strategies are needed, for example, to provide non-toxic compositions that retain efficacy and improve a variety of ailments and treatment indications. Provided herein are, for example, A. muscaria extracts, compounds, analogs, compositions, methods, and kits to meet these and other needs.

### INCORPORATION BY REFERENCE

Each patent, publication, and non-patent literature cited in the application is hereby incorporated by reference in its entirety as if each was incorporated by reference individually. Reference to any document is not to be construed as an admission that the document referred to or any underlying information in the document is prior art in any jurisdiction, or forms part of the common general knowledge in the art.

### BRIEF SUMMARY OF THE INVENTION

The following presents a simplified summary of some embodiments of the invention in order to provide a basic understanding thereof. This summary is not an extensive overview, nor is it intended to identify every key or critical element of the invention or to delineate the complete scope of the invention. Its sole purpose is to present some exemplary embodiments in a simplified form as a prelude to the more detailed description below.

In some aspects are provided Amanita muscaria extracts, wherein the extract comprises a weight ratio of muscimol to ibotenic acid of at least 1000:1. In some embodiments, the extract comprises a weight ratio of muscimol to ibotenic acid of at least 1500:1. In some embodiments, the extract comprises a weight ratio of muscimol to ibotenic acid of about 1500:1, 1550:1, 1600:1, 1650:1, 1700:1, 1750:1, 1800:1, 1850:1, 1900:1, 1950:1, or 2000:1. In some embodiments, the extract comprises a weight ratio of muscimol to muscarine of at least 40:1. In some embodiments, the extract comprises a weight ratio of muscimol to muscarine of about 40:1, 45:1, 50:1, 55:1, 60:1, 65:1, or 70:1. In some embodiments, the extract comprises less than 30,000 μg/g of muscimol. In some embodiments, the extract comprises less than 550 μg/g of muscarine. In some embodiments, the extract comprises less than 20 μg/g of ibotenic acid. In some embodiments, the extract comprises less than 40 μg/g of stizolobinic acid. In some aspects are provided dietary supplements comprising the disclosed extracts. In some aspects are provided functional foods comprising the disclosed extracts, such as food products. In some embodiments, the functional food is a beverage, such as a ready-to-drink beverage or a beverage powder. In some aspects are provided pharmaceutical compositions comprising the disclosed extracts.

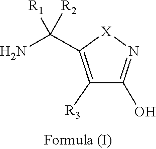

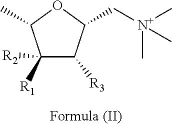

In some aspects are provided compounds of Formula (I), wherein R₁ is H or —CH₃; wherein R₂ is H, —CH₃, —CH₂CH₃, —CH₂CH₂CH₃, —CH(CH₃)₂, —CH(CH₃)(CH₂CH₃), —CH₂CH(CH₃)₂, —C(O)(OCH₃), —C(O)(OCH₂CH₃), —CH₂CH₂SCH₃, phenyl, or benzyl; wherein R₃ is H, —CH₃, —CH₂CH₃, —CH₂CH₃, —CH₂CH₂OH, —CH₂CH₂Cl, F, Br, I, —CF₃, or CN; and wherein X is O or S; or a pharmaceutically acceptable salt, prodrug, hydrate, or solvate thereof. In some aspects are provided compounds of Formula (II), wherein R₁ is H, —OH, F, —O-benzyl, or —O-benzoyl; wherein R₂ is H or —OH; and wherein R₃ is H, —OH, F, or phenyl; or a pharmaceutically acceptable salt, prodrug, hydrate, or solvate thereof.

In some aspects are provided pharmaceutical compositions comprising a compound of Formula (I), a compound of Formula (II), or a compound of Formula (I) and a compound of Formula (II). In some embodiments of the pharmaceutical composition having a compound of Formula (I) and a compound of Formula (II), the compound of Formula (I) and the compound of Formula (II) are present in a weight ratio of at least 40:1. In some embodiments of the pharmaceutical composition having a compound of Formula (I) and a compound of Formula (II), the compound of Formula (I) and the compound of Formula (II) are present in a weight ratio of about 40:1, 45:1, 50:1, 55:1, 60:1, 65:1, or 70:1. In some embodiments of a disclosed pharmaceutical composition, the composition further comprises a weight ratio of muscimol to ibotenic acid of at least 1000:1. In some embodiments of a disclosed pharmaceutical composition, the composition does not comprise stizolobinic acid. In some embodiments of a disclosed pharmaceutical composition, the composition further comprises a pharmaceutically acceptable carrier, diluent, or excipient.

In some aspects are provided extracts, compounds, and pharmaceutical compositions suitable for oral, sublingual, buccal, mucosal, injectable, intranasal, inhalation, or transdermal administration. In some aspects are provided extracts, compounds, and pharmaceutical compositions prepared as a liquid suspension, a liquid solution, a tincture, a tablet, a capsule, an intravenous solution, an injectable form, a topical form, a transdermal delivery form, a cut matrix sublingual or buccal tablet, a sublingual or buccal lozenge, a confectionery product, a vaporizer formulation, an intranasal delivery form, a beverage concentrate or beverage, a syrup, an elixir, an oral thin film, a troche, a caplet, a functional food, an effervescent powder, an herbal or medicinal tea, a nutritional supplement, a nutritional softgel supplement, a nutraceutical powder, and an infused chocolate. In some aspects are provided extracts, compounds, and pharmaceutical compositions in unit dosage form. In some aspects are provided extracts, compounds, and pharmaceutical compositions suitable for oral administration. In some aspects are provided extracts, compounds, and pharmaceutical compositions suitable for sublingual or buccal administration. In some aspects are provided extracts, compounds, and pharmaceutical compositions suitable for topical or transdermal administration. In some aspects are provided extracts, compounds, and pharmaceutical compositions further comprising one or more additional active agents. In some embodiments, the one or more additional active agents is any of an agent useful in treating an anxiety disorder, an agent useful in treating a substance use disorder, an agent useful in treating pain or a pain disorder, an agent useful in treating an inflammatory disease or disorder, an agent useful in treating immune or autoimmune disorders, and a serotonergic agent.

In some aspects are provided methods of modulating neurotransmission comprising administering the disclosed extract, compound, or pharmaceutical composition to a subject, thereby modulating neurotransmission in said subject. In some embodiments, the neurotransmission is one or more of gabaminergic neurotransmission, glutaminergic neurotransmission, and cholinergic neurotransmission. In some aspects are provided methods of treating a health condition, comprising administering to a patient an effective amount of the disclosed extract, compound, or pharmaceutical composition. In some embodiments, the health condition is a mental health disorder. In some embodiments, the mental health disorder is selected from depression, dysthymia, an anxiety and phobia disorders, generalized anxiety disorder, social anxiety disorder, panic disorder, post-traumatic stress disorder, an adjustment disorders, a feeding and eating disorders, binge eating disorder, bulimia, and anorexia nervosa, other binge behaviors, body dysmorphic syndromes, alcoholism, tobacco abuse, drug abuse or dependence disorders, disruptive behavior disorders, impulse control disorders, gaming disorders, gambling disorders, memory loss, dementia of aging, attention deficit hyperactivity disorder, personality disorders, antisocial personality disorder, avoidant personality disorder, borderline personality disorder, histrionic personality disorder, narcissistic personality disorder, obsessive compulsive disorder, paranoid personality disorder, schizoid personality disorder, schizotypal personality disorders, attachment disorders, autism, and dissociative disorders. In some embodiments, the mental health disorder is an anxiety disorder. In some embodiments, the anxiety disorder is any of acute stress disorder, anxiety due to a medical condition, generalized anxiety disorder, panic disorder, panic attack, a phobia, post traumatic stress disorder (PTSD), separation anxiety disorder, social anxiety disorder, substance-induced anxiety disorder, and selective mutism. In some embodiments, the mental health disorder is a substance use disorder. In some embodiments, the substance use disorder is any of alcohol use disorder, Cannabis use disorder, hallucinogen use disorder, inhalant use disorder, opioid use disorder, sedative use disorder, stimulant use disorder, tobacco use disorder, and nicotine use disorder. In some embodiments, the mental health disorder is a behavioral addiction. In some embodiments, the behavioral addiction is selected from gambling disorder, gaming disorder, sexual addiction, compulsive buying disorder, and technology addiction. In some embodiments, the health condition is a sleep disorder. In some embodiments, the sleep disorder is any of an insomnia, a hypersomnia, a parasomnia, and a disorder of sleep-wake schedule.

In some embodiments, the health disorder is a physical health disorder. In some embodiments, the physical health disorder is a pain disorder. In some embodiments, the pain disorder is any of arthritis, allodynia, atypical trigeminal neuralgia, trigeminal neuralgia, somatoform disorder, hypoesthesis, hypealgesia, neuralgia, heuritis, neurogenic pain, analgesia, anesthesia dolorosa, causlagia, sciatic nerve pain disorder, degenerative joint disorder, fibromyalgia, visceral disease, chronic pain disorders, migraine/headache pain, chronic fatigue syndrome, complex regional pain syndrome, neurodystrophy, plantar fasciitis, or pain associated with cancer. In some embodiments, the physical health disorder is a disorder that causes acute inflammation, or that exhibits chronic inflammation as a symptom.

In some embodiments, the physical health disorder is an autoimmune disorder. In some embodiments, the autoimmune disorder is any of acute disseminated encephalomyelitis (ADEM), Addison disease, allergy or hypersensitivity, amyotrophic lateral sclerosis, antiphospholipid antibody syndrome (APS), arthritis, autoimmune hemolysis Anemia, autoimmune hepatitis, autoimmune inner ear disease, autoimmune pancreatitis, bullous pemphigoid, celiac disease, Chagas disease, chronic obstructive pulmonary disease (COPD), type 1 diabetes (T1D), endometriosis, fibromyalgia, goodpasture's syndrome, Graves' disease, Guillain-Barre syndrome (GBS), Hashimoto's thyroiditis, suppurative spondylitis, idiopathic thrombocytopenic purpura, inflammatory bowel disease, interstitial cystitis, lupus, including discoid lupus erythematosus, drug-induced lupus lupus erythematosus, lupus nephritis, neonatal lupus, subacute cutaneous lupus erythematosus, and systemic lupus erythematosus; morphea, multiple hard Keratosis (MS), myasthenia gravis, myopathy, narcolepsy, neuromuscular angina, pemphigus vulgaris, pernicious anemia, primary biliary cirrhosis, recurrent diffuse encephalomyelitis, including polyphasic diffuse encephalomyelitis, rheumatic fever, schizophrenia, scleroderma, Sjogren's syndrome, tendonitis, vasculitis, and vitiligo. In some embodiments, the autoimmune disorder is a systemic autoimmune disorder, including systemic lupus erythematosus (SLE), Sjogren's syndrome, scleroderma, rheumatoid arthritis, and polymyositis. In some embodiments, the autoimmune disorder is a local autoimmune disorder, including those of the endocrine system, including type 1 diabetes, Hashimoto's thyroiditis, and Addison's disease; the cutaneous, including pemphigus vulgaris; the blood, including autoimmune hemolytic anemia; and the nervous system, including multiple sclerosis.

In some aspects are provided methods of using the disclosed extract, compound, or composition to improve health and wellness, comprising administering an effective amount of the extract, compound, or composition to a subject. In some embodiments, the improvement in health and wellness is a reduction in stress. In some embodiments, the improvement in health and wellness is an easing of muscular tension. In some embodiments, the improvement to health and wellness is a promotion of restorative sleep. In some embodiments, the improvement to health and wellness is any of a soothing of the body, a calming of the mind, and a reduction in physical distress. In some embodiments, the improvement to health and wellness includes any one or more of a reduction in feelings of nervousness, “jitters,” nervous tension, or anxiety; a reduction in feelings of malaise, unhappiness, existential angst, ennui, and general discontent; and an increase in feelings of wellbeing, wellness, relaxation, contentment, happiness, openness to experience, and life satisfaction. In some aspects are provided methods of using the disclosed extract, compound, or composition to induce euphoria, comprising administering an effective amount of the extract, compound, or composition to an individual.

The foregoing outlines broadly some pertinent features of certain exemplary embodiments of the disclosure so the detailed description that follows may be better understood and so the present contribution to the art can be more fully appreciated. Additional features of the invention will be described hereinafter which form the subject of the claims of the invention. It should be appreciated by those skilled in the art that the conception and the disclosed specific formulations and methods may be readily utilized as a basis for modifying or designing other formulations and methods for carrying out the same purposes of the disclosure. It should be also realized that such equivalent formulations and methods do not depart from the spirit and scope of the invention as set forth in the claims. Hence, this summary is made with the understanding that it will be considered as a brief and general synopsis of only some of the objects and embodiments herein, is provided solely for the benefit and convenience of the reader, and is not intended to limit in any manner the scope, or range of equivalents, to which the claims are lawfully entitled. It will be appreciated that headings in this disclosure are used only to expedite its review by a reader. They should not be construed as limiting the invention in any manner.

### BRIEF SUMMARY OF THE DRAWINGS

To further clarify various aspects of the invention, a more particular description is made by reference to certain exemplary embodiments illustrated in the figures. It will be appreciated that these figures depict only illustrated embodiments of the invention and should not be considered limiting of its scope. They are merely provided as exemplary illustrations of certain concepts of some embodiments of the invention. Certain aspects of the invention are therefore further described and explained with additional specificity and detail, but still by way of example only, with reference to the accompanying figures in which:

FIG. 1 shows the effects of exemplary A. muscaria extract AME-1 on human microglial clone 3 cell line (HMC3), specifically cytokine production. FIG. 1A shows the production of IL-8 by LPS-activated HMC3 cells exposed to AME-1. FIG. 1B shows the production of IL-6 by LPS-activated HMC3 cells exposed to AME-1. FIG. 1C shows production of IL-8 by polyL:C-induced HMC3 cells exposed to AME-1 (n=3, *=p<0.05, **=p<0.05). FIG. 1D shows production of IL-6 by polyL:C-induced HMC3 cells exposed to AME-1. FIG. 1E shows production of IL-8 by TNF-induced HMC3 cells exposed to AME-1. FIG. 1F shows production of IL-6 by TNF-induced HMC3 cells exposed to AME-1.

FIG. 2 shows preference scores (mean±standard error) for four groups of rats prior to (H) and after (1, 2) place conditioning, wherein two doses of exemplary A. muscaria extract AME-1 (800 mg/kg and 1100 mg/kg) were compared to saline (negative control) and 20 mg/kg cocaine (positive control).

FIG. 3 shows rat gut microvilli expression of Dendritic cell-associated C-type lectin-1 (Dectin-1) in response to 14-day repeated dosing of various doses of exemplary A. muscaria extract AME-1 (500 mg/kg, 800 mg/kg, and 1100 mg/kg) via oral gavage in comparison to a negative control (Vehicle).

FIG. 4 shows rat gut microvilli expression of Toll-like receptor 2 (TLR2) in response to 14-day repeated dosing of various doses of exemplary A. muscaria extract AME-1 (500 mg/kg, 800 mg/kg, and 1100 mg/kg) via oral gavage compared to a negative control (Vehicle).

### DETAILED DESCRIPTION

Various aspects and features of certain some embodiments are summarized above; the following detailed description illustrates further exemplary embodiments in more detail to enable one of skill to practice such embodiments, and to make and use the full scope of the invention.

It will be understood that many modifications, substitutions, changes, and variations in the described examples, embodiments, applications, and details of the invention can be made by one of skill without departing from the spirit of the invention, or the scope of the invention as described in the appended claims, and the general principles defined herein may be applied to a wide range of aspects. Thus, the invention is not intended to be limited to the aspects presented, but is to be accorded the widest scope consistent with the principles and novel features disclosed. The description below is designed to make such embodiments apparent to one of skill, in that the embodiments shall be both readily cognizable and readily creatable without undue experimentation, solely using the teachings herein together with general knowledge of the art.

### A. GENERAL DEFINITIONS AND TERMS

When introducing elements of the disclosure or embodiments thereof, the articles “a,” “an,” “the,” and “said” will mean there are one or more of the elements. Any reference to an element in the singular is not intended to mean “one and only one” unless specifically stated, but rather “one or more”; thus, the term “or” standing alone, unless context demands otherwise, shall mean the same as “and/or.” The terms “comprising,” “including,” “such as,” and “having” are also intended to be inclusive and not exclusive (i.e., there may be other elements in addition to the recited elements). Thus, for example, the terms “including,” “may include,” and “include,” as used herein mean, and are used interchangeably with, the phrase “including but not limited to.” The word “exemplary” is used herein to mean “serving as an example, instance, or illustration.” Any aspect, embodiment, process, or implementation described herein as “exemplary” is therefore not to be construed as necessarily preferred or advantageous over others.

Unless otherwise stated, all measurements, values, ratings, positions, magnitudes, sizes, locations, orientations, configurations, and other specifications that are set forth (either expressly or impliedly) in this specification, including in the figures and in the claims that follow, are approximate, and not exact. They are intended to have a reasonable range that is consistent with the functions to which they relate and with what is customary in the art to which they pertain. Where a range of values is provided, it will be understood that the upper and lower limit, and each intervening value between the upper and lower limit of the range, is encompassed within the embodiments. Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements. Accordingly, in some embodiments, the numerical parameters set forth in the description and claims are approximations that can vary depending upon the desired properties sought to be obtained by a particular embodiment. The term “substantially,” when applied to modify a parameter or characteristic herein, will be read in the context of the invention and in light of the knowledge in the art to provide certainty, e.g., by using a standard that is recognized in the art for measuring the meaning of substantially as a term of degree, or by ascertaining the scope as would one of skill. Generally, the nomenclature used and procedures performed herein are those known in field(s) relating to that of one or more aspects of the invention, and are those that will be well-known and commonly employed in such field(s). Standard techniques and procedures will be those generally performed according to conventional methods in the art. Unless defined otherwise, all technical and scientific terms herein have the meaning as commonly understood by a person having ordinary skill in the art to which this invention belongs, who as a shorthand may be referred to simply as “one of skill.”

### B. AMANITA MUSHROOMS AND EXTRACTS THEREOF

A. muscaria, or the fly agaric, is a mycorrhizal basidiomycete fungus native to temperate and boreal regions of the Northern Hemisphere. Certain Amanita species, with A. muscaria prime among them, are known for their psychoactive properties and neurotropic effects.

A. muscaria will be understood to include the A. muscaria var. Muscaria (Euro-Asian fly agaric), A. muscaria var. flavivolvata (American fly agaric), A. muscaria var. guessowii (American fly agaric, yellow variant), and A. muscaria var. inzegae (“Inzenga's” fly agaric). Also within the scope and spirit of the invention, and which therefore shall be considered within the definition of A. muscaria or as equivalents thereof, are such other ibotenic acid and muscimol containing Amanitas, as will be appreciated by those of skill.

The psychoactive properties of A. muscaria are generally attributed to the centrally-active alkaloids ibotenic acid and muscimol. While few consume raw A. muscaria, consumption of parboiled A. muscaria has a history of practice, as parboiling A. muscaria mushrooms can detoxify them to yield an edible product (Rubel and Arora, Economic Botany, 2008; 62, 157-173). However, such compositions may still comprise levels of toxic compounds, such as ibotenic acid, that limit their therapeutic use.

a. Amanita Mushroom Extract Content

In some aspects, provided herein are extracts of A. muscaria. Herein, extract refers to a botanical extract, e.g., an A. muscaria mushroom extract from a fungal source. In some embodiments, the provided A. muscaria extracts are not subjected to isolation, purification, or other processing to obtain specific active compounds, separate from other constituents.

In some embodiments, a provided A. muscaria extract may be a “purified extract,” which herein refers to a botanical extract that has undergone further processing after preparation, such as soaking or heating the preparation in water and/or alcohol, agitating, cooling the resulting liquid, straining, filtering, and removing unwanted products (repeating if necessary), and then evaporating sufficient liquid solvent to obtain a desired concentration (or entirely, to obtain a solid precipitate), or using a spray dryer to create a purified dried powder. In some embodiments, the A. muscaria extract is obtained as a liquid distillate. In some embodiments, the A. muscaria extract is obtained as a ground powder.

In some embodiments, a provided A. muscaria extract comprises muscimol. Muscimol (C₄HN₂O₂) is one of the main psychoactive components of A. muscaria. Muscimol is known to be an agonist for GABA_{A} receptors (Johnston, Eurochem Res. 2014; 39(10):1942-7). When binding to a GABA_{A} receptor, muscimol activates the receptor, causing anxiolytic, anticonvulsant, amnesic, sedative, hypnotic, euphoriant, and muscle relaxant properties. Muscimol may also cause hallucinogenic effects in a subject.

While the toxicokinetics of muscimol remain largely unknown, a fast absorption of muscimol is presumed based on the rapid onset of symptoms after ingestion of Amanita mushrooms (Stribrny et al., Int. J. Legal Med., 2012; 126, 519-524). Moreover, muscimol is readily excreted into the urine, where it can be detected within an hour after ingestion (Stribrny et al., Int. J. Legal Med., 2012; 126, 519-524). Preclinical studies in rats investigated the distribution and metabolism of muscimol. Baraldi and colleagues (Neuropharmacol., 1979; 18, 57-62) investigated these in the brain and other tissues in the rat. Male Sprague Dawley rats (n=3-5) received either an IV injection of muscimol (3Hmethylene-muscimol, 8 μmol/kg or 12.1 Ci/mmol) or intracerebroventricular injection (1200 nmol/kg), and metabolites from blood and brain were extracted. Intravenous administration of muscimol resulted in an uneven distribution at various brain regions, with the highest muscimol concentration being in the substantia nigra, the colliculi and the hypothalamus. However, only a small amount of the dose injected (approximately 0.02% in a 150 g rat) crossed to the brain (approximately 200 pmol/g tissue). On the other hand, intracerebroventricular injection of muscimol led to a distribution of muscimol that was approximately 2 times higher in the corneum stratum compared to the hypothalamus, hippocampus and cortex. After intravenous injection, muscimol was removed very rapidly from the blood, and blood concentration was below limits of detection within 180 min. Upon disappearance from the blood, there was a paralleled increase in plasma levels of radioactive muscimol metabolites. Furthermore, there were no reports of adverse effects or toxicity in this study, suggesting that an intravenous injection of muscimol (8 μmol/kg) was well-tolerated.

In some embodiments, the A. muscaria extract comprises 1% to 5% of muscimol. In embodiments, the A. muscaria extract comprises about 2%, 3%, 4%, or 5% of muscimol. In embodiments, the A. muscaria extract comprises 2.5%, 2.6%, 2.7%, 2.8%, 2.9%, 3.0%, 3.1%, 3.2%, 3.3%, 3.4%, or 3.5% muscimol. In embodiments, the concentration of muscimol is the % w/w. In embodiments, the concentration of muscimol is the % w/v. In embodiments, the A. muscaria extract comprises less than 10,000 μg/g, 15,000 μg/g, 20,000 μg/g, 25,000 μg/g, 30,000 μg/g, 35,000 μg/g, 40,000 μg/g, 45,000 μg/g or 50,000 μg/g of muscimol.

In some embodiments, a provided A. muscaria extract comprises muscarine. Muscarine (C₉₀H₂₀NO₂+) is a further component of A. muscaria that is typically found in smaller quantities than ibotenic acid and muscimol. Muscarine is a nonselective agonist of the muscarinic acetylcholine (ACh) receptors (Broadley et al., Molecules, 2001; 6(3):142-193), which may be toxic in concentrations found in certain species, but not typically in those found in A. muscaria.

Muscarine induces short-lived symptoms, indicative of activation of muscarinic receptors, including vomiting, hypotension, nausea, abdominal cramping, diarrhea, lacrimation, hypersalivation, bronchoconstriction, chills, tremor, bronchial secretions, sweating, gastric acid secretion, miosis, blurred vision, polyuria, diaphoresis, rhinorrhea, headache, anxiety and bradycardia (Lurie et al., Clin. Toxicol. (Phila), 2009; 562-565). Despite such symptoms, it is important to consider that in terms of muscarinic syndrome from ingestion of A. muscaria, one would have to ingest enormous quantities of A. muscaria before a muscarinic effect is elicited.

Muscarine mimics the action of acetylcholine (ACh) on muscarinic-type ACh receptors. In general, quaternary ammonium compounds such as muscarine are poorly absorbed after oral exposure. Once absorbed, muscarine is quickly distributed throughout the body, and clinical signs can develop in humans within 30 min to 2 hours. Muscarine is not metabolized by acetylcholinesterase like the neurotransmitter ACh. Instead, muscarine leaves the blood via renal clearance and exits the body in urine (Bartholow, A practical treatise on materia medica and therapeutics, 1908; 6). While detailed pharmacokinetic studies on muscarine are scarce, recently, Sai Latha et al. (Sci. Rep., 2020; 10, 13669) investigated the pharmacokinetics and biodistribution of a synthetic radiolabelled muscarine. In this study, radiolabelled muscarine chloride (1.5 mCi 99mTcO4−) was injected through the dorsal ear vein of normal, healthy female New Zealand rabbits (n=3). Blood was withdrawn at periodic intervals and radioactivity measured using gamma scintigraphy. Approximately 42% of the radiolabelled muscarine reached the blood within 5 min after administration. A significant amount of the toxin was rapidly concentrated in the thorax and head, demonstrating early muscarinic responses such as miosis and salivation. Muscarine was also detected in the spleen following a decline of blood level by 87.89% after 1 hr of ingestion. Muscarine was detected in the bladder, suggesting renal clearance. After 24 hours, a large proportion of muscarine was accumulated in the liver, which provides an explanation for the hepatoxicity that was observed in the study in mice.

In some embodiments, the A. muscaria extract comprises 0.02% to 0.08% of muscarine. In some embodiments, the A. muscaria extract comprises about 0.02%, 0.03%, 0.04%, 0.05%, 0.06%, 0.07%, or 0.08% of muscarine. In some embodiments, the concentration of muscimol is the % w/w. In some embodiments, the concentration of muscimol is the % w/v. In some embodiments, the A. muscaria extract comprises less than 250 μg/g, 300 μg/g, 350 μg/g, 400 μg/g, 450 μg/g, 500 μg/g, 550 μg/g, 600 μg/g, or 650 μg/g of muscarine.

In some embodiments, a provided A. muscaria extract comprises ibotenic acid. Ibotenic acid (C₅HN₂O₄) is a conformationally-restricted analogue of the neurotransmitter glutamate that acts as a non-selective glutamate receptor agonist. Ibotenic acid also acts as a neurotoxin and has been employed as a “brain-lesioning agent” through cranial injections in scientific research. In typical samples, A. muscaria contains more ibotenic acid than muscimol. At least some of the ibotenic acid in A. muscaria is converted by decarboxylation to muscimol in the acid environment of the stomach. Ibotenic acid therefore can serve as a prodrug to muscimol. However, if too much ibotenic acid is ingested, it can cause stomach irritation, nausea, diarrhea, sweating and salivation, lethargy and drowsiness, ataxia, and other somatic symptoms, as well as psychological symptoms such as confusion, euphoria, visual and auditory hallucinations, sensations of floating, distortions of space and time, and retrograde amnesia (Moss et al. Clin Toxicol (Phila). 2019; 57(2):99-103).

Ibotenic acid is the precursor of muscimol. It is metabolized by decarboxylation in the stomach, liver, and brain to equal amounts of muscimol (Nielsen et al., J. Neurochem., 1985; 45, 725-731). While the toxicokinetics of ibotenic acid remain largely unknown, a fast absorption of ibotenic acid is presumed after ingestion of Amanita mushrooms due to the rapid appearance of related symptoms (Stnbrny et al., Int. J. Legal Med., 2012; 126, 519-524). Similar to muscimol, ibotenic acid is also readily excreted into the urine, where it can be detected within an hour after ingestion (Stnbrny et al., Int. J. Legal Med., 2012; 126, 519-524).

In some embodiments, the A. muscaria extract comprises about 0.0005% to 0.005% of ibotenic acid. In embodiments, the A. muscaria extract comprises about 0.0005%, 0.0010%, 0.0015%, 0.002%, 0.0025%, 0.003%, 0.0035%, 0.004%, 0.0045%, or 0.005% of ibotenic acid. In some embodiments, the concentration of ibotenic acid is the % w/w. In some embodiments, the concentration of ibotenic acid is the % w/v. In some embodiments, the A. muscaria extract comprises less than 40 μg/g, less than 30 μg/g, less than 20 μg/g, less than 10 μg/g, or less than 5 μg/g of ibotenic acid. In some embodiments, the A. muscaria extract comprises 40 μg/g or less, 30 μg/g or less, 20 μg/g or less, 10 μg/g or less, or 5 μg/g or less of ibotenic acid.

In some embodiments, the A. muscaria extract is substantially or essentially devoid of stizolobinic acid. In some embodiments, the A. muscaria extract comprises less than 40 μg/g of stizolobinic acid. In some embodiments, the A. muscaria extract is substantially or essentially devoid of heavy metals. In some embodiments, the A. muscaria extract is substantially or essentially devoid of one or more of cadmium, arsenic, lead, and mercury. In some embodiments, the A. muscaria extract comprises less than 0.09 ppm of cadmium, 0.03 ppm of arsenic, 0.09 ppm of lead and 0.02 ppm of mercury. In some embodiments, the A. muscaria extract comprises no more than 40 μg/g stizolobinic acid and no more than 0.09 ppm of cadmium, 0.03 ppm of arsenic, 0.09 ppm of lead and 0.02 ppm of mercury.

In some embodiments, the A. muscaria extract comprises less than or no more than the limits for respective pesticides in USP General Chapter 561, “Articles of Botanical Origin” (USP 561). In some embodiments, the extract is also in compliance with EPA (40 C.F.R. § 180) and FDA action levels (21 C.F.R. §§ 109, 509). In some embodiments, the extract is manufactured in compliance with GLP or GMP requirements.

One of skill may use known methods to determine the presence of contaminants, one such method being HPLC-MS/MS. In some embodiments, HPLC-MS/MS may be used to determine the presence and concentration of other target compounds in the A. muscaria extract. In some embodiments, HPLC-MS/MS is used to determine the concentration of muscimol, ibotenic acid, and/or muscarine.

In some embodiments, the A. muscaria extract comprises muscimol and muscarine in a ratio of at least 1:1. In some embodiments, the muscimol to muscarine ratio is from about 1:1 to about 100:1. In embodiments, the muscimol to muscarine ratio is about 1:1, 5:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, and 100:1.

In some embodiments, the A. muscaria extract comprises muscimol and ibotenic acid in a ratio of at least 3:1. In some embodiments, the muscimol to ibotenic acid ratio is from about 3:1 to about 150:1. In some embodiments, the muscimol to ibotenic acid ratio is about 10:1, 15:1, 20:1, 25:1, 30:1, 35:1, 40:1, 45:1, 50:1, 55:1, 60:1, 65:1, 70:1, 75:1, 80:1, 85:1, 90:1, 95:1, 100:1, 105:1, 110:1, 115:1, 120:1, 125:1, 130:1, 135:1, 140:1, 145:1, 150:1, 155:1, 160:1, 165:1, 170:1, 175:1, 180:1, 185:1, 190:1, 195:1, or 200:1.

In some embodiments, the A. muscaria extract comprises muscimol and ibotenic acid in a ratio of about 1000:1 to 3000:1, 1500:1 to 2500:1, or 1750:1 to 2250:1. In some embodiments, the A. muscaria extract comprises muscimol and ibotenic acid in a ratio of at least 1000:1, at least 1100:1, at least 1200:1, at least 1300:1, at least 1400:1, at least 1500:1, at least 1600:1, at least 1700:1, at least 1800:1, at least 1900:1, at least 2000:1, at least 2100:1, at least 2200:1, at least 2300:1, at least 2400:1, at least 2500:1, at least 2600:1, at least 2700:1, at least 2800:1, at least 2900:1, or at least 3000:1. In some embodiments, the A. muscaria extract comprises muscimol and ibotenic acid in a ratio of about 1500:1, about 1510:1, about 1520:1, about 1530:1, about 1540:1, about 1550:1, about 1560:1, about 1570:1, about 1580:1, about 1590:1, about 1600:1, about 1610:1, about 1620:1, about 1630:1, about 1640:1, about 1650:1, about 1660:1, about 1670:1, about 1680:1, about 1690:1, about 1700:1, about 1710:1, about 1720:1, about 1730:1, about 1740:1, about 1750:1, about 1760:1, about 1770:1, about 1780:1, about 1790:1, 1800:1, about 1810:1, about 1820:1, about 1830:1, about 1840:1, about 1850:1, about 1860:1, about 1870:1, about 1880:1, about 1890:1, about 1900:1, about 1910:1, about 1920:1, about 1930:1, about 1940:1, about 1950:1, about 1960:1, about 1970:1, about 1980:1, about 1990:1, or about 2000:1, about 2010:1, about 2020:1, about 2030:1, about 2040:1, about 2050:1, about 2060:1, about 2070:1, about 2080:1, about 2090:1, or about 3000:1. While the toxicity of ibotenic acid is recognized, see, e.g., US20140004084 and WO2022132691, no known reference at the time of filing contemplates or achieves muscimol to ibotenic acid ratios in excess of 1000:1, much less ratios in excess of 1800:1, as is accomplished by the present inventor in providing the disclosed A. muscaria extracts.

In some embodiments, the A. muscaria extract comprises between about 0.5% and about 5.0% w/w or w/v muscimol, between about 0.05% and about 0.5% w/w or w/v muscarine, and less than about 0.05% w/w or w/v ibotenic acid.

In some embodiments, the A. muscaria extract comprises between about 0.001% w/w or w/v muscazone, and about 0.01% w/w or w/v muscazone.

In some embodiments, the A. muscaria extract comprises less than 18 mg/g of muscimol, less than 600 μg/g of muscarine, and less than 20 μg/g of ibotenic acid.

In some embodiments, the A. muscaria extract comprises at least 15,000 μg/g. In some embodiments, as illustrated in Table 1 below, the A. muscaria extract comprises between about 500 μg/g and 20,000 μg/g, wherein the range is inclusive.

In some embodiments, the A. muscaria extract comprises less than 250 μg/g of ibotenic acid. In some embodiments, as illustrated in Table 1 below, the A. muscaria extract comprises between about 240 μg/g and about 50 μg/g.

In some embodiments, the A. muscaria extract comprises 5% or less of any one of muscimol, muscarine, and ibotenic acid. In some embodiments, the A. muscaria extract comprises 4% or less of any one of muscimol, muscarine, and ibotenic acid. In some embodiments, the A. muscaria extract comprises 3% or less of any one of muscimol, muscarine, and ibotenic acid. In some embodiments, the A. muscaria extract comprises about 2.5% to 3.5% muscimol. In some embodiments, the A. muscaria extract comprises 2.5%, 2.6%, 2.7%, 2.8%, 2.9%, 3.0%, 3.1%, 3.2%, 3.3%, 3.4%, or 3.5% muscimol. In some embodiments, the A. muscaria extract comprises 2.8% muscimol. In some embodiments, the described concentrations are mass percent concentrations, such as w/w %, e.g., μg/g.

In some embodiments, the A. muscaria extract comprises 0.1% or less of muscarine and/or ibotenic acid. In some embodiments, the A. muscaria extract comprises 0.09% or less of muscarine and/or ibotenic acid. In some embodiments, the A. muscaria extract comprises 0.08% or less of muscarine and/or ibotenic acid. In some embodiments, the A. muscaria extract comprises 0.07% or less of muscarine and/or ibotenic acid. In some embodiments, the A. muscaria extract comprises 0.06% or less of muscarine and/or ibotenic acid. In some embodiments, the A. muscaria extract comprises 0.05% or less of muscarine and/or ibotenic acid. In some embodiments, the A. muscaria extract comprises about 0.02% to 0.08% of muscarine. In some embodiments, the A. muscaria extract comprises 0.02%, 0.03%, 0.04%, 0.05%, 0.06%, 0.07%, or 0.08% of muscarine. In some embodiments, the A. muscaria extract comprises about 0.05% muscarine. In some embodiments, the described concentrations are mass percent concentrations, such as w/w %, e.g., μg/g.

In some embodiments, the A. muscaria extract comprises 0.005% or less of ibotenic acid. In some embodiments, the A. muscaria extract comprises 0.004% or less of ibotenic acid. In some embodiments, the A. muscaria extract comprises 0.003% or less of ibotenic acid. In some embodiments, the A. muscaria extract comprises 0.002% or less of ibotenic acid. In some embodiments, the A. muscaria extract comprises 0.001% to 0.002% of ibotenic acid. In some embodiments, the A. muscaria extract comprises 0.001%, 0.0011%, 0.0012%, 0.0013%, 0.0014%, 0.0015%, 0.0016%, 0.0017%, 0.0018%, 0.0019%, or 0.002% of ibotenic acid. In some embodiments, the A. muscaria extract comprises about 0.0015% ibotenic acid. In some embodiments, the described concentrations are mass % concentrations, such as w/w %, e.g., μg/g.

In some embodiments, the A. muscaria extract comprises about 2.8% muscimol, about 0.05% muscarine, and about 0.0015% ibotenic acid. In some embodiments, the A. muscaria extract is substantially free from ibotenic acid, stizolobinic acid and heavy metals. In some embodiments, the A. muscaria extract comprises less than 20 μg/g ibotenic acid, less than 40 μg/g stizolobinic acid, less than 0.09 ppm of cadmium, less than 0.03 ppm of arsenic, less than 0.09 ppm of lead, and less than 0.02 ppm of mercury. In some embodiments, the properties of a provided A. muscaria extract meet the testing specifications shown in Table 1. Table 2 and Table 3 show an additional A. muscaria extract having content that meets the specifications of exemplary A. muscaria extract AME-1.

TABLE 1
Testing Specifications for Exemplary A. muscaria Extract AME-1

| Parameter | Specification | | Method |
|---|---|---|---|
| Color | Brown | | Organoleptic |
| Taste | Similar to meat gravy | | Organoleptic |
| Smell | Forest mushroom scent | | Organoleptic |
| Muscimol | <30000 | μg/g | HPLC-MS/MS |
| Muscarine | <550 | μg/g | HPLC-MS/MS |
| Ibotenic acid | <20 | μg/g | HPLC-MS/MS |
| Stizolobinic acid | <40 | μg/g | HPLC-MS/MS |
| Arsenic | <0.03 | ppm | HPLC-MS/MS |
| Cadmium | <0.09 | ppm | HPLC-MS/MS |
| Mercury | <0.02 | ppm | HPLC-MS/MS |
| Lead | <0.09 | ppm | HPLC-MS/MS |

| | | |
|---|---|---|
| Pesticides | Conforms | Conforms |

b. Potency and Purity

In some aspects, provided herein are A. muscaria compositions of specific potency and purity. In some embodiments, potency is equivalent to concentration. In some embodiments, potency can be determined by w/w %, e.g., by dividing the weight of a compound to be assessed with the total weight of an extract. Potency and purity may be determined according to methods known to one of skill in the art.

In some embodiments, the potency of muscimol in a disclosed A. muscaria composition, such as an extract, is at least 0.25%, 0.5%, 0.75%, 1%, 1.25%, 1.5%, 1.6%, 1.65%, 1.75%, 2.0%, 2.5%, 2.75%, or 3%. In some embodiments, the potency of muscimol in a disclosed A. muscaria composition is about 0.25% to 5%, 0.5% to 4%, 0.75% to 3%, 1% to 2%, 1.25% to 1.75%, 1.5% to 1.7%. In some embodiments, a disclosed A. muscaria compositions, such as an extract, has a muscimol potency of about 1.5%, 1.6%, 1.7%, 1.8%, 1.9%, 2.0%, 2.1%, 2.2%, 2.3%, 2.4%, 2.5%, 2.6%, 2.7%, 2.8%, 2.9%, or 3.0%.

In some embodiments, the potency of muscarine in a disclosed A. muscaria composition, such as an extract, is less than 0.02%, 0.03%, 0.04%, 0.05%, 0.06%, 0.07%, 0.08%, 0.09% or 1%. In some embodiments, the potency of muscarine in a disclosed A. muscaria composition is about 0.01% to 0.3%, 0.025% to 0.2%, 0.03% to 0.175%, or 0.04% to 0.15%. In embodiments, a disclosed A. muscaria compositions, such as an extract, has a muscarine potency of about 0.03%, 0.04%, 0.05%, 0.06%, 0.07%, 0.08%, 0.09%, 0.1%, 0.15%, 0.2%, 0.25%, 0.3%, 0.35%, or 0.4%

In some embodiments, the A. muscaria extract comprises ibotenic acid having a potency of less than 0.025%, 0.02%, 0.015%, 0.01%, 0.009%, 0.008%, 0.007%, 0.006%, 0.005%, 0.004%, 0.0035%, 0.003%, 0.0025%, 0.002%, 0.0015%, or 0.001%.

In some embodiments, the A. muscaria extract has a muscimol purity of at least 90%, such as about 90%, 91%, 92%, 93%, 94%, 95%, 96%, 97%, 98%, 99%, and greater than 99%. In embodiments, the purity of a compound is determined by dividing the weight of the compound by the total weight of the compounds against which its purity is measured (i.e., as a w/w %).

In some embodiments, the A. muscaria extract, which may optionally be further concentrated, is standardized. In some embodiments, the A. muscaria extract is standardized to a muscimol purity of 90% or greater. A “standardized” extract refers to an extract comprising a specified quantity of a standardized ingredient, which may be a bioactive compound such as muscimol. Thus, In some embodiments, an amount of the bioactive compound, such as an amount of muscimol, is standardized to a particular concentration (e.g., w/w or w/v % of the extract). In some embodiments, an A. muscaria extract will be standardized so as to contain by weight percent an amount of muscimol (i.e., mg muscimol per mg or mL of extract, depending on whether such extract is a dry powder or a liquid) of between 0.5% and 5.0% w/w or w/v muscimol, wherein the range is inclusive. In some embodiments, the A. muscaria extract will contain by weight percent an amount of muscarine (i.e., mg muscarine per mg extract) of at least 0.05% w/w or w/v muscarine. Other standardizations of w/w or w/v muscimol will include, for example, amounts of between 5.0% and 10.0% or greater than 10% muscimol and also including amounts lower than those explicitly listed above.

Standardization may be accomplished by methods such as measuring a concentration of compound in an extract to be standardized, determining a desired concentration of the compound when standardized, determining an amount of excipient necessary to obtain the desired (standardized) concentration, and then adding the amount of excipient necessary to obtain the desired (standardized) concentration, resulting in a standardized extract. An excipient may be a dry or liquid excipient, to create a dry powder or liquid standardized extract.

The concentration of the standardized compound in the standardized extract may be measured after adding one or more portions of excipient or after the standardized extract is prepared, to confirm the standardization method and for quality control.

In some embodiments, the A. muscaria extract is further concentrated so the bioactive compounds (including, and in particular muscimol) are increased in total concentration from an initial extract, such as an increase in w/w % (for a powder extract) or w/v % (for a liquid extract), in an amount such as by at least 5%, at least 10%, at least 15%, at least 20%, at least 25%, at least 30%, at least 40%, at least 45%, at least 50%, at least 60%, at least 70%, at least 80%, at least 90%, at least 100% (2×), at least 125%, at least 150%, at least 175%, at least 200% (3×), at least 250%, at least 300% (4×), at least 400% (5×), at least 500% (6×), at least 600% (7×), at least 700% (8×), at least 800% (9×), at least 900% (10×), and in amounts of 1,000% or more.

In some embodiments having a further concentrated extract, the bioactive compounds in the A. muscaria extract may be increased by a like amount. For example, where a pharmaceutical composition includes a 10× concentrated A. muscaria extract, it will contain bioactive A. muscaria compounds including muscimol, muscarine, and ibotenic acid, and will as an example contain by weight/weight or weight/volume percent an amount of muscimol of 5% or less, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, or at least 30% w/w muscimol.

Exemplary methods of concentrating an extract include, e.g., evaporating a portion or an entirety of a solvent to create a volume of concentrated slurry at a desired concentration.

### C. METHODS FOR PREPARING A. MUSCARIA EXTRACTS

In some aspects, provided herein are methods of making A. muscaria extracts. Exemplary methods of making such extracts are described in Applicant's International Appl. No. PCT/CA2022/050378, which is incorporated by reference as if fully set forth herein.

In an exemplary extraction and concentration procedure, A. muscaria mushrooms (below as shorthand and for convenience, “mushrooms”) are harvested or gathered, or are otherwise obtained as fresh mushrooms. Alternatively, mushrooms may be obtained dried. Optionally, the mushrooms may be frozen for a period of storage prior to further processing, upon which time the mushrooms may be thawed.

Mushroom caps have the highest concentration of target compounds, and thus in certain preferred embodiments, the caps are removed from the stipes prior to processing, and only the caps are used, therefore leading to greater overall yields of muscimol, and higher potency. However, both caps and stipes may be used; as can stipes alone.

In some embodiments, the mushrooms utilized in the process of the invention are sufficiently dry, having a moisture content of from about 2% to about 3% moisture by weight. If not sufficiently dry when harvested, the mushrooms may be dried (that is, dehydrated) in a dehydrator or by application of heat in any conceivable method known to those of skill.

It will be appreciated that if dried below 2%, or at too high of a temperature, muscimol and ibotenic acid could be degraded, reducing the final yield of muscimol that obtained by the process. The appropriate amount of drying may be determined by practice of ordinary skill for the embodiment of the invention being carried out. Drying the harvested mushrooms is also an optional step, and should not be construed as limiting. In some embodiments where drying is performed, drying may be completed at a maximum temperature of 50° C.

In some embodiments, the mushrooms are ground to a powder by a food processor, coffee grinder, blender, or similar device. Mushrooms also may be chopped, pulverized, and/or otherwise rendered to smaller pieces or particles by methods and devices known to those of skill. Although steps below may be described as using “ground mushrooms,” it therefore will be appreciated that such mushrooms may also be whole or in pieces or in particles of various sizes, all of which are within the scope of the methods, but will have properties that may differ according to ordinary skill, and that smaller pieces or particles may require lower processing times and/or provide an extract of greater potency.

In some embodiments, a small batch of mushrooms or the ground powder thereof is optionally analyzed to determine whether the muscimol, muscarine, and ibotenic acid are within safety and production specifications. In addition, the batch may or may not be analyzed for heavy metal and pesticide content. The analysis may be performed by any means known to those of skill capable of completing such an analysis, e.g., high performance liquid chromatography tandem mass spectrometry (HPLC-MS/MS). In some embodiments, the batch must include at least about 500 micrograms per gram of muscimol to proceed with the method of the invention.

Water is then obtained, the pH is determined, and is maintained at about 7. The water is then boiled, and the mushrooms are placed in the boiled water and stirred. In embodiments, the ratio of ground mushroom caps to water may be about 1 gram of mushroom per 40 mL of water.

The temperature of the mixture of water and ground mushrooms is maintained at 95° C. to 100° C. The mixture is then stirred for between about 5 minutes to about 180 minutes at between 700 rpm and 2500 rpm. It will be readily appreciated that the duration of stirring only be that which is sufficient to mix the solution.

In some embodiments, the mixture is then filtered to remove solids. In some embodiments, pressure is applied during the filtration process. In some embodiments, the filtrate may be collected in a flask. In some embodiments, the pH of the filtrate may be adjusted to between about 5-6, or lower, by adding an acid to the flask. In some embodiments, the acidic mixture is then refluxed through a distillation apparatus to concentrate the extract. Other mechanisms of concentrating known to one of skill may be applied to methods described herein.

In some embodiments, the acidified mushroom filtrate or extract is concentrated to accelerate decarboxylation of ibotenic acid into muscimol. In some embodiments, the concentrating step comprises heating the acidified mushroom extract. In some embodiments, the duration of heat exposure is from about 0.5 hours to about 6 hours. In some embodiments, the extract is heated from about 75° C. to about 177° C. (200° F.-350° F.). In some embodiments, the concentration step comprises applied pressure. In some embodiments, the applied pressure is between about 10 psi and about 25 psi.

In some embodiments, a condenser may be used, a non-limiting example of which is a Pyrex Graham condenser, which may be attached to another flask, such as a round bottom flask, for collection. The Graham condenser may be placed in a downward position for distillation, so that the acidic mixture enters the condenser at the bottom. The unused neck is stoppered with a plug. In some embodiments, steps are taken to avoid light exposure throughout the process, due to the potential for muscimol degradation.

The condenser coil is cooled with chilled water pumped in by a pump. The setup is heated and under pressure, such as with a heating mantle. In some embodiments, the distillate is additionally stirred to facilitate even heating. Regardless, high heat is applied (e.g., placing the heating mantle on “high”) until distillation begins and then is adjusted so that the rate of distillate collection is about 1 drop per second, for example. In some embodiments, such “high” heat is between about 110° C. to about 130° C. Depending on the length of distillation, the muscimol content may be increased after distillation by, for example, at least 25%, at least 30%, at least 40%, at least 50%, at least 60%, at least 75%, at least 100%, at least 150%, at least 200%, or at least 250%, compared to the content before such distillation.

### Example 1: Preparation of an A. muscaria Extract

A. muscaria mushrooms were obtained from Vashon Island, WA. The mushrooms were harvested in autumn, dried to a moisture content of from about 2% to about 4% by weight, sealed in plastic 4 L bags, and stored frozen in darkness at −15° C. until used. The mushrooms were allowed to thaw to room temperature, were then removed from the plastic bags, and were then inspected to ensure dryness so they can be easily ground to a powder. Dryness was determined by attempting to snap mushroom caps in half. Mushroom caps in the desired range of dryness snapped in half. This was determined one hour prior to beginning the extraction process.

Mushrooms that were insufficiently dry were dehydrated in a forced air food dehydrator (internal dimensions, 30 cm×30 cm×30 cm) at 50° C. for 90 minutes. Drying continued until each mushroom was sufficiently dehydrated (about 2-3% moisture content by weight). The dried mushrooms were mixed with distilled water. The pH of the distilled water was first measured after calibration with a pH meter. The pH of the distilled water was about 7. Since distilled water was stored in a plastic container, the distilled water was boiled to remove any organic material that may have leached into the water from the plastic container. 1000 mL of the distilled water was placed in a 2000 mL Pyrex beaker and boiled at 100° C. for 10 minutes in a microwave oven.

The mushroom caps were ground by a coffee grinder. Caps were ground for 45 seconds.

The beaker of boiled water was removed from the microwave oven and placed on a heating plate. Twenty-five grams of the ground mushroom caps were added to the hot water and gently stirred with a glass rod for 10 minutes. The temperature was maintained at 95° C. The ratio of mushroom caps to distilled water was 25 grams to 1000 mL water. The mixture of the ground mushroom caps in the heated water in the beaker was then filtered. A Buchner funnel was set up for vacuum filtration. A 23 gram piece of 10 cm×10 cm×5 cm glass wool was cut in a circular shape approximately the same diameter as the Buchner funnel. It was placed on top of the glass filter in the funnel. Four grams of cheese cloth measuring 25 cm×30 cm was folded into four layers and placed on top of the glass wool. The cheese cloth captured larger particles while the glass wool captured smaller particles.

The filter setup was attached to a 1000 ml Erlenmeyer flask. The filter layers were seated by pouring 100 ml of the distilled water that was earlier boiled, through the setup with vacuum turned on. Filtrate was removed from the flask after pouring water through the filters.

To filter the mixture of ground mushroom and water, the mixture was allowed to settle for two minutes in the beaker. The mixture was then slowly decanted into the filter without the ground mushroom powder at the bottom of the beaker to pour into the filter until near the end of the procedure. This reduced clogging and maintained a better flow through the filter. The vacuum pump was turned on to begin to filter the extract, at a pressure of −0.7 bar.

When the extract was filtered, the filter set up as vacuum flushed with 30 ml of the distilled water that was previously boiled. The process yields from about 925 mL to about 975 mL of filtrate in the flask, when performed as a single run. The filtrate appeared cloudy.

The pH of the filtrate was adjusted from the pH of about 7 (the pH of the distilled water) to a pH of about 3.0 by the addition of 1M hydrochloric acid. After addition of the hydrochloric acid, the filtrate was refluxed through a Pyrex Graham condenser having a coolant-jacketed spiral coil running the length of the condenser serving as the vapor-condensate path. The length of the Graham condenser was 43 cm. The length of the water jacket was 30 cm and it had 24/60 glass joints. The Graham condenser was attached to a 2000 ml round bottom flask for collection. The Graham condenser was in a downward position for distillation. The unused neck was stoppered with a glass 24/40 plug.

A round bottom flask was secured to the condenser with burette/test tube clamps that were attached to a generic 60 cm high ring stand. Aluminum foil was wrapped around the round bottom flask and up the condenser part way to lower light intensity inside the round bottom flask during refluxing. The Erlenmeyer flask was attached to the inlet of the Graham condenser.

The condenser coil was cooled with cold water pumped in by a pump. The setup Graham condenser was heated with a heating mantle/magnetic stirrer. The distillation was performed in a fume hood. Distillation took place at 250° F. (˜121° C.) at 15 psi (˜1.0 bar). A 2.5 cm magnetic stir bar was placed in the pH distillate to be distilled before starting distillation. During distillation, the magnetic stirring was provided at from about 30 rpm to about 60 rpm, to prevent bumping. The heating level was set on high until the distillation began and was then adjusted so that 1 drop per second was observed and collected.

The refluxed extract was then distilled for a period of 3 hours. Samples were taken prior to distillation, after 1 hour, 2 hours, and at 3 hours of distillation, as discussed further below. After distillation, the filtrate was cooled to 3° C. in a refrigerator. After about 3 hours of cooling, there was coagulated material suspended in the filtrate. The coagulated material in the filtrate was removed via vacuum filtration through the vacuum filtration setup described above (without the cheesecloth). After filtration, the filter setup was rinsed under vacuum with 30 mL of distilled water into the filtrate.

HPLC-MS/MS was used to analyze mushrooms and extracts at various stages of the process. The starting material for the extraction process had the following concentrations: muscimol (625 μg/g), muscarine (340 μg/g), and ibotenic acid (2930 μg/g).

HPLC-MS/MS analysis was completed at hourly timepoints of the 3 hour distillation. Prior to distillation, the extract contained 44.3 μg/g muscimol, 12.4 μg/g muscarine, and 228 μg/g ibotenic acid. One hour after distillation, the amount of muscimol increased from 44.3 μg/g to 144 μg/g, while the amount of ibotenic acid decreased from 228 μg/g to 44.9 μg/g. After two hours of distillation, muscimol levels increased to 162 μg/g, ibotenic acid decreased to 7.37 μg/g, and muscarine levels were 12.7 μg/g. After 3 hours of distillation, levels of muscimol decreased from 162 μg/g to 146 μg/g, levels of muscarine were measured at 12.1 μg/g, and ibotenic acid levels decreased to 1.38 μg/g.

Quantitative analyses of the cadmium, arsenic, mercury, and lead content of the mushrooms were also performed by inductively coupled plasma mass spectrometry (“ICPMS”). The cadmium content of the heated, ground mushroom powder was 10.2 parts per million (“PPM”), the arsenic content was 1.67 PPM, the mercury content was 1.18 PPM, and the lead content was 0.865 PPM. Prior to the start of reflux, the cadmium content was 0.073 PPM, the arsenic content was 0.018 PPM, the mercury content was below detection limits (less than 0.001 PPM), and the lead content was 0.011 PPM. The extract obtained via this process is illustrated in Tables 2 and 3 below, showing Certificates of Analysis (COAs).

TABLE 2
COA of A. muscaria extract meeting specifications
of exemplary extract AME-1
Extract Amount: 20 g

| Description Assay | Method | Result | | Pass or Fail |
|---|---|---|---|---|
| Muscimol | Non-compendial | 146 | μg/g | Pass |
| | in-house method by | | | |
| | HPLC-MS/MS | | | |
| Muscarine | Non-compendial | 12.1 | μg/g | Pass |
| | in-house method by | | | |
| | HPLC-MS/MS | | | |
| Ibotenic acid | Non-compendial | 1.38 | μg/g | Pass |
| | in-house method by | | | |
| | HPLC-MS/MS | | | |
| Cadmium | EPA 3052 | 0.073 | μg/g | Pass |
| Arsenic | EPA 3052 | 0.018 | μg/g | Pass |
| Mercury | EPA 3052 | ND μg/g | | Pass |
| Lead | EPA 3052 | 0.011 | μg/g | Pass |

TABLE 3
COA of A. muscaria extract meeting specifications
of exemplary extract AME-1
Extract Amount: 4300 g (solid)

| Description Assay | Method | Result | | Pass or Fail |
|---|---|---|---|---|
| Muscimol | Non-compendial | 28356 | μg/g | Pass |
| | in-house method by | | | |
| | HPLC-MS/MS | | | |
| Muscarine | Non-compendial | 499.453 | μg/g | Pass |
| | in-house method by | | | |
| | HPLC-MS/MS | | | |
| Ibotenic acid | Non-compendial | 14.953 | μg/g | Pass |
| | in-house method by | | | |
| | HPLC-MS/MS | | | |
| Cadmium | EPA 3052 | 0.20 | μg/g | Pass |
| Arsenic | EPA 3052 | ND μg/g | | Pass |
| Mercury | EPA 3052 | 0.14 | μg/g | Pass |
| Lead | EPA 3052 | 0.019 | μg/g | Pass |
| Stizolobinic acid | | 0.025 | μg/g | Pass |

### Example 2: Preparation of an A. muscaria Powder Extract

In this example, a ground powder extract is obtained. The same mushrooms as in Example 1 were pressed through a filter to obtain filtered extract. A total amount of 1.5 kg of mushrooms were inspected and dehydrated, and then cut into pieces having dimensions of about 1 cm×1 cm. The pH of distilled water was confirmed to be about 7 by a calibrated pH meter and the water was boiled for 10 minutes. The cut pieces of the mushrooms were placed in the boiled water. The temperature of the water was maintained at 95° C.

A fruit press was used to press the mushrooms through a filter to extract filtrate. A 1 L beaker was added to the bottom of the fruit press to collect the filtrate. Twenty-five grams of the heated mushroom pieces were added to a 125-micron filter bag having a volume of 40 liters.

The filter bag containing mushroom pieces was placed in the fruit press, and the pressure of the fruit press was increased in about 5-minute increments over the course of 1 hour to press the mushroom pieces through the filter bag. The process was repeated with a second bag containing 25 grams of the heated mushroom pieces. 14-15 liters of filtrate were collected, having a moisture content of from about 5000 to about 7000. As described herein, following filtration, the extract was acidified. The filtrate was placed in a Pyrex beaker, placed on a heating plate in a microwave oven, and heated at 100% power for 2 hours to reduce the volume to 10% of the original amount, as measured with the Pyrex beaker.

After heating, the filtrate on visual inspection was thick and colored black, and it was therefore determined that the filtrate needed to be further dried or cured. The filtrate was heated again in the microwave oven at 80% power at one-minute intervals, with stirring in between, for a total of one hour, until the color of the filtrate turned from black to light brown, demonstrating the desired change in moisture content. The resulting filtrate had a thick appearance on visual inspection and was similar in physical consistency to bread dough upon tactile inspection.

As the filtrate was cooling, filtrate was cut into pieces having a length and width of from about 3 cm to about 4 cm. Each piece was ground into a fine powder using a coffee grinder. The ground power extract was placed into a sealed plastic bag, and stored in the dark at −15° C.

Additional methods of extraction of bioactive compounds from fungal matter, methods of concentrating such extracts, and methods of standardizing such extracts and obtaining standardized preparations of one or more desired bioactive compounds therein, are known in the art, e.g., as described in CA3088384, CA3123908, and CA3124367, and references therein, and in Kondeva-Burdina, M. et al. (2019). British Industrial Biological Res. Assoc., 132, 110687.

### D. A. MUSCARIA COMPOUNDS AND ANALOGS THEREOF

In some aspects, provided herein are A. muscaria compounds, such as isolated A. muscaria compounds. In some embodiments, the isolated A. muscaria compounds are provided alone or in combination with one or more additional compounds. In some embodiments, the one or more additional compounds are A. muscaria compounds. In some embodiments, the isolated A. muscaria compounds are extracted and purified from A. muscaria fungal biomass. In some embodiments, the isolated A. muscaria compounds are provided by chemical synthesis, partial chemical synthesis (semisynthesis), or biosynthesis.

Herein, “ibotenic acid” refers to (S)-2-Amino-2-(3-hydroxyisoxazol-5-yl) acetic acid. Herein, “muscimol” refers to 5-(Aminomethyl)-1,2-oxazol-3(2H)-one. Herein, “decarboxylation” refers to a chemical reaction that removes a carboxyl group and releases carbon dioxide (CO₂), thereby replacing a carboxyl group (—COOH) with a hydrogen atom (—H); e.g., as RCO₂H→RH+CO₂. The decarboxylation reaction whereby ibotenic acid is converted to muscimol is depicted below:

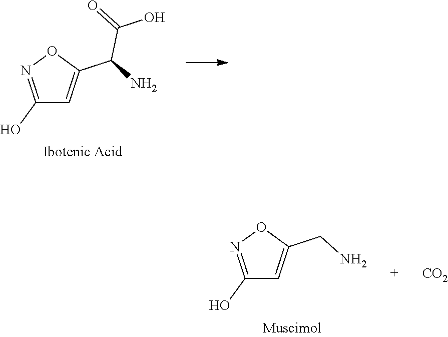 [SMILES: N[C@H](C(=O)O)c1cc(O)no1 ; NCc1cc(O)no1 ; "CO2" ; "+"]

Herein, “muscarine” refers to 2,5-anhydro-1,4,6-trideoxy-6-(trimethyl-ammonio)-D-ribo-hexitol (CAS 300-54-9), having the structure depicted below:

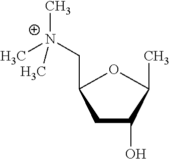

Herein, “muscazone” refers to 2-Amino-2-(2-oxo-3H-1,3-oxazol-5-yl)acetic acid (CAS 2255-39-2), having the structure below. Muscazone has been isolated from certain European specimens, and is believed to be a product of ibotenic acid breakdown by UV radiation. Muscazone has a minor pharmacological activity compared with the other agents.

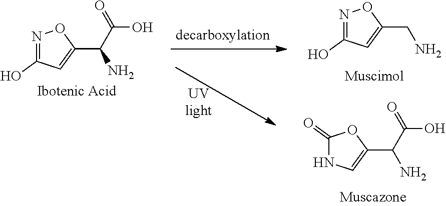 [SMILES: N[C@H](C(=O)O)c1cc(O)no1 ; NCc1cc(O)no1 ; NC(C(=O)O)c1c[nH]c(=O)o1]

a. Muscimol Analogs

In some aspects are provided analogs of muscimol. The analogs of the invention may be substituted derivatives. An analog of the invention may in particular be characterized by Formula (I) or pharmaceutically acceptable salts thereof, wherein R₁ represents H or —CH₃; R₂ represents H, an alkyl group (e.g., —CH₃, —CH₂CH₃, —CH₂CH₂CH₃, —CH(CH₃)₂, —CH(CH₃)(CH₂CH₃), or —CH₂CH(CH₃)₂), —C(O)(OCH₃), —C(O)(OCH₂CH₃), —CH₂CH₂SCH₃, phenyl, or benzyl; R₃ represents H, —CH₃, —CH₂CH₃, —CH₂CH₃, —CH₂CH₂OH, —CH₂CH₂Cl, F, Br, I, CF₃ or CN; and X represents O or S.

“Alkyl” will be understood to include radicals having any degree or level of saturation, i.e., groups having exclusively single carbon-carbon bonds, groups having one or more double carbon-carbon bonds, groups having one or more triple carbon-carbon bonds and groups having mixtures of single, double and triple carbon-carbon bonds. Preferably, an alkyl group comprises from 1 to 10 carbon atoms, and more preferably from 1 to 4 carbon atoms, inclusive.

With R₁, R₂, R₃, and X as defined above, a compound of Formula (I) is:

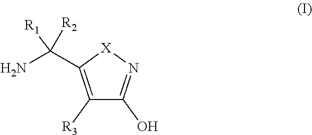

Non-limiting exemplary compounds of the invention of Formula (1) include:

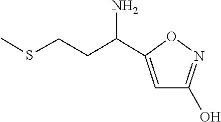

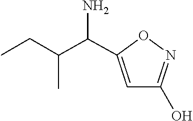

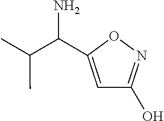

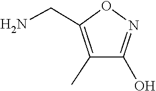

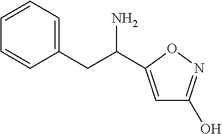 [SMILES: NC(Cc1ccccc1)c1cc(O)no1]

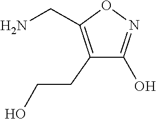 [SMILES: NCc1onc(O)c1CCO]

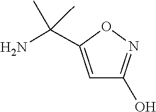

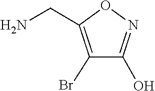

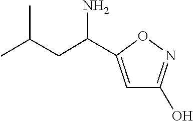

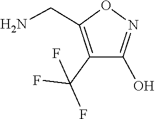 [SMILES: NCc1onc(O)c1C(F)(F)F]

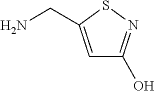

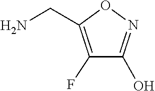

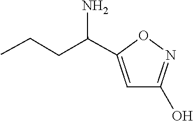

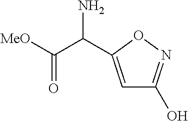 [SMILES: COC(=O)C(N)c1cc(O)no1]

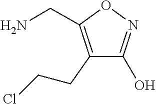 [SMILES: NCc1onc(O)c1CCCl]

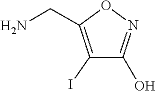

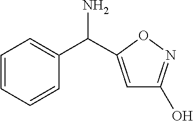 [SMILES: NC(c1ccccc1)c1cc(O)no1]

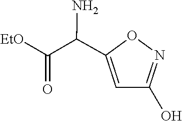 [SMILES: CCOC(=O)C(N)c1cc(O)no1]

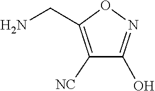

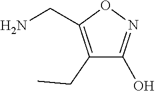

b. Muscarine Analogs

In some aspects are provided analogs of muscarine. The analogs of the invention may be substituted derivatives. An analog of the invention may in particular be characterized by Formula (II) or pharmaceutically acceptable salt thereof, wherein R₁ represents H, OH, F, O-benzyl or O-benzoyl, R₂ represents H or OH, R₃ represents H, OH, F or phenyl.

With R₁, R₂, and R₃ as defined above, a compound of Formula (II) is:

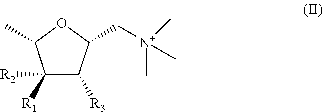

Non-limiting exemplary compounds of the invention of Formula (II) include:

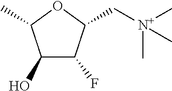

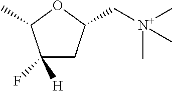

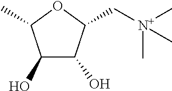

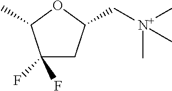

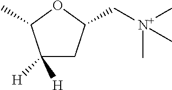

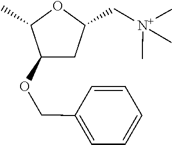

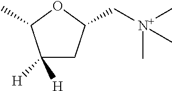

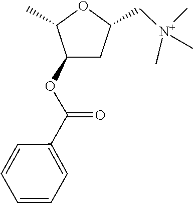 [SMILES: C[C@@H]1O[C@H](C[N+](C)(C)C)C[C@H]1OC(=O)c1ccccc1]

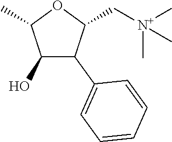

It will be readily appreciated that the disclosed compositions are not limited to a single compound, or (such as when formulated as a pharmaceutical composition) limited to a single carrier, diluent, and/or excipient alone, but may also include combinations of multiple compounds (including additional active agents), and/or multiple carriers, diluents, and excipients. Pharmaceutical compositions thus may comprise a compound together with one or more other active agents in combination, together with one or more pharmaceutically-acceptable carriers, diluents, and/or excipients, and additionally with one or more other active agents.

The disclosed compounds may contain one or more asymmetric centers and give rise to enantiomers, diastereomers, and other stereoisomeric forms. Each chiral center may be defined, in terms of absolute stereochemistry, as (R)— or (S)—. The invention includes all such possible isomers, and mixtures thereof, including racemic and optically pure forms. In some embodiments, enantiomerically enriched compounds may have an enantiomeric excess of one enantiomer of at least 50%, at least 60%, at least 65%, at least 70%, at least 75%, at least 80%, at least 85%, at least 90%, at least 95% at least 98%, at least 99% or at least 99.5%.

Optically active (R)- and (S)-, (−)- and (+)-, or (D)- and (L)-isomers may be prepared using chiral synthons or chiral reagents, or resolved using conventional techniques. When the disclosed compounds contain olefinic double bonds or other centers of geometric asymmetry, and unless specified otherwise, it is intended that the compounds include both E and Z geometric isomers. Likewise, tautomeric forms are also intended to be included.

Where disclosed compounds have at least one chiral center, they may exist as individual enantiomers and diastereomers or as mixtures of such isomers, including racemates. Separation of individual isomers or selective synthesis of individual isomers is accomplished by application of various methods that are known to those of skill. Unless otherwise indicated, all such isomers and mixtures thereof are included in the scope of the disclosed compounds.

Stereoisomers may include enantiomers, diastereomers, racemic mixtures, and combinations thereof. Such stereoisomers can be prepared and separated using conventional techniques, either by reacting enantiomeric starting materials, or by separating isomers of disclosed compounds. Isomers may include geometric isomers. Examples of geometric isomers include cis isomers or trans isomers across a double bond. Other isomers are contemplated among the compounds of the present disclosure. The isomers may be used either in pure form or in admixture with other isomers of the disclosed compounds. Various methods are known in the art for preparing optically active forms and determining activity. Such methods include standard tests described herein and other similar tests which are well known in the art.

The disclosed compounds may exist in one or more crystalline or amorphous forms. Unless otherwise indicated, all such forms are included in the scope of the disclosed compounds including any polymorphic forms. In addition, some of the disclosed compounds may form solvates with water (i.e., hydrates) or common organic solvents, including pharmaceutically acceptable solvents such as ethanol. Unless otherwise indicated, such solvates are included in the scope of the disclosed compounds. In other embodiments, the disclosed compounds exist in unsolvated form. In general, the solvated forms are considered equivalent to the unsolvated forms for the purposes of the compounds and methods provided herein.

Where the disclosed compounds contain one or more hydrogens, within the scope of said compounds are those wherein one or more of said hydrogens is replaced with a halogen. “Halogen” means any of fluoro (F), chloro (Cl), bromo (Br), or iodo (I), and any hydrogen in a compound described herein may be independently replaced with any such halogen. Where the disclosed compounds contain one or more hydrogens, within the scope of said compounds also are those wherein one or more of said hydrogens is replaced with a deuterium.

The disclosed compounds will be understood to also encompass pharmaceutically acceptable salts of such compounds. The term “pharmaceutically acceptable salt” refers to salts prepared from pharmaceutically acceptable non-toxic acids or bases, and which may be synthesized by conventional chemical methods. Generally, such salts are prepared by reacting the free acid or base forms of these agents with a stoichiometric amount of the appropriate base or acid in water or in an organic solvent, or in a mixture of the two; generally, nonaqueous media (e.g., ether, ethyl acetate, ethanol, isopropanol, or acetonitrile) are preferred. For therapeutic use, salts of the compounds are those wherein the counter-ion is pharmaceutically acceptable. Exemplary salts include those disclosed in Berge et al., “Pharmaceutical Salts,” J. Pharm. Sci. 1977; 66:1-19. Preferred salts are those employing a hydrochloride anion.

It will be appreciated that the disclosed compounds may be natural or synthetic.

Herein, “natural” refers to a compound which was isolated, extracted, or otherwise obtained from a natural source, such as a plant or fungus; while “synthetic” refers to a substance which is manufactured in a laboratory, by means of chemical synthesis, partial chemical synthesis (semisynthesis), or biosynthesis. Where compounds are not commercially available, methods for synthesis of disclosed compounds and any necessary starting materials will be as described in the art or as apparent to one of skill in view of the art (see, e.g., Loev, B., Wilson, J. W., & J Med Chem, 13(4), 738-741. https://doi.org/10.1021/jm00298a037; Green et al., “Protective Groups in Organic Chemistry,” (Wiley, 2nd ed. 1991); Harrison et al., “Compendium of Synthetic Organic Methods,” Vols. 1-8 (John Wiley and Sons, 1971-1996); “Beilstein Handbook of Organic Chemistry,” Beilstein Institute of Organic Chemistry, Frankfurt, Germany; Feiser et al, “Reagents for Organic Synthesis,” Volumes 1-17, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” John Wiley & Sons, 1995).

The disclosed compounds are generally administered as part of a pharmaceutical composition or formulation, but may be prepared for inclusion in such composition or formulations as isolated or purified compounds. The terms “isolated,” “purified,” or “substantially pure” as used herein, refer to material that is substantially or essentially free from components that normally accompany the material when the material is extracted, synthesized, manufactured, or otherwise produced or obtained. An “isolated,” “purified,” or “substantially pure” preparation of a compound is accordingly defined as a preparation having a chromatographic purity (of the desired compound) of greater than 90%, more preferably greater than 95%, more preferably greater than 96%, more preferably greater than 97%, more preferably greater than 98%, more preferably greater than 99% and most preferably greater than 99.5%, as determined by area normalization of an HPLC profile or other similar detection method. Preferably the substantially pure compound used in the invention is substantially free of any other active compounds which are not intended to be administered to a subject. In this context “substantially free” can be taken to mean that no active compound(s) other than the active compound intended to be administered to a subject are detectable by HPLC or other similar detection method, or are below a desired threshold of detection such as defined above.

### E. COMPOSITIONS

In some aspects, provided herein are compositions comprising A. muscaria extracts, A. muscaria compounds, or analogs thereof. In some embodiments, the compositions are nutraceutical compositions. In some embodiments, the compositions are pharmaceutical compositions. Pharmaceutical compositions are compositions that include the compounds together in an amount (for example, in a unit dosage form) with a pharmaceutically acceptable carrier, diluent, or excipient. It should be understood that some embodiments do not have a single carrier, diluent, or excipient alone, but include multiple carriers, diluents, and/or excipients.

Compositions can be prepared by standard pharmaceutical formulation techniques such as disclosed in Remington: The Science and Practice of Pharmacy (2005) 21th ed., Mack Publishing Co., Easton, Pa.; The Merck Index (1996) 12th ed., Merck Publishing Group, Whitehouse, N.J.; Pharm. Principles of Solid Dosage Forms (1993), Technomic Publishing Co., Inc., Lancaster, Pa.; and Ansel and Stoklosa, Pharm. Calculations (2001) 11th ed., Lippincott Williams & Wilkins, Baltimore, Md.; and Poznansky et al. Drug Delivery Systems (1980), R. L. Juliano, ed., Oxford, N.Y., pp. 253-315.

Although disclosed compositions may be referred to as “pharmaceutical” compositions or for “pharmaceutical” purpose or preparation, it will be appreciated that the term simply means that a composition is contemplated or shown to possess therapeutic or beneficial effects when administered for its intended purpose to a mammal, such as a human. It therefore will be understood that the disclosed compositions are useful regardless of the regulatory regime under which they are ultimately sold (e.g., as prescription pharmaceutical drug products or non-prescription over-the-counter (OTC) drug products, or as nutritional supplements or “nutraceuticals”), and also if not sold under a specific regulatory regime at all.

“Pharmaceutically acceptable” as used in connection with one or more ingredients means that the ingredients are generally safe and, within the scope of sound medical judgment, suitable for use in contact with the cells of humans and other animals without undue toxicity, irritation, allergic response, or complication, and commensurate with a reasonable risk/benefit ratio.

In making the disclosed compositions the active ingredients are often mixed with an excipient, diluted by an excipient, or enclosed within such a carrier which can be in the form of a capsule, sachet, paper, or other container. When the excipient serves as a diluent, it can be a solid, semi-solid, or liquid material, which acts as a vehicle, carrier, or medium for the active ingredient. Thus, the compositions can be in the form of tablets, pills, lozenges, sachets, cachets, elixirs, suspensions, emulsions, solutions, syrups, aerosols (as a solid or in a liquid medium), ointments, soft or hard gelatin capsules, suppositories, sterile injectable solutions, and sterile packaged powders. Different embodiments include immediate, delayed, extended, and controlled release forms. Many other variations are possible and known to those skilled in the art.

Examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. Formulations can additionally include: lubricating agents such as talc, magnesium stearate, and mineral oil; wetting agents; emulsifying and suspending agents; preserving agents such as methyl- and propyl-hydroxybenzoates; sweetening agents; and flavoring agents. Compositions can be formulated so as to provide quick, sustained or delayed release of the disclosed compounds after administration by employing procedures known in the art.

In preparing a formulation, it may be necessary to mill an active compound to provide the appropriate particle size prior to combining with the other ingredients. If an active compound is substantially insoluble, it may be milled to a particle size of less than 200 mesh. If an active compound is substantially water soluble, the particle size is normally adjusted by milling to provide a substantially uniform distribution in the formulation, e.g., about 40 mesh.

Pharmaceutical compositions can be formulated into any suitable dosage form, including aqueous or oil-based liquid suspensions or solutions, including tinctures; solid dosage forms, including oral solid dosage forms (e.g., tablets and capsules), sublingual or buccal tablets, confectionary products, beverage concentrates, vaporizer formulations, injectable solutions, topical formulations, transdermal formulations, controlled release formulations, fast melt formulations, delayed-release formulations, immediate-release formulations, modified release formulations, extended-release formulations, pulsatile release formulations, multi particulate formulations, and mixed immediate release and controlled release formulations. Generally speaking, one will desire to administer an amount of the compounds or extracts that is effective to achieve a plasma level commensurate with the concentrations found to be effective in vivo for a period of time effective to elicit the desired therapeutic effect(s).

In some embodiments, the disclosed compounds and extracts are formulated in a unit dosage form. The term “unit dosage form” refers to a physically discrete unit suited as unitary dosages for the subject to be treated, each unit containing a predetermined quantity of active material calculated to produce the desired therapeutic effect(s), in association with a suitable pharmaceutical carrier, diluent, or excipient. Unit dosage forms are often used for ease of administration and uniformity of dosage. Unit dosage forms can contain a single or individual dose or unit, a sub-dose, or an appropriate fraction thereof (e.g., one half a “full” dose), of the pharmaceutical composition administered. Unit dosage forms include capsules, troches, cachets, lozenges, tablets, ampules and vials, which may include a composition in a freeze-dried or lyophilized state; a sterile liquid carrier, for example, can be added prior to administration or delivery in vivo. Unit dosage forms also include ampules and vials with liquid compositions disposed therein. Unit dosage forms further include compounds for transdermal administration, such as “patches” that contact the epidermis of a subject for an extended or brief period of time.

In some embodiments, the disclosed compositions are formulated in a pharmaceutically acceptable oral dosage form, such as an oral liquid dosage form or an oral solid dosage form.

a. Oral Liquid Dosage Forms

Oral liquid dosage forms include tinctures, drops, emulsions, syrups, elixirs, suspensions, concentrates, and solutions, and the like. Oral liquid dosage forms may be formulated with any pharmaceutically acceptable excipient known to those of skill for the preparation of liquid dosage forms, and with solvents, diluents, carriers, excipients, and the like chosen as appropriate to the solubility and other properties of the disclosed A. muscaria compositions, and other ingredients. Solvents may be, for example, water, glycerin, simple syrup, alcohol, medium chain triglycerides (MCT), and combinations thereof.

Pharmaceutical formulations may be prepared as liquid suspensions or solutions using a sterile liquid, such as but not limited to, an oil, water, an alcohol, and combinations of these pharmaceutically suitable surfactants, suspending agents, emulsifying agents, may be added for oral or parenteral administration. Liquid formulations also may be prepared as single dose or multi-dose beverages, such as beverage concentrates and teas. Suspensions may include oils. Such oils include peanut oil, sesame oil, cottonseed oil, corn oil, and olive oil. Suitable oils also include carrier oils such as MCT and long chain triglyceride (LCT) oils. Suspension preparation may also contain esters of fatty acids such as ethyl oleate, isopropyl myristate, fatty acid glycerides, and acetylated fatty acid glycerides. Suspension formulations may include alcohols, (such as ethanol, isopropyl alcohol, hexadecyl alcohol), glycerol, and propylene glycol. Ethers, such as poly(ethylene glycol), petroleum hydrocarbons such as mineral oil and petrolatum, and water may also be used in suspension formulations. A suspension can thus include an aqueous liquid or a non-aqueous liquid, an oil-in-water liquid emulsion, or a water-in-oil emulsion.

In some embodiments, formulations are provided comprising the disclosed compositions and at least one dispersing agent or suspending agent for oral administration to a subject. The formulation may be a powder and/or granules for suspension, and upon admixture with water, a substantially uniform suspension is obtained. The aqueous dispersion can comprise amorphous and non-amorphous particles consisting of multiple effective particle sizes such that a drug is absorbed in a controlled manner over time.

Dosage forms for oral administration can be aqueous suspensions selected from the group including pharmaceutically acceptable aqueous oral dispersions, emulsions, solutions, and syrups. See, e.g., Singh et al., Encyclopedia of Pharm. Tech., 2nd Ed., 754-757 (2002). In addition to the disclosed A. muscaria compositions, the liquid dosage forms may comprise additives, such as one or more (a) disintegrating agents, (b) dispersing agents, (c) wetting agents, (d) preservatives, (e) viscosity enhancing agents, (f) sweetening agents, or (g) flavoring agents.

Examples of disintegrating agents for use in the aqueous suspensions and dispersions include a starch, e.g., a natural starch such as corn starch or potato starch, a pregelatinized starch, or sodium starch glycolate; a cellulose such as a wood product, microcrystalline cellulose, methylcellulose, croscarmellose, or a cross-linked cellulose, such as cross-linked sodium carboxymethylcellulose, cross-linked carboxymethylcellulose, or cross-linked croscarmellose; a cross-linked starch such as sodium starch glycolate; a cross-linked polymer such as crosspovidone; a cross-linked polyvinylpyrrolidone; alginate such as alginic acid or a salt of alginic acid such as sodium alginate; a clay; a gum such as agar, guar, locust bean, Karaya, pectin, or tragacanth; sodium starch glycolate; bentonite; a natural sponge; a surfactant; a resin such as a cation-exchange resin; citrus pulp; and sodium lauryl sulfate.

Examples of dispersing agents suitable for the aqueous suspensions and dispersions include hydrophilic polymers, electrolytes, Tween® 60 or 80, polyethylene glycol (PEG), polyvinylpyrrolidone (PVP), carbohydrate-based dispersing agents, noncrystalline cellulose, magnesium aluminum silicate, triethanolamine, polyvinyl alcohol (PVA), polyvinylpyrrolidone/vinyl acetate copolymer, poloxamers, and poloxamines.

Examples of wetting agents (including surfactants) suitable for the aqueous suspensions and dispersions include acetyl alcohol, glycerol monostearate, polyoxyethylene sorbitan fatty acid esters, PEG, oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, sodium docusate, triacetin, vitamin E TPGS, sodium taurocholate, simethicone, and phosphatidylcholine.

Examples of preservatives suitable for aqueous suspensions or dispersions include potassium sorbate, parabens (e.g., methylparaben and propylparaben) and their salts, benzoic acid and its salts, other esters of para hydroxybenzoic acid such as butylparaben, alcohols such as ethyl alcohol or benzyl alcohol, phenolic compounds such as phenol, or quaternary compounds such as benzalkonium chloride. Preservatives, as used herein, are incorporated into the dosage form at a concentration sufficient to inhibit microbial growth.

Examples of viscosity enhancing agents suitable for aqueous suspensions or dispersions include methyl cellulose, xanthan gum, carboxymethylcellulose, hydroxypropyl cellulose, hydroxypropylmethyl cellulose, Plasdone® S-630, carbomer, polyvinyl alcohol, alginates, acacia, chitosans, and combinations thereof. The concentration of the viscosity-enhancing agent will depend upon the agent selected and the viscosity desired.

Liquid formulations can also comprise inert diluents commonly used in the art, such as water or other solvents, solubilizing agents, emulsifiers, flavoring agents and/or sweeteners. Co-solvents and adjuvants also may be added to a formulation. Non-limiting examples of co-solvents contain hydroxyl groups or other polar groups, for example, alcohols, glycols, glycerol, polyoxyethylene alcohols, and polyoxyethylene fatty acid esters. Adjuvants include surfactants such as soy lecithin and oleic acid, sorbitan esters such as sorbitan trioleate, and PVP.

b. Oral Solid Dosage Forms

Oral solid dosage forms include lozenges, troches, oral thin films, softgels, tablets, capsules, caplets, powders, pellets, multiparticulates, beads, spheres, confectionery products (e.g., gummies and infused chocolates), and/or any combinations thereof. Oral solid dosage forms may be formulated as immediate release, controlled release, sustained release, extended release, or modified release formulations. In some embodiments, an oral solid dosage form is in the form of a tablet (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder), a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, or granules. In some embodiments, an oral solid dosage form is in the form of a powder. In some embodiments, the powder may be utilized as, e.g., a beverage concentrate, as a functional food, as a nutritional supplement, and as a nutraceutical powder. Additionally, formulations may be administered as a single capsule or in multiple capsule dosage form. In some embodiments, the formulation is administered in 2, 3, 4, or more capsules or tablets.

Oral solid dosage forms may contain pharmaceutically acceptable excipients such as fillers, diluents, lubricants, surfactants, glidants, binders, dispersing agents, suspending agents, disintegrants, viscosity-increasing agents, film-forming agents, granulation aid, flavoring agents, sweetener, coating agents, solubilizing agents, and combinations thereof. Oral solid dosage forms also can comprise one or more pharmaceutically acceptable additives such as a compatible carrier, complexing agent, ionic dispersion modulator, disintegrating agent, surfactant, lubricant, colorant, moistening agent, plasticizer, stabilizer, penetration enhancer, wetting agent, anti-foaming agent, alone or in combination, as well as supplementary active compound(s).

Supplementary active compounds include preservatives, antioxidants, antimicrobial agents including biocides and biostats such as antibacterial, antiviral and antifungal agents. Preservatives can be used to inhibit microbial growth or increase stability of the active ingredient thereby prolonging the shelf life of the pharmaceutical formulation. Suitable preservatives are known in the art and include EDTA, EGTA, benzalkonium chloride or benzoic acid or benzoates, such as sodium benzoate. Antioxidants include vitamin A, vitamin C (ascorbic acid), vitamin E, tocopherols, other vitamins or provitamins, and compounds such as alpha lipoic acid.

Using standard coating procedures, a film coating may be provided around the disclosed compounds (see Remington: The Science and Practice of Pharmacy (2005) 21th ed., Mack Publishing Co., Easton, Pa, supra). In one embodiment, some or all of the compounds and extracts are coated. In another embodiment, some or all of the compounds and extracts are microencapsulated. In another embodiment, some or all of the compounds and extracts are amorphous material coated and/or microencapsulated with inert excipients. In another embodiment, the compounds and extracts are not microencapsulated and are uncoated.

Suitable carriers for use in oral solid dosage forms include acacia, gelatin, colloidal silicon dioxide, calcium glycerophosphate, calcium lactate, maltodextrin, glycerin, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, hydroxypropylmethylcellulose (HPMC), hydroxypropylmethylcellulose acetate stearate (HPMCAS), sucrose, microcrystalline cellulose, lactose, and mannitol.

Suitable filling agents for use in oral solid dosage forms include lactose, calcium carbonate, calcium phosphate, dibasic calcium phosphate, calcium sulfate, microcrystalline cellulose, cellulose powder, dextrose, dextrates, dextrose, dextran, starches, pregelatinized starch, HPMC, HPMCAS, hydroxypropylmethylcellulose phthalate, sucrose, xylitol, lactitol, mannitol, sorbitol, sodium chloride, and PEG.

Suitable disintegrants for use in oral solid dosage forms include those disclosed above for aqueous suspensions and dispersions. Suitable binders impart cohesiveness to solid oral dosage form formulations. For powder-filled capsules, they aid in plug formation that can be filled into soft or hard shell capsules. For tablets, they ensure that the tablet remains intact after compression and help assure blend uniformity prior to a compression or fill step. Materials suitable for use as binders in the solid dosage forms described herein include celluloses, microcrystalline dextrose, amylose, magnesium aluminum silicate, polysaccharide acids, bentonites, gelatin, polyvinylpyrrolidone/vinyl acetate copolymer, cross-povidone, povidone, starch, pregelatinized starch, tragacanth, dextrin, a sugar (e.g., sucrose, glucose, dextrose, molasses, mannitol, sorbitol, xylitol, lactose), a natural or synthetic gum (e.g., acacia, tragacanth, ghatti gum, mucilage of isapol husks), starch, PVP, larch arabogalactan, Veegum®, PEG, waxes, and sodium alginate.

In general, binder levels of 20-70% are used in powder-filled gelatin capsule formulations. Binder usage level in tablet formulations is a function of whether direct compression, wet granulation, roller compaction, or usage of other excipients such as fillers which itself can act as moderate binders are used. Formulators skilled in the art can determine binder level for formulations. In some embodiments, a disclosed composition comprises up to 70% of one or more binder(s). Suitable lubricants or glidants for use in oral solid dosage forms include stearic acid, calcium hydroxide, talc, corn starch, sodium stearyl fumarate, alkali-metal and alkaline earth metal salts, stearic acid, sodium stearates, magnesium stearate, zinc stearate, waxes, Stearowet®, boric acid, sodium benzoate, sodium acetate, sodium chloride, leucine, PEG, methoxypolyethylene glycol, propylene glycol, sodium oleate, glyceryl behenate, glyceryl palmitostearate, glyceryl benzoate, and magnesium or sodium lauryl sulfate.

Suitable diluents for use in oral solid dosage forms include sugars (including lactose, sucrose, and dextrose), polysaccharides (including dextrates and maltodextrin), polyols (including mannitol, xylitol, and sorbitol), and cyclodextrins. Non-water-soluble diluents are compounds typically used in the formulation of pharmaceuticals, such as calcium phosphate, calcium sulfate, starches, modified starches, microcrystalline cellulose, and microcellulose (e.g., having a density of about 0.45 g/cm3, e.g., Avicel, powdered cellulose), and talc.

Suitable wetting agents for use in oral solid dosage forms include oleic acid, triethanolamine oleate, glyceryl monostearate, sorbitan monooleate, sorbitan monolaurate, polyoxyethylene sorbitan monooleate, polyoxyethylene sorbitan monolaurate, quaternary ammonium compounds (e.g., Polyquat 10®), sodium oleate, sodium lauryl sulfate, magnesium stearate, sodium docusate, triacetin, and vitamin E TPGS. Wetting agents include surfactants. Suitable surfactants for use in the solid dosage forms described herein include docusate and its pharmaceutically acceptable salts, sodium lauryl sulfate, sorbitan monooleate, polyoxyethylene sorbitan monooleate, polysorbates, poloxamers, bile salts, glyceryl monostearate, copolymers of ethylene oxide and propylene oxide, e.g., Pluronic® (BASF), and the like.

Suitable suspending agents for use in oral solid dosage forms include polyvinylpyrrolidone, PEG (having a molecular weight of about 300 to about 6000, or about 3350 to about 4000, or about 7000 to about 18000), vinylpyrrolidone/vinyl acetate copolymer (S630), sodium alginate, gums (e.g., gum tragacanth and gum acacia, guar gum, xanthans including xanthan gum), sugars, celluloses, polysorbate-80, polyethoxylated sorbitan monolaurate, polyethoxylated sorbitan monolaurate, and povidone. Suitable antioxidants for use in oral solid dosage forms include butylated hydroxytoluene (BHT), butyl hydroxyanisole (BHA), sodium ascorbate, Vitamin E TPGS, ascorbic acid, sorbic acid, and tocopherol.

Immediate-release formulations may be prepared by combining a superdisintegrant such as croscarmellose sodium and different grades of microcrystalline cellulose in different ratios. To aid disintegration, sodium starch glycolate may be added.

In cases where different agents included in a fixed-dose combination are incompatible, cross-contamination can be avoided by incorporation of the agents in different layers in the oral dosage form with the inclusion of barrier layer(s) between the different layers, wherein the barrier layer(s) comprise inert and non-functional material(s).

The above-listed additives should be taken as merely exemplary types of additives that can be included in solid dosage forms. The amounts of such additives can be readily determined by one skilled in the art, according to the particular properties desired.

Tablets can be prepared by methods well known in the art. Various methods for the preparation of the immediate release, modified release, controlled release, and extended-release dosage forms (e.g., as matrix tablets having one or more modified, controlled, or extended-release layers) and the vehicles therein are well known in the art. For example, a tablet may be made by compression or molding. Compressed tablets may be prepared by compressing, in a suitable machine, an active ingredient in a free-flowing form such as a powder or granules, optionally mixed with a binder, lubricant, inert diluent, preservative, surface-active or dispersing agent. Molded tablets may be produced by molding, in a suitable apparatus, a mixture of powdered compound moistened with an inert liquid diluent. The tablets may optionally be coated or scored and may be formulated so as to provide a slow or controlled release of the compounds and extracts. Generally recognized compendia of methods include: Remington: The Science and Practice of Pharmacy (2005) 21th ed., Mack Publishing Co., Easton, Pa; Sheth et al. (1980), Compressed tablets, in Pharm. dosage forms, Vol. 1, Lieberman & Lachtman, eds., Dekker, NY.

In some embodiments, solid dosage forms are prepared by mixing the compounds and extracts with one or more pharmaceutical excipients to form a “bulk blend” composition. The bulk blend composition is homogeneous, i.e., the compounds and extracts are dispersed evenly throughout so that the bulk blend may be readily subdivided into equally effective unit dosage forms, such as tablets, pills, and capsules. The individual unit dosages may also comprise film coatings, which disintegrate upon oral ingestion or upon contact with diluents. These formulations can be manufactured by conventional pharmaceutical techniques.

Pharmaceutical techniques for preparation of solid dosage forms include the following methods, which may be used alone or in combination: (1) dry mixing, (2) direct compression, (3) milling, (4) dry or non-aqueous granulation, (5) wet granulation, or (6) fusion. See, e.g., Lachman et al., Theory and Practice of Industrial Pharmacy (1986). Other methods include spray drying, pan coating, melt granulation, granulation, fluidized bed spray drying or coating (e.g., Wurster coating), tangential coating, top spraying, tableting, and extruding.

Compressed tablets are solid dosage forms prepared by compacting the bulk blend. In various embodiments, compressed tablets which are designed to dissolve in the mouth will comprise one or more flavoring agents. In other embodiments, the compressed tablets will comprise a film surrounding the final compressed tablet. In some embodiments, the film coating can provide a delayed release of the disclosed compounds in a formulation. In other embodiments, the film coating aids in patient compliance (e.g., flavor or sweetener coatings).

Capsules may be prepared by placing the bulk blend inside of a capsule, such as a soft gelatin capsule, a standard gelatin capsule, or a non-gelatin capsule such as a capsule comprising HPMC. The bulk blend also may be placed in a sprinkle capsule, wherein the capsule may be swallowed whole or the capsule may be opened and the contents sprinkled on food prior to eating. In some embodiments, the dose is split into multiple capsules. In some embodiments, the entire dose of the compounds and extracts is delivered in a capsule form. In some embodiments the capsule is a size 000, size 00, or size 0 soft gelatin capsule. In other embodiments, the capsule is a size 1, size 2, size 3, or size 4 soft gelatin capsule. In other embodiments, the capsule is a hard gelatin capsule of equivalent size. Capsules can be capped and packaged using a manual capsule filling machine, or automated.

In embodiments, formulations are fixed-dose pharmaceutical compositions with at least one other active agent, such as one or more of the disclosed additional active agents. Fixed-dose combination formulations may contain therapeutically efficacious fixed-dose combinations of formulations of the compounds and extracts and other active agents in the form of a single-layer monolithic tablet or multi-layered monolithic tablet or in the form of a core tablet-in-tablet or multi-layered multi-disk tablet or beads inside a capsule or tablets inside a capsule.

Depending on the desired release profile, oral solid dosage forms may be prepared as immediate release formulations, or as modified release formulations, such as controlled release, extended release, sustained release, or delayed release. In some embodiments, oral solid dosage forms are formulated as a delayed release dosage form by utilizing an enteric coating to affect release in the small intestine of the gastrointestinal tract. An enteric-coated oral dosage form may be a compressed or molded or extruded tablet/mold (coated or uncoated) containing granules, powder, pellets, beads or particles of the active ingredient and/or other composition components, which are themselves coated or uncoated. The enteric-coated oral dosage form may also be a capsule (coated or uncoated) containing pellets, beads or granules of the solid carrier or the composition, which are themselves coated or uncoated. Enteric coatings may also be used to prepare other controlled release dosage forms including extended release and pulsatile release dosage forms. Pulsatile release dosage forms may be formulated using techniques known in the art, such as described in U.S. Pat. Nos. 5,011,692, 5,017,381, 5,229,135, and 5,840,329. Other suitable dosage forms are described in U.S. Pat. Nos. 4,871,549, 5,260,068, 5,260,069, 5,508,040, 5,567,441 and 5,837,284. In one embodiment, the controlled release dosage form is pulsatile release solid oral dosage form comprising at least two groups of particles, each containing compounds and extracts described herein. The first group of particles provides a substantially immediate dose of the compounds and extracts upon ingestion by a subject. The first group of particles can be either uncoated or comprise a coating and/or sealant. The second group of particles comprises coated particles, which may comprise from about 2% to about 75%, preferably from about 2.5% to about 70%, or from about 40% to about 70%, by weight of the total dose of the compounds and extracts, in admixture with one or more binders. Using such means, a single unit dosage form can provide both a first and a second dosage amount in the single form (i.e., the first dosage amount in an immediate release form, and the second dosage amount in a delayed release form). In an embodiment, gastrorententive sustained release tablets are formulated by using a combination of hydrophilic polymer (e.g., hydroxypropyl methylcellulose), together with swelling agents (e.g., crospovidone, sodium starch glycolate, and croscarmelose sodium), and an effervescent substance (e.g., sodium bicarbonate). Using known methods, gastrorententive tablets are formulated so as to prolong gastric emptying time and extend the mean residence time (MRT) in the stomach for optimal drug release and absorption (see, e.g., Arza et al. Formulation and evaluation of swellable and floating gastroretentive ciprofloxacin hydrochloride tablets, AAPS PharmSciTech., 10(1):220-226 (2009)). Coatings for providing a controlled, delayed, or extended release may be applied to the disclosed compositions or to a core containing the compositions. The coating may comprise a pharmaceutically acceptable ingredient in an amount sufficient, e.g., to provide an extended release from e.g., about 1 hour to about 7 hours following ingestion before release of the compositions. Suitable coatings include one or more differentially degradable coatings including pH-sensitive coatings (enteric coatings), or non-enteric coatings having variable thickness to provide differential release of the disclosed compounds. Many other types of modified release systems are known to those of skill and are suitable for the formulations described herein. Examples of such delivery systems include both polymer- and non-polymer-based systems, silastic systems, peptide-based systems, wax coatings, bioerodible dosage forms, and compressed tablets using conventional binders (see, e.g., Liberman et al. Pharmaceutical Dosage Forms, 2 Ed., Vol. 1, pp. 209-214 (1990); Singh et al. Encyclopedia of Pharm. Tech., 2nd Ed., pp. 751-753 (2002); U.S. Pat. Nos. 4,327,725; 4,624,848; 4,968,509; 5,461,140; 5,456,923; 5,516,527; 5,622,721; 5,686,105; 5,700,410; 5,977,175; 6,465,014; and 6,932,983).

c. Topical and Transdermal Dosage Forms

In some embodiments, the disclosed A. muscaria compositions are provided for topical administration. In some embodiments, the disclosed A. muscaria compositions are provided for topical administration. In some embodiments, topical or transdermal administration of a disclosed A. muscaria compositions provides skin pain relief, pruritus, rejuvenation and moisturization, and immune enhancement.

In some embodiments, a disclosed A. muscaria compositions further includes a topical delivery system for topical transdermal delivery. An exemplary topical delivery system is a transdermal delivery device (“patch”) containing the active agents. Such transdermal patches may be used to provide continuous or discontinuous infusion of the compounds of embodiments of the present invention in controlled amounts. Such patches may be constructed for continuous, gradual, pulsatile, or on demand delivery of pharmaceutical agents, for example. A “patch” within the meaning of the invention may be simply a medicated adhesive patch, i.e., a patch impregnated with a pharmaceutical composition in accordance with an embodiment of the invention for application onto the skin. Thus, a patch may be a single-layer or multi-layer drug-in-adhesive patch, wherein the one or more adhesive layers also contain the active agents.

A patch may also be a “matrix” (or “monolithic”) patch, wherein the adhesive layer surrounds and overlays the drug layer (wherein a solution or suspension of the active agents is in a semisolid matrix). A “reservoir” patch may also be used, comprising a drug layer, typically as a solution or suspension of the active agents in a liquid compartment (i.e., the reservoir), separate from an adhesive layer. For example, the reservoir may be totally encapsulated in a shallow compartment molded from a drug-impermeable metallic plastic laminate, with a rate-controlling membrane made of vinyl acetate or a like polymer on one surface. A patch also may be part of a delivery system, for instance used with an electronic device communicatively coupled to the mobile device of a user, and coupled with a mobile application (e.g., to control the delivery rate from the reservoir, and optionally to provide information about delivery back to the app or user). Various transdermal patch technologies may be accordingly utilized, as known in the art.

In one example, a transdermal patch that may be used includes a self-contained module having a built-in battery that produces a low-level electric current to heat the skin and deliver a prescribed dose of a composition of an embodiment of the invention, wherein a therapeutically effective amount of the composition crosses the skin and enters the underlying tissue, so as to produce a therapeutic effect. Such a transdermal delivery device may, for example, comprise an adhesive layer, a protective film, a drug-containing reservoir (for the pharmaceutical compositions of embodiments of the invention), a heating coil, a battery, a hardware board, optionally all within a device holder, and optionally, functionally coupled to a device which is able to control drug delivery (e.g., a mobile device such as a smartphone) using a downloadable application. Such devices may, for instance, additionally shut off drug delivery automatically when a prescribed dose has been administered or may shut off automatically upon reaching a certain temperature or defined time. Such transdermal devices may be reusable or disposable.

Other release system technologies that can be used with the pharmaceutical compositions of embodiments of the invention include aromatic patches, hydrogel patches, iontophoretic patches, polyurethane patches, Geltronik patches, hyaluronic acid patches, microneedle patches, and wet wipes. Transdermal formulations in accordance with this embodiment of the invention may be applied to skin daily, such as twice to four times a day, for example, by applying a respective patch directly to the skin. Formulations for delivery through transdermal delivery devices are similar to the creams and ointments described above, except for the oily compounds for forming the creams and ointments, and the thickener for forming a gel, are not needed. Water may therefore be a sufficient excipient and the formulations can have a higher water content than the creams and appointments described above. Emulsifiers would only be required if oil based ingredients are included, such as oil-based anti-inflammatories, anti-oxidants, and/or humectants, for example. As above, the formulations may include from about 1 to about 10 micrograms per milliliter of A. muscaria extract, prepared in accordance with the Tancowny process or other processes described above or known in the art, as described above. Other amounts may be provided. Any one or more of the anti-inflammatories, antioxidants, humectants, and preservatives with respect to the creams and ointments above may be included in the transdermal formulations. In addition, a stabilizer, a solubilizer, and/or a permeation enhancing agent may be provided, as is known in the art.

Pharmaceutical compositions may be prepared as a topical dosage form. Topical dosage forms include transmucosal and transdermal formulations, such as aerosols, emulsions, sprays, ointments, salves, gels, pastes, lotions, liniments, oils, patches, and creams. For such formulations, penetrants and carriers can be included in the pharmaceutical composition. Penetrants are known in the art, and include, for transmucosal administration, detergents, bile salts, and fusidic acid derivatives. For transdermal administration, carriers which may be used include Vaseline®, lanolin, PEG, alcohols, transdermal enhancers, and combinations thereof.

In some embodiments, the disclosed compositions can be combined with other active agents, to form novel combined therapeutic formulations. Embodiments for skin care, dermatology, oral care, and cosmetic applications therefore would include skin patches, skin creams, hair loss patches, cold sore patches, mouth ulcer patches, scar reducers, hyper-hydrosis patches, skin protection patches, sun patches, protective patches, eye relax masks, pediatric teething gels, diaper sprays, menstrual pain or cramp warming patches, tension patches, anti-aging patches and masks, eye contour and eye bag patches, skin hydration patches, fat and cellulite patches, firming patches, body wraps, venopatches, nasal congestion patches, insect repellant patches, insect bite patches, foot plasters/cushions, hot/cold contrast therapy hydrogel and cold hydrogel patches, heating patches and skin wraps, and oral fluids, gels, and sprays.

In other embodiments, the disclosed compositions can be combined with other compounds such as vitamins, antioxidants, amino acids, probiotics, natural herbs or plant extracts, and other food or dietary supplements, to form patches, masks, wraps, creams, gels, oral films, or the like, for various purposes such as for energy, reduced fatigue, or improved mental and physical performance (e.g., with guarana, amino acids, and vitamins), for improved performance and reduced gastrointestinal discomfort during exercise (e.g., with L-leucine, L-isoleucine, L-valine, Vitamin B1, and Vitamin B5), for osteoporosis or function of bones and teeth (e.g., with vitamins C, D3, and K2), for sexual dysfunction, improved sexual vigor, or fertility (e.g., with vitamins, amino acids, and plant extracts such as maca root, Ginkgo biloba, Tributes terrestris, Minus pinaster, muir puma, damiana, or catuaba), for improved circulation or to sooth heavy legs (e.g., with Vitas vinifera extract and bioflavonoids), for relaxation and sleep (e.g., with melatonin, passionflower, Californian poppy, and other herbal ingredients), or to relieve muscle and joint pain or trauma, (e.g., with hemp seed oil, Arnica, and Harpagophytum).

d. Vaporizer Formulations

In some embodiments, compounds and extracts are formulated for administration by vaporization. Suitable vaporizer formulations may comprise the compounds and/or extracts described herein, a base liquid comprising any of propylene glycol (PG), vegetable glycerin (VG), polyethylene glycol (PEG), and optionally water and ethanol (where the ethanol may be an alcoholic drink or spirit, including vodka); and optionally a flavorant. In some embodiments, the optional flavorants include flavor concentrates known to those of skill, for example, flavor concentrates that replicate different food and drink flavors.

Common base liquid proportions include 50:50 (PG/VG), 30:70 (PG/VG), and 20:80 (PG/VG). However, said proportions should not be construed as limiting, as described above, the base liquid may contain anywhere from 1% to 100% PG, and/or anywhere from 1% to 100% VG. In some embodiments, one may also substitute PG or VG for polyethylene glycol (PEG), such that the base liquid comprises anywhere from 1% to 100% PEG, with the remaining base liquid, if applicable, comprising a proportion of PG and/or VG. In some embodiments, the base liquid, compounds and/or extracts, and optional flavorant may be combined via means known to those of skill in a container suitable for the resultant formulation. Once combined, the base liquid, compounds and/or extracts, and optional flavorant may be agitated to form a uniform mixture by an operator, or via paddles, arms, or other agitation means within the container.

In some embodiments, the resultant vaporizer formulation comprises compounds and/or extracts in a proportion of between 1% v/v to 50% v/v, base liquid in a proportion of between 40% v/v to 90% v/v, and optionally one or more flavorants in a proportion of between 1% v/v to 50% v/v, where each range is inclusive.

In some embodiments, the formulations may be utilized with any vaporizer device known to those of skill, including a device that is mouth-to-lung or direct-to-lung, a device that uses single-use, disposable pods; a device that uses refillable pods, a modified or “mod” pod device, including a closed pod system and an open pod system; a pen device that can be refilled with the formulations disclosed herein, including simple refillable pens, such as fixed voltage pens, vape cartridges or carts, e.g., standard 0.5 mL or 1.0 mL cartridges, variable voltage pens, and variable temperature pens; and modified pens (mods) that are custom-crafted by the user, including regulated mods (containing a circuit board) and unregulated mods (not containing a circuit board), tube mods, box mods, and mechanical mods (mechs); a disposable, single-use pen device; an e-cigarette device, a rechargeable e-cigarette device, a cigarlike device, including a disposable cigarlike and a refillable cigarlike; an e-cigar, an e-pipe, and a heat-not-burn device.

e. Additional Dosage Forms

In some embodiments, compounds and extracts are formulated for administration by injection. Formulations suitable for intramuscular, intravenous, or subcutaneous injection may comprise physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propylene glycol, polyethylene-glycol, glycerol, cremophor and the like), suitable mixtures thereof, vegetable oils (such as olive oil) and injectable organic esters such as ethyl oleate. Additionally, the disclosed A. muscaria compositions can be dissolved at concentrations of >1 mg/mL using water-soluble beta cyclodextrins (e.g., beta-sulfobutyl-cyclodextrin and 2-hydroxypropylbetacyclodextrin). Proper fluidity can be maintained, for example, by the use of a coating such as a lecithin, by the maintenance of the required particle size in the case of dispersions, and by the use of surfactants.

Formulations suitable for injection may contain additives such as preserving, wetting, emulsifying, and dispensing agents. Prevention of the growth of microorganisms can be ensured by various antibacterial and antifungal agents, such as parabens, benzoic acid, benzyl alcohol, chlorobutanol, phenol, and sorbic acid. Isotonic agents, such as sugars and sodium chloride may be used. Prolonged drug absorption of an injectable form can be brought about by use of agents delaying absorption, e.g., aluminum monostearate or gelatin.

Pharmaceutical compositions may be prepared as suspension formulations designed for extended-release via subcutaneous or intramuscular injection. Such formulations avoid first-pass metabolism, and lower dosages of the compounds will be necessary to maintain equivalent plasma levels when compared to oral formulations. In such formulations, the mean particle size of the disclosed compounds and the range of total particle sizes can be used to control the release of those agents by controlling the rate of dissolution in fat or muscle.

Pharmaceutical compositions may be prepared as nanostructured formulations such as nanoemulsions, nanocapsules, nanoparticle conjugates, or nano-encapsulated oral or nasal sprays. Preparations of such nanostructured formulations may be done by reference to the general knowledge of the art. (See, e.g., Jaiswal et al., Nanoemulsion: an advanced mode of drug delivery system, Biotech 3(5):123-27 (2015).)

The prefix “nano” as used in the terms describing various embodiments of a nanostructured formulation denotes a size range in the nanometer (“nm”) scale. Accordingly, sizes of such nanoparticle delivery vehicles include those in the range of about 1 to about 100 nm, about 100 to about 200 nm, about 200 to about 400 nm, about 400 to about 600 nm, about 600 to about 800 nm, and about 800 to about 1000 nm, as well as “microparticles” in the about 1000 to about 2000 nm (1-2 micrometer (“m”)) scale. Particles of certain sizes may be particularly advantageous depending on the method of administration (e.g., for oral liquid emulsion versus for transdermal or topical application). Regardless of method of administration, one will appreciate that smaller particles provide for increased surface area over larger particles such that a higher concentration of compounds and extracts may be applied per volume of particles. A nanoparticle may be metal, lipid, polymer or other materials, or a combination of materials, and nanoparticles may be functionalized such that another moiety also may be attached thereto. Surface functionalization may involve the use of a moiety comprising an anchor group, a spacer and/or a functional group.

Lipid-based nanoparticles (LBNPs) such as liposomes, solid lipid nanoparticles (SLN), and nanostructured lipid carriers (NLC) can be used to transport both hydrophobic and hydrophilic molecules, and can be formulated to display very low or no toxicity, and increase the time of drug action by means of prolonged half-life and controlled release of compounds and extracts. Lipid nanosystems also can include chemical modifications to avoid immune system detection (e.g., gangliosides or PEG) or to improve solubility of compounds and extracts. In addition, such nanosystems can be prepared in formulations sensitive to pH so as to promote drug release in an acid environment.

The primary components of nanoparticles are phospholipids, which are organized in a bilayer structure due to their amphipathic properties. In presence of water, they form vesicles, improving the solubility and stability of the compounds and extracts once they are loaded into their structure. Besides phospholipids, other compounds can be added to the formulations, such as cholesterol, which decreases the fluidity of the nanoparticle and increases the permeability of hydrophobic drugs through the bilayer membrane, improving stability of nanoparticles in blood. Cholesterol-modified liposomes may present a multiple bilayer with sizes from 0.5-10 nm, as multilaminar vesicles (MLVs); a single bilayer with sizes above 100 nm, as large unilamellar vesicles (LUVs); and intermediate sizes (10-100 nm), as small unilamellar vesicles (SUVs).

By way of non-limiting and merely suggestive example, the following formulations may be used in the methods of the invention, wherein “compound” refers to any one or more of the compounds of the invention (and therefore includes multiple such compounds used in combination), e.g., muscimol and/or muscarine and/or their analogs; and wherein “A. muscaria extract” is as described herein, and which may be preferably an AME-1 extract, but which will be readily understood to be merely exemplary and not limiting of the A. muscaria extracts useful in the formulations of the present invention. One of skill will readily appreciate that any number of A. muscaria extracts as can be prepared using the teachings herein in combination with the knowledge in the art, and will further understand that such extracts can be concentrated and/or standardized to create additional concentrated and/or standardized A. muscaria extracts useful in the practice of the present invention.

### Example 3: Formulation of an Aqueous or Oil Based Liquid Suspension or Solution

Liquid suspensions or solutions, with amounts per 1.0 mL are as follows:

| Ingredient | Amount |
|---|---|
| A. muscaria extract | 20 mg |
| Preservative | 3.0 mg |
| Excipient | to 1.0 mL |

Compound is mixed with an excipient, such as distilled water, an alcohol such as ethanol, or a food grade carrier oil, such as MCT oil, coconut oil, or hemp seed oil, and optionally a polyol (e.g., vegetable glycerin) and/or a lecithin (e.g., soy or sunflower lecithin). A preservative optionally will be included (e.g., sodium bisulfate, sodium citrate, and/or citric acid). Additional agents may be included if desired, e.g., flavors, sweeteners (including artificial sweaters), vitamins, active ingredients (e.g., antioxidants or anti-inflammatories), herbal extracts, essential oils, and/or any one or more of such other agents as described herein. Sufficient further excipient is then added to produce the required volume. Suspensions may be prepared in volumes of 5 mL, 10 mL, 25 mL, 30 mL, 50 mL, 100 mL, or such other total volumes as practical for research use or for sale as a pharmaceutical or OTC preparation, or a nutraceutical preparation or dietary supplement. Depending on unit dosage volume and total volume, suspensions may be used to prepare dropper bottles (e.g., 30 mL/1 oz. bottles) or fine mist spray (i.e., oral spray) bottles (e.g., 10 mL bottles). Liquid suspensions of this Example also can be used to prepare softgel capsules, ampoules, or other single unit dosage forms, through methods herein disclosed or known to those of skill.

### Example 4: Formulation of an Aqueous Liquid Solution

Water-based liquid suspensions, with amounts per 100 mL are as follows:

| Ingredient | Amount | |
|---|---|---|
| A. muscaria compound | 15 | mg |
| Melatonin | 2.5 | mg |
| L-theanine | 100 | mg |
| 5-hydroxytryptophan | 50 | mg |
| Preservative | 3.0 | mg |

| | |
|---|---|
| Excipient | to 100 mL |

Compound is mixed with water as the excipient (e.g., distilled water, deionized water, reverse osmosis or other purified water, and the like), together with one or more additional active agents for sleep, such as melatonin, L-theanine, and/or 5-hydroxytryptophan (5-HTP). A preservative optionally will be included (e.g., sodium bisulfate, sodium citrate, and/or citric acid). Additional agents may be included if desired, e.g., flavors, sweeteners, vitamins, and/or any one or more of such other agents as described herein. Sufficient further excipient is then added to produce the required volume. Suspensions may be prepared in volumes of 5 mL, 10 mL, 25 mL, 30 mL, 50 mL, 100 mL, or such other total volumes as practical for research use or for sale as a pharmaceutical or OTC preparation, or a nutraceutical preparation or dietary supplement. Depending on unit dosage volume and total volume, suspensions may be used to prepare dropper bottles (e.g., 30 mL/1 oz. bottles) or fine mist spray (i.e., oral spray) bottles (e.g., 10 mL bottles). Liquid suspensions of this Example also can be used to prepare softgel capsules, ampoules, or other single unit dosage forms, through methods herein disclosed or known to those of skill.

### Example 5: Formulation of a Liquid Suspension or Solution, Such as a Tincture

Liquid suspensions or solutions, with amounts per 1.0 mL are as follows:

| Ingredient | Amount | |
|---|---|---|
| A. muscaria compound | 10 | mg |
| Alpha lipoic acid (antioxidant) | 40 | mg |
| Sodium carboxymethyl cellulose (11%) | 12.5 | mg |
| Microcrystalline cellulose (89%) | 10 | mg |
| Sweetener | 0.35 | g |
| Sodium benzoate | 2.5 | mg |

| | |
|---|---|
| Flavor and Color | q.v. |
| Excipient | to 1.0 mL |

Compound is measured out (blended and passed through a mesh sieve if dry), and then mixed with a previously made solution of microcrystalline cellulose and sodium carboxymethyl cellulose in excipient (e.g., ethanol, for a tincture, or purified water). The sodium benzoate, flavor, and color are diluted with excipient and added with stirring. Sweetener (e.g., sucrose or sucralose) may be added if desired. Additional agents may be included, e.g., GABA or another GABA agent, vitamins, other active ingredients such as antioxidants or anti-inflammatories, herbal extracts, and essential oils. Sufficient further excipient is then added to produce the required volume. Suspensions and solutions may be prepared in volumes of 5 mL, 10 mL, 25 mL, 30 mL, 50 mL, 100 mL, or such other total volumes as practical for research use or for sale as a pharmaceutical or OTC preparation, a nutraceutical preparation or dietary supplement, or a natural product. Depending on unit dosage volume and total volume, liquid formulations may be used to prepare dropper bottles (e.g., 30 mL/1 oz. bottles) or fine mist spray (i.e., oral spray) bottles (e.g., 10 mL bottles). Liquid formulations of this Example also can be used to prepare filled softgel capsules, ampoules, or other single unit dosage forms, using methods herein disclosed or known to those of skill.

In some embodiments, the compound is any one of muscimol, muscarine, ibotenic acid, or a mixture thereof. Alternatively, 80 mg of an A. muscaria extract (including a dry or a liquid extract) may be used in place of compound, following the same procedure outlined above.

### Example 6: Formulation of Tablets

A tablet is prepared using the ingredients below:

| Ingredient | Quantity (mg/tablet) |
|---|---|
| A. muscaria compound | 12.0 |
| Cellulose, microcrystalline | 170.0 |
| Colloidal silicon dioxide | 10.0 |
| Stearic acid | 7.5 |

The ingredients are blended and compressed to form tablets. Alternatively, 125 mg of an A. muscaria extract obtained as a dry powder extract may be used in place of compound. It will be appreciated in all exemplary formulations below involving a solid form, (e.g., a tablet or capsule) even when not explicitly stated, that the extract will be obtained as a dry powder.

### Example 7: Alternate Formulation of Tablets

Scorable tablets are prepared as follows:

| Ingredient | Quantity (mg/tablet) |
|---|---|
| A. muscaria compound | 8.0 |
| Starch | 45.0 |
| Microcrystalline cellulose | 35.0 |
| PVP (as 10% solution in water) | 4.0 |
| Sodium carboxymethyl starch | 4.5 |
| Magnesium stearate | 0.5 |
| Talc | 1.0 |

Compound, starch and cellulose are passed through a No. 20 mesh U.S. sieve and mixed thoroughly. The solution of polyvinylpyrrolidone (PVP) is mixed with the resultant powders, which are then passed through a 16 mesh U.S. sieve. The granules so produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets. Tablets are scored to provide the ability to create equal half doses.

In some embodiments, the compound is any one of muscimol, muscarine, ibotenic acid, or a mixture thereof. Alternatively, an A. muscaria extract may be used in place of compound, following the same procedure outlined above.

### Example 8: Formulation of Capsules

Capsules are made as follows:

| Ingredient | Quantity (mg/capsule) |
|---|---|
| A. muscaria compound | 5.0 |
| Starch | 119.0 |
| Magnesium stearate | 1.0 |

Compound, cellulose, starch, and magnesium stearate are blended, passed through a No. 20 mesh U.S. sieve, and filled into hard or soft gelatin capsules.

In some embodiments, the compound is any one of muscimol, muscarine, ibotenic acid, or a mixture thereof. Alternatively, 50 mg of an A. muscaria extract may be used in place of compound, following the same procedure outlined above.

### Example 9: Formulation of Capsules with Additional Active Agent(s)

Capsules are made as follows:

| Ingredient | Quantity (mg/capsule) |
|---|---|
| A. muscaria compound | 3.5 |
| Cannabinoid(s) | 50.0 |
| Terpene(s) | 10.0 |
| Starch | 119.0 |
| Magnesium stearate | 1.0 |

Compound, cellulose, starch, and magnesium stearate are blended, passed through a No. 20 mesh U.S. sieve, and filled into hard or soft gelatin capsules. The cannabinoid(s) and/or terpene(s) (one or the other or both may be added, including combinations of multiple such compounds) will be as generally understood in the art.

In some embodiments, the compound is any one of muscimol, muscarine, ibotenic acid, or a mixture thereof. Alternatively, 100 mg of an A. muscaria extract may be used in place of compound, following the same procedure outlined above.

Additional active agents may include one or more cannabinoids and/or terpenes.

“Cannabinoid” refers to any one of the class of compounds that act on cannabinoid receptors or the endocannabinoid system. In some embodiments, cannabinoids include tetrahydrocannabinol (THC, including delta-9 and delta-8 THC), cannabidiol (CBD), cannabichromene (CBC), cannabidinodiol (also known as cannabinodiol) (CBND, CBDL), cannabielsoin (CBE), cannabicyclol (CBL), cannabicitran (CBT), cannabitriol (CBT), cannabivarin (CBV), cannabigerol monomethyl ether (CBGM), cannabidiphorol (CBDP), tetrahydrocannabiphorol (THCP), and iso-tetrahydrocannabinol (iso-THC), as well as their acidic forms, their propyl, methyl, and ethyl homologues, and the acid forms of those homologues forms (i.e., their acidic propyl, acidic methyl, and acidic ethyl homologue forms). Cannabinoids further include cannabinoid glycoside, acetylated, and acetylated cannabinoid forms, e.g., as described in U.S. patent application Ser. Nos. 16/110,728 and 16/110,954.

“Terpene” refers to any of the class of organic hydrocarbon isoprene polymers (isoprenoids) constituted by one or more repeating units of the five-carbon building block known as the isoprene unit (i.e., 2-methyl-1,3-butadiene, having the molecular formula C₅H₈), and including such terpenes as those structurally found in linear chains or in rings, and those having any number of isoprene units, i.e., whether as hemiterpenes (one unit), monoterpenes (two), sesquiterpenes (three), diterpenes (four), sesterterpenes (five), triterpenes (six), sesquiterpenes (seven), tetraterpenes (eight), or polyterpenes (nine or more). Terpenes are contemplated as being either derived from a botanical source, whether from Cannabis or another plant, or synthetic. In preferred embodiments, the terpenes are selected from the group consisting of alpha-bisabolol, beta-caryophyllene, camphene, carene, caryophyllene oxide, alpha-humulene, fenchol, guaiene, guaiol, limonene, linalool, myrcene, nerolidol, ocimene, alpha-phellandrene, alpha-pinene, beta-pinene, alpha-terpinene, gamma-terpinene, terpineol, and terpinolene.

### Example 10: Formulation of Capsules with Additional Active Agent(s)

Capsules are made as follows:

| Ingredient | Quantity (mg/capsule) |
|---|---|
| A. muscaria compound | 7.5 |
| Antidepressant, anxiolytic, or GABA agent | 50.0 |
| Starch | 119.0 |
| Magnesium stearate | 1.0 |

Compound, cellulose, starch, and magnesium stearate are blended, passed through a No. 20 mesh U.S. sieve, and filled into hard or soft gelatin capsules.

In some embodiments, the compound is any one of muscimol, muscarine, ibotenic acid, or a mixture thereof. Alternatively, 75 mg of an A. muscaria extract may be used in place of compound, following the same procedure outlined above.

Additional active agents may include antidepressants, anxiolytics, and/or GABA agents.

An antidepressant or anxiolytic may be any pharmaceutical agent known to act as such by one of skill (e.g., SSRIs, serotonin-norepinephrine reuptake inhibitors (SNRIs), tricyclic antidepressants (TCAs), atypical antidepressants, benzodiazepines, buspirone, etc.).

“GABA agent” refers generally to a compound that modulates the activity of a GABA receptor relative to the activity of the GABA receptor in the absence of the compound, or that otherwise elicits an observable response upon contacting a GABA receptor, including one or more subtypes. GABA agents useful in the disclosed methods include agents that modulate GABA receptor activity (as an agonist, partial agonist, antagonist, or allosteric modulator). In some embodiments, GABA receptor activity is reduced by at least about 50%, or at least about 75%, or at least about 90%. In further embodiments, GABA receptor activity is reduced by at least about 95%, or by at least about 99%. In other embodiments, GABA receptor activity is enhanced by at least about 50%, or at least about 75%, or at least about 90%. In additional embodiments, GABA receptor activity is increased by at least about 95% or at least about 99%.

Exemplary non-limiting GABA agents include (besides muscimol) baclofen, arbaclofen placarbil, bicuculline, lesogaberan, indillon, phenibut, primidone, pentetrazol, valproic acid, progabide, zaleplon, SGS-742, AZD 3353, clomethiazole, tramiprosate, gaboxadol (4,5,6,7-tetrahydroisoxazolo(5,4-c)pyridin-3-ol (THIP)), Thio-THIP, THIA, isoguvacine, adipiplon, cis-aminocrotonic acid (CACA), CGP 642103 (CAS 200402-50-2), and 1,2,5,6-tetrahydropyridine-4-yl methyl phosphinic acid (TPMPA). Additional non-limiting examples of GABA agents include those in U.S. Pat. Nos. 6,503,925; 6,218,547; 6,399,604; 6,646,124; 6,515,140; 6,451,809; and those in U.S. Patent Application Pub. Nos. 2019/0321341, 2005/0014939; 2004/0171633; 2005/0165048; 2005/0165023; 2004/0259818; and 2004/0192692; as well as such others as will be known to those of skill. In some embodiments, a GABA agent will be GABA (γ-aminobutyric acid).

### Example 11: Formulation of Capsules with Additional Active Agent(s)

Capsules are made as follows:

| Ingredient | Quantity (mg/capsule) |
|---|---|
| A. muscaria compound | 6.0 |
| Analgesic or anti-inflammatory | 125.0 |
| Palmitoylethanolamide (PEA) | 50 mg |
| Starch | 119.0 |
| Magnesium stearate | 1.0 |

Compound, cellulose, starch, and magnesium stearate are blended, passed through a No. 20 mesh U.S. sieve, and filled into hard or soft gelatin capsules.

Additional active agents may include one or more analgesics or anti-inflammatories.

An analgesic or anti-inflammatory would be any agent known to act as such (including both OTC and prescription medications) by one of skill (e.g., aspirin, acetaminophen, ibuprofen, naproxen, prescription NSAIDs, etc.).

PEA, an endogenous fatty acid amide and nuclear factor agonist shown to exert a variety of biological effects relating to chronic inflammation and pain, may be optionally included.

In some embodiments, the compound is any one of muscimol, muscarine, ibotenic acid, or a mixture thereof. Alternatively, 120 mg of an A. muscaria extract may be used in place of compound, following the same procedure outlined above.

### Example 12: Formulation of Suspension

Suspensions are made as follows:

| Ingredient | Amount | |
|---|---|---|
| Muscimol/Muscarine/Ibotenic acid | 1.8/0.36/0.013 mg | |
| Xanthan gum | 4.0 | mg |
| Sodium carboxymethyl cellulose (11%) | 50.0 | mg |
| Microcrystalline cellulose (89%) | 50.0 | mg |
| Sucrose | 1.75 | g |
| Sodium benzoate | 10.0 | mg |

| | |
|---|---|
| Flavor and color (optional) | q.v. |
| Purified water | To 5.0 mL |

The compounds, sucrose and xanthan gum are blended, passed through a No. 10 mesh U.S. sieve, and then mixed with a solution of the microcrystalline cellulose and sodium carboxymethyl cellulose in water. The sodium benzoate and optional flavor and color are diluted with water and added with stirring. Sufficient water is added to produce the required volume.

In some embodiments, 120 mg of an A. muscaria extract may be used in place of the compounds, following the same procedure outlined above.

### Example 13: Formulation of Intravenous Solution

An intravenous formulation may be prepared as follows:

| Ingredient | Amount | |
|---|---|---|
| A. muscaria compound | 500 | mg |
| Isotonic saline | 1,000 | mL |

Compound is dissolved in appropriate solvent as will be understood by those of skill; isotonic saline is used in this Example, but it will be appreciated that other solvents may be used, and additional active or inactive ingredients such as preservatives may be added, as otherwise described above, and within the general knowledge of the art.

In some embodiments, the compound is any one of muscimol, muscarine, ibotenic acid, or a mixture thereof. Alternatively, 2,500 mg of an A. muscaria extract may be used in place of compound, following the same procedure outlined above. It will be understood that the amount of the A. muscaria extract can be adjusted to reach the desired concentration (mg/mL), (as with all Examples herein).

### Example 14: Formulations of Injectable Form

Injectable formulation (e.g., for subcutaneous, intramuscular, intraperitoneal, or intravenous delivery) may be prepared as follows:

| Ingredient | Amount | |
|---|---|---|
| A. muscaria compound | 1,000 | mg |
| DMSO | 5 | mL |
| TEG | 500 | mL |

| | |
|---|---|
| Saline (1% cremaphor) | To 1 L |

Compound is dissolved in dimethyl sulphoxide (DMSO) in proportions of 1 g to 0.5 mL. Solution is brought to 37° C. and vortexed for 3-5 minutes. Tetraethyleneglycol (TEG) in the amount of 5 mL is added, and solution is returned to 37° C. and vortexed again for 3-5 mins. Solution is mixed 1:1 with saline containing 1% cremaphor to prevent precipitation. Final solution will be at 10 mg/mL active ingredients in 49.5% TEG, 49.5% saline, 0.5% DMSO, and 0.5% cremaphor. Injection may be by any suitable means, e.g., bolus injection, IV infusion, or subcutaneous infusion, for example using a drug delivery device comprising a reservoir and a pump mechanism, configured for subcutaneous administration, and which may optionally contain a user interface or be coupled to a device with a user interface such as a smartphone.

In some embodiments, the compound is any one of muscimol, muscarine, ibotenic acid, or a mixture thereof. Alternatively, 1,000 mg of an A. muscaria extract may be used in place of compound, following the same procedure outlined above.

### Example 15: Formulation of Topical Form

A topical formulation for transdermal administration may be prepared as follows:

| Ingredient | Amount (mg) |
|---|---|
| Muscimol/Muscarine/Ibotenic acid | 28/0.5/0.01 |
| Emulsifying wax | 30.0 |
| Liquid paraffin | 20.0 |
| White soft paraffin | To 100 |

The white soft paraffin is heated until molten. The liquid paraffin and emulsifying wax are incorporated and stirred until dissolved. Compounds are added and stirring is continued until dispersed. The mixture is then cooled until solid.

In some embodiments, 120 mg of an A. muscaria extract may be used in place of the compounds, following the same procedure outlined above.

It will be readily appreciated that topicals may contain any number of additional ingredients, according to the teachings herein and skill in the art. For example, in one exemplary embodiment, a topical comprises compound or an A. muscaria extract, aloe barbadensis leaf juice, parfum, Aleurites moluccanus seed oil, Simmondsia chinensis (jojoba) seed oil, polawax NF-PA-MH, Rosa canina fruit oil, Theobroma cacao (cocoa) seed butter, nigella sativa seed extract, glycerin, Hibiscus rosa-sinensis flower extract, Lavandula angustifolia (lavender) oil, citric acid, Raphanus sativus (radish) root extract.

### Example 16: Formulation of Transdermal Delivery Form

A formulation for a transdermal delivery device may be prepared as follows:

| Ingredient | Amount (mg) |
|---|---|
| Muscimol/Muscarine/Ibotenic acid | 28/0.5/0.01 |
| Permeation enhancing agent | 30.0 |
| Solubilizer | 20.0 |
| Stabilizer | To 100 |

The stabilizer, solubilizer, and permeation enhancing agent are heated and stirred until combined. Compound is added (i.e., including additional active agents, if desired) after partially cooled but before setting and stirring is continued until dispersed. The mixture is then cooled until in its desired final form (e.g., for use in a reservoir delivery system) or admixed with an adhesive and then cooled (e.g., for use in a drug-in-adhesive patch).

In some embodiments, 120 mg of an A. muscaria extract may be used in place of the compounds, following the same procedure outlined above.

### Example 17: Formulation of Topical Pain Transdermal Form

A topical formulation for transdermal administration may be prepared as follows. The emulsifiers Polawax and shea butter are provided to facilitate the mixing zinc oxide with water.

| Ingredient | Amount (%) |
|---|---|
| A. muscaria extract | 0.005% (5 micrograms/milliliter) |
| Water | 83.8% |
| Polawax | 5.0% |
| Shea butter | 3% |
| Ginkgo biloba | 0.1% |
| Zinc oxide | 8.0% |

In this example, the amount of A. muscaria extract provided in the cream is 5 micrograms/milliliter (0.00500 by volume). Any value within the range from about 1.0 to about 10.0 micrograms per milliliter (about 0.001% to about 0.01% by volume) may be provided. Glycerol may be provided instead of or along with the Polawax and shea butter as emulsifier agents, with a total emulsifier agent content of about 8.0% in this example. Aloe gel may be used instead of or along with zinc oxide, with a total combined content of about 8.0% in this example.

### Example 18: A. muscaria Formulation for Transdermal Pain

A transdermal formulation further includes an additional humectant, a fragrance, and a preservative. The additional humectant in this example is Eucalyptus oil. The fragrance in this example is coconut cream, and the preservative in this example is citric oxide. The emulsifiers polawax and shea butter further facilitate the mixing of the Eucalyptus oil and the coconut cream with the water. The cream formulation has the following formulation:

| Ingredient | Amount (%) |
|---|---|
| A. muscaria extract | 0.005% (5 micrograms/milliliter) |
| Water | 77.6% |
| Polawax | 5.0% |
| Shea butter | 3.0% |
| Ginkgo biloba | 0.1% |
| Zinc oxide | 8.0% |
| Coconut cream | 5.0% |
| Eucalyptus oil | 0.3% |
| Citric acid | 1.0% |

### Example 19: Formulation of Transdermal Anti-Pruritis and/or Anti-Inflammatory Form

A transdermal anti-pruritis and/or anti-inflammatory formulation includes Eucalyptus oil and Ginkgo biloba for their increased skin moisturization and soothing properties. Zinc oxide is provided for forming a protective barrier and for providing an anti-inflammatory effect. The cream formulation of Example 19 has the following composition by volume:

| Ingredient | Amount (%) |
|---|---|
| A. muscaria extract | 0.005% (5 micrograms/milliliter) |
| Water | 83.6% |
| Polawax | 5.0% |
| Shea butter | 3% |
| Ginkgo biloba | 0.1% |
| Eucalyptus oil | 0.3% |
| Zinc oxide | 8.0% |

As above, the amount of A. muscaria extract provided may be in a range from about 1.0 to about 10.0 micrograms per milliliter, which is equivalent to from about 0.001% to about 0.01% by volume. Glycerol may be provided instead of or along with the Polawax and shea butter as emulsifier agents, with a total emulsifier agent content of about 8.0% in this example. Aloe gel may be used instead of or along with zinc oxide, with a total content of about 8.0% in this example. An ointment could have a petrolatum content of about 61.9% petrolatum and a water content of about 21.9% for example.

### Example 20: Formulation of Transdermal Anti-Pruritis and/or Anti-Inflammatory Form

Another example of a transdermal formulation for alleviation of pruritis and/or inflammation, where moisturizers, anti-inflammatories, fragrance, and a preservative are added to the formulation of Example 19. The anti-inflammatories added in Example 20 are kukui nut oil and sea buckthor oil, which also moisturize the skin. The humectants in this example are Eucalyptus oil and Ginkgo biloba. The fragrance in this example is coconut cream fragment oil, which also moisturizes the skin. The emulsifiers polawax and shea butter are provided to facilitate the mixing of the water soluble and oil soluble components. The preservative in this example is citric oxide. The cream formulation has the following composition by volume:

| Ingredient | Amount (%) |
|---|---|
| A. muscaria extract | 0.005% (5 micrograms/milliliter) |
| Water | 57.9% |
| Polawax | 5.0% |
| Shea butter | 3.0% |
| Ginkgo biloba | 0.1% |
| Eucalyptus oil | 0.3% |
| Zinc oxide | 8.0% |
| Kukui nut oil | 10.0% |
| Sea buckthorn oil | 10.0% |
| Coconut cream fragment oil | 5.0% |
| Citric acid | 1.0% |

### Example 21: Formulation of a Transdermal Rejuvenation/Moisturizing Form

A topical rejuvenation/moisturizing formulation is a transdermal formulation that includes aloe vera, cocoa seed butter, and glycerin for their moisturizing qualities. Aloe vera and Hibiscus also have anti-inflammatory qualities. Glycerin acts as an emulsifier, along with polawax and shea butter. Citric acid is provided as a preservative. Example 21 has the following composition by volume:

| Ingredient | Amount (%) |
|---|---|
| A. muscaria extract | 0.005% (5 micrograms/milliliter) |
| Water | 79.0% |
| Polawax | 5.0% |
| Shea butter | 3.0% |
| Aloe vera | 5.0% |
| Cocoa seed butter | 1.0% |
| Glycerin | 5.0% |
| Hibiscus | 1.0% |
| Citric acid | 1.0% |

The amount of A. muscaria extract provided may be in a range from about 1.0 to about 10.0 micrograms per milliliter, which is equivalent to from about 0.001% to about 0.01% by volume. Glycerol may be provided instead of or along with the Polawax and shea butter as emulsifier agents to facilitate the mixing of the water soluble and oil soluble components, with a total emulsifier agent content of about 8.0% in this example. Aloe gel may be used instead of or along with zinc oxide, with a total content of about 8.0% in this example.

### Example 22: Formulation of a Transdermal Rejuvenation/Moisturizing Form

A transdermal rejuvenation/moisturizing formulation that further includes the anti-inflammatories black cumin, Aleurites molusccanus Seed Oil, jojoba oil, Rosa canina, and sativa seed extract. Lavender is also included, which is both anti-inflammatory and a fragrance. Example 22 has the following composition by volume:

| Ingredient | Amount (%) |
|---|---|
| A. muscaria extract | 0.005% (5 micrograms/milliliter) |
| Water | 78.4% |
| Polawax | 5.0% |
| Shea butter | 3.0% |
| Black cumin | 10% |
| Aloe vera | 5.0% |
| Cocoa seed butter | 1.0% |
| Glycerin | 5.0% |
| Jojoba oil | 5.0% |
| Aleurites molusccanus seed oil | 2.0% |
| Sativa seed extract | 1.0% |
| Rosa canina | 0.65% |
| Hibiscus | 1.0% |
| Parfum | 2.0% |
| Lavender | 2.0% |
| Radish root | 1.0% |
| Citric acid | 1.0% |

### Example 23: Formulation of a Transdermal Rejuvenation/Moisturizing Form

A transdermal rejuvenation/moisturizing formulation based on Example 22 that further includes aloe vera, cocoa seed butter, and glycerin for their moisturizing qualities. Aloe vera and Hibiscus also have anti-inflammatory qualities. Glycerin also acts as an emulsifier. Citric acid is provided as a preservative. The transdermal formulation of Example 23 has the following composition by volume.

| Ingredient | Amount (%) |
|---|---|
| A. muscaria extract | 0.005% (5 micrograms/milliliter) |
| Water | 78.0% |
| Polawax | 5.0% |
| Shea butter | 3.0% |
| Aloe vera | 5.0% |
| Cocoa seed butter | 1.0% |
| Glycerin | 5.0% |
| Hibiscus | 1.0% |
| Citric acid | 1.0% |

As above, the amount of A. muscaria extract provided may be in a range from about 1.0 to about 10.0 ug per mL, which is equivalent to from about 0.001% to about 0.01% by volume. Glycerol may be provided instead of or along with Polawax and shea butter as emulsifier agents, with a total emulsifier agent content of about 8.000 in this example. Aloe gel may be used instead of or along with zinc oxide, with a total content of about 8.0% in this example.

### Example 24: Formulation of Transdermal Immune Enhancement Form

A transdermal immune enhancement formulation includes cat's claw, Hibiscus, and nettle leaf for their immune enhancing characteristics. Black cumin is provided for its anti-inflammatory effect. Citric acid is provided as a preservative. The cream formulation of Example 24 has the following composition by volume:

| Ingredient | Amount (%) |
|---|---|
| A. muscaria extract | 0.005% (5 micrograms/milliliter) |
| Water | 78.8% |
| Polawax | 5.0% |
| Shea butter | 3.0% |
| Black cumin | 10.0% |
| Cat's claw | 50 mg |
| Hibiscus | 1.0% |
| Nettle leaf | 1.0% |
| Citric acid | 1.0% |

As in the above formulations, the amount of A. muscaria extract provided in a range from about 1.0 to about 10.0 micrograms per milliliter, which is equivalent to from about 0.001% to about 0.01% by volume. The A. muscaria extract is about 0.005% (5 micrograms/milliliter). Glycerol may be provided instead of or along with the Polawax and shea butter as emulsifier agents, with a total emulsifier agent content of about 8.0% in this example.

### Example 25: Formulation of Transdermal Immune Enhancement Form

A transdermal immune enhancement formulation based on Example 24 and further includes the anti-inflammatories moringa oil and spearmint oil. Aloe vera is included to provide a protective layer and for moisturization of the skin. Parfum is also included as a fragrance. Example 25 has the following composition by volume:

| Ingredient | Amount (%) |
|---|---|
| A. Muscaria extract | 0.005% (5 micrograms/milliliter) |
| Water | 68.7% |
| Polawax | 5.0% |
| Shea butter | 3.0% |
| Black cumin | 10.0% |
| Cat's claw | 50 mg |
| Hibiscus | 1.0% |
| Nettle leaf | 1.0% |
| Moringa oil | 1.0% |
| Aloe vera | 5.0% |
| Ginkgo biloba | 0.1% |
| Spearmint oil | 2.0% |
| Parfum | 2.0% |
| Citric acid | 1.0% |

As above, the amount of A. muscaria extract provided may be in a range from about 1.0 to about 10.0 micrograms per milliliter, which is equivalent to from about 0.0010% to about 0.010% by volume. Glycerol may be used instead of or along with the Polawax and shea butter as emulsifier agent(s), with a total emulsifier agent content of about 8.000 in this example.

The active ingredient(s) are added (i.e., including additional active agents, if desired) after partially cooled but before setting and stirring is continued until dispersed. The stabilizer, solubilizer, and/or permeation enhancing agent, if provided, are added and stirred until combined. The mixture is then cooled until in its desired final form (e.g., for use in a reservoir delivery system) or admixed with an adhesive and then cooled (e.g., for use in a drug-in-adhesive patch).

### Example 26: Formulation of Cut Matrix Sublingual or Buccal Tablets

Sublingual or buccal tablets are made as a single matrix and then cut to size:

| Ingredient | Amount (mg/tablet) |
|---|---|
| Muscimol/Muscarine/Ibotenic acid | 28/0.5/0.01 |
| Glycerol | 210.5 |
| Water | 143.0 |
| Sodium citrate | 4.5 |
| Polyvinyl alcohol | 26.5 |
| Polyvinylpyrrolidone | 15.5 |

The glycerol, water, sodium citrate, polyvinyl alcohol, and polyvinylpyrrolidone are admixed together by continuous stirring and maintaining the temperature at about 90° C. When the polymers have gone into solution, the solution is cooled to about 50-55° C. and compound is slowly admixed. The homogenous mixture is poured into forms made of an inert material to produce a drug-containing diffusion matrix having a thickness of about 2-4 mm. This diffusion matrix is then cut to form individual tablets having the appropriate size.

In some embodiments, 120 mg of an A. muscaria extract may be used in place of the compounds, following the same procedure outlined above.

### Example 27: Formulation of Individually Formed Sublingual or Buccal Lozenges

Sublingual or buccal lozenges are made from individual forms or molds:

| Ingredient | Amount (mg/each lozenge) |
|---|---|
| Muscimol/Muscarine/Ibotenic acid | 28/0.5/0.01 |
| Silica gel powder | 350 |
| Citric acid powder | 400 |
| Acacia powder | 600 |
| Flavor (optional) | 100 |
| Polyethylene glycol | 1,000 |

The inactive ingredients are admixed by continuous stirring and maintaining the temperature at about 90° C. When the PEG has melted and the other ingredients have gone into solution, the solution is cooled to about 50-55° C. and compound is slowly admixed. The homogenous mixture is poured into separate molds and allowed to cool. Reference may also be made to U.S. Pat. No. 10,034,832 and the Examples therein, all of which is incorporated herein.

In some embodiments, 120 mg of an A. muscaria extract may be used in place of the compounds, following the same procedure outlined above.

### Example 28: Formulation of Confectionery Products and Gummies

Confectionery products such as chewy “gummies” can be made as follows:

| Ingredient | Amount (mg/each gummy) |
|---|---|
| Muscimol/Muscarine/Ibotenic acid | 28/0.5/0.01 |
| Sugar | 675 |
| Corn syrup 42 DE | 1,000 |
| Fruit juice concentrate (65-70% solids) | 200 |
| Distilled or deionized water | 175 |
| Palm kernel oil | 150 |
| Gelatin (250 bloom) | 40 |
| Citric acid | 25 |
| Malic acid | 7.5 |
| Flavoring | 7 |
| Monoglyceride | 4 |
| Lecithin | 3 |
| Colorants | 2 |

The gelatin is dissolved in water and added to a mixer. Next, a syrup is made in a vacuum cooking system by continuously mixing and cooking sugar, water, and the corn syrup to a temperature of about 250° F.-275° F. The sugar, corn syrup and other liquids are cooked in a jacketed mixing kettle with an agitator to a temperature of about 125° F.-150° F. and cooked to a final temperature of about 250° F.-275° F. in a vacuum cooking system. This syrup is added to the dissolved gelatin in the mixer and is blended until the mixture thickens (for about 3 minutes). The mixture is then blended at high speed until it is aerated (for about 2 minutes). Next, the mixer speed is lowered and the remaining ingredients are added to form a paste, which is mixed until homogenous (for about 2 minutes). The homogenous mixture is pumped out of the mixer, and then formed into desired shapes by using a puller unit, a batch roller, rope sizers, and/or a die former (drop roller or ball former). Other such products, including candy coated confectionery products, are disclosed in U.S. Pub. No. 2005/0191406, which is incorporated by reference.

Alternatively, 250 mg of an A. muscaria extract may be used in place of compound, following the same procedure outlined above.

### Example 29: Vaporizer Formulation

50:50 base liquid vaporizer formulation comprising 50 mg/mL compound can be prepared as follows:

| Ingredient | Quantity (units) | |
|---|---|---|
| A. muscaria extract | 500 | mg |
| 50:50 base liquid (50% PG, 50% VG) | 10 | mL |

The ingredients are mixed and prepared for use with any liquid vaporization device or appliance, such as e-liquid vaporizers, e-cigs, mods, vape pens, and the like, and can be formulated for any other oil, thin oil, “e-juice,” or e-liquid vaporizer, according to ordinary skill. Flavoring(s) optionally may be added if desired, and water and/or ethanol may be added to the base liquid as a diluent.

### Example 30: Alternative Vaporizer Formulation

50:50 base liquid vaporizer formulation comprising 1000 A. muscaria extract can be prepared as follows:

| Ingredient | Percentage v/v |
|---|---|
| A. muscaria extract | 10% |
| 50:50 base liquid (50% PG, 50% VG) | 90% |

It will be appreciated that the above example may be produced in any desired volume, and the ingredients may be combined with tools known to those in the art. As an example, if the desired volume is 10 mL, 4.5 mL PG, 4.5 mL VG, and 1 mL A. muscaria extract will be combined. Alternatively, one may include in the base liquid any of PEG, ethanol, and water, depending on the specific desired embodiment.

### Example 31: Formulation of Intranasal Delivery Form

A nasal spray formulation for intranasal delivery may be prepared as follows:

| Ingredient | Quantity (units) | |
|---|---|---|
| A. muscaria extract | 500 | mg |
| DMSO | 50 | μL |
| MCT | 5 | mL |

| | |
|---|---|
| Saline (1% cremaphor) | To 10 mL |

The solution at 10 mg/mL of active ingredients in 49.5% MCT, 49.5% saline, 0.5% DMSO, and 0.5% cremaphor is prepared, as above (but with MCT in place of TEG), for use in nasal spray device. In other embodiments, a nasal formulation can be prepared as a dry powder for inhalation, e.g., by combining compound with lactose and mixing for use with a dry powder inhaling appliance, or as in U.S. Pub. No. US2015/0367091A1 and references cited therein.

### Example 32: Formulation of Liquid or Dry Powder Beverage Concentrate

A formulation suitable for use as a beverage concentrate powder is made as follows:

| Ingredient | Quantity (mg) |
|---|---|
| A. muscaria extract | 870.0 |
| Glycerin | 55.0 |
| Sweetener | 40.0 |
| Flavorings | 2.0 |
| Colorants | 1.0 |
| Salt | 0.5 |
| Stabilizing gum | 0.3 |
| Preservatives | 0.2 |

Formulations are prepared with ingredients admixed and blended until consistent, the above ingredients being merely exemplary. Optional additional components include further flavorings and colorants, further preservatives, acidulants, and emulsifying agents. The limitations on the additions of all of these optional components are variable dependent on their final effect on taste, mouthfeel, and viscosity, which they should not adversely affect in the final beverage product. In some embodiments, the overall viscosity of the liquid beverage concentrate should remain sufficiently low to enable the use of conventional beverage pumps and dispensers.

In some embodiments, a final beverage product will be created by mixing the concentrate with between about two and about 20 volumes of water, or more as will vary by personal preference. In alternative embodiments, a concentrate can be added to another liquid such as juice, tea, soda, a sports or electrolyte drink, a cocktail or alcoholic beverage, or the like.

Concentrates can also be prepared and added to prepackaged beverages such as teas, juices, water, sports drinks and electrolyte drinks, single-serving beverage pouches and “shots,” alcoholic beverages, and the like, and whether for a single or multiple serving (it also will be readily appreciated that in other embodiments, compound can be added directly to any beverage without additional preparation or formulation).

### Example 33: Formulation of Syrup

Syrup containing an A. muscaria extract can be made as follows:

| Ingredient | Quantity (units) | |
|---|---|---|
| A. muscaria extract | 2 | g |
| Filtered water | 750 | mL |
| Fine granulated sugar | 750 | mL |
| Vegetable glycerin | 30 | mL |

The water and sugar are brought to the boil, and stirred consistently, until fully dissolved. The temperature is reduced to a simmer and the A. muscaria extract is added, then stirred until fully blended into the mixture. Vegetable glycerin is added. Mixture is simmered for 5 to 6 minutes, and stirred briefly and briskly at each minute. The hot syrup is passed through a No. 20 mesh U.S. sieve into a container. The resulting syrup is allowed to cool, and can be added to beverages or used in baking.

### Example 34: Formulation of Elixir

Elixir is made as follows:

| Ingredient | Quantity (units) | |
|---|---|---|
| A. muscaria extract | 10 | g |
| Coconut oil | 177 | mL |
| Unrefined vegetable oil (e.g., hemp seed oil) | 532 | mL |
| Lecithin liquid | 15 | mL |
| Grain alcohol (190 proof/95% ethanol). | 355 | mL |

The A. muscaria extract is placed in a boiling water bath. The coconut oil is melted and mixed with A. muscaria extract in a crucible. Lecithin liquid is added to the crucible to form a mixture. Unrefined vegetable oil is added to the mixture and mixing to form the Cannabis elixir. The elixir prepared using the techniques described above may be used topically, orally as a tincture, or in pill form. To double the strength of the elixir, the quantity of the oils and lecithin may be reduced to half. The elixir should be brilliantly clear and can be strained or filtered, or further clarified through the addition of purified talc or siliceous earth. Sorbitol, glycerin, sucrose or artificial sweeteners may also be used to sweeten the mixture. An adjuvant solvent (propylene glycol) may be added to reach a desired volume.

### Example 35: Formulation of Oral Thin Film

An oral thin film is made as follows:

| Ingredient | Quantity (100 mg thin film) | |
|---|---|---|
| A. muscaria extract | up to about 65 wt. % | |
| Purified water | 2-24 | wt. % |
| Pectin | 4-50 | wt. % |
| Vegetable oil | 0-22 | wt. % |
| Glycerin | 3-22 | wt. % |
| Microcrystalline cellulose | 0-20 | wt. % |
| Sodium benzoate | 0-0.1 | wt. % |
| Flavoring agent | 0-10 | wt. % |
| Coloring agent | 0-1.0 | wt. % |

The vegetable oil, glycerin and purified water are blended to provide a uniform first mixture. The mixing will typically be carried out at an elevated temperature (e.g., about 130-140° F. or 54.4-60° C.) The mixing can be carried out in any suitable manner, employing equipment known to those of skill, e.g., blending in a blender. The A. muscaria extract is then added to the mixture at room temperature (e.g., about 70° F.) for a suitable period of time (e.g., up to about 5 minutes) sufficient to provide the thickened second mixture. Pectin, microcrystalline cellulose, flavoring agents, and sweetening agents are then added, thickening the second mixture, creating a slurry. The slurry is cooled, sheared, mixed, cast, and condensed to provide an oral thin film, which can then be cut. Alternatively, the slurry is hot extruded, cast, and condensed to provide an oral thin film, which can then be cut. Alternatively, the vegetable oil can be replaced with other lipids such as deodorized cocoa butter oil or fruit seed oil. Similarly, glycerin can be replaced with ethoxylated monoglycerides or ethoxylated diglycerides. The thin film described herein can optionally further include a mucoadhesive agent. The mucoadhesive agent, when placed in the oral cavity in contact with the mucosa therein, adheres to the mucosa. Alternatively, up to about 10 wt. % of an A. muscaria compound may be used in place of A. muscaria extract, following the same procedure outlined above.

### Example 36: Formulation of a Troche

A single troche is made as follows:

| Ingredient | Quantity (mg per troche) |
|---|---|
| A. muscaria extract | 880 |
| Polyethylene glycol 1450 | 1000 |
| Stevia | 100 |
| Acacia gum | 600 |
| Citric acid | 400 |

A base is prepared by melting polyethylene glycol 1450, with stevia, acacia gum, and citric acid at a temperature between 58° C. and 64° C. at normal atmospheric pressure. An A. muscaria extract is then added to form a uniform mixture, with the temperature maintained between approximately 58° C. and 63° C. The resultant solution is then poured into a lozenge or troche mold device, using a micropipette. The mixture is allowed to cool at room temperature.

### Example 37: Formulation of a Caplet

A single caplet is made as follows:

| Ingredient | Quantity (mg/caplet) |
|---|---|
| A. muscaria extract | 880.0 |
| Starch | 45.0 |
| Microcrystalline cellulose | 30.0 |
| Magnesium stearate | 0.5 |
| Silicon dioxide | 10.0 |
| Acacia gum | 2.0 |

A. muscaria extract, starch, magnesium stearate, silicon dioxide and acacia gum are blended and passed through a No. 20 mesh U.S. sieve. High pressure is applied to the powder bed, compressing the powder into a coherent compact. Additional excipients such as sucrose, talc, or titanium dioxide, may also be used to coat the caplet.

### Example 38: Formulation of a Functional Food

In some embodiments, the disclosed A. muscaria compositions are used as functional foods. In some embodiments, the disclosed A. muscaria compositions are used in the manufacture of a functional food. In some embodiments, the disclosed A. muscaria compositions are administered to a subject as a functional food. In some embodiments, a functional food comprises a disclosed A. muscaria extract. In some embodiments, a functional food comprises a disclosed A. muscaria compound, an analog thereof, or any combination thereof. In some embodiments, a functional food provides the benefits described herein to a subject who ingests said functional food.

In some embodiments, the functional food comprising the disclosed A. muscaria compositions is a dairy product. Exemplary dairy products include milk, yogurt, and cream cheese. In some embodiments the functional food comprising the disclosed A. muscaria compositions is a grain product. Exemplary grain products include bread, noodles, and cereal.

One exemplary functional food containing A. muscaria extract is as follows:

| | Quantity |
|---|---|
| Ingredient | (per powdered serving) |
| A. muscaria extract | 880 mg |
| Carbohydrate source (e.g., FOS) | 40-80% |
| Fat (e.g., coconut oil) | 20-60% |
| Protein (e.g., casein) | 1-10% |
| Emulsifier (e.g., mono or diglycerides) | 1-2% |
| Stabilizer (e.g., sodium polyphosphate) | 2-3% |
| Free flowing agent (e.g., silicon dioxide) | 0.5-1% |
| Water | Calculated & added to |
| | achieve solid total of 60-72% |

All ingredients, except the free flowing agent, are added and blended using a homo-mixer to provide an emulsion with a solid total of 60-72%. FOS refers to fructo-oligosaccharides. Remixing is performed to produce a more uniform emulsion mixture by passing the mixture through homogenized pressure of between 150-250 bars. Sterilization is performed to kill microbes. The mixture is pasteurized at a temperature of 75-90° C. for at least 30 seconds. The mixture is then pumped. The mixture is then dried to evaporate the water content to obtain the final product in the form of a powder. The drying process is carried out using a spray dryer unit with inlet air temperature of 150-200° C. and the discharge air temperature is 88-95° C. During the drying process the moisture content of the product is maintained at 1.5% to 5.0%. Finally, the powder is sieved to create a product with a uniform grain. During the sieving process, a free-flowing agent is added. The functional food can be consumed directly by dissolving 10 grams of powder into 100 mL of hot water with a temperature of 85±5° C. In addition to direct consumption, functional foods can be added to drinks or processed food products. The product is prepared so that a single serving size comprises 880 mg of the A. muscaria extract.

### Example 39: Formulation of an Effervescent Powder (Wet and Dry)

An effervescent powder is made as follows:

| Ingredient | Quantity (w/v %) |
|---|---|
| A. muscaria extract | 20.0 |
| Citric acid | 12.0 |
| Tartaric acid | 24.0 |
| Sodium hydrogen carbonate | 41.0 |
| PVP30 | 3.0 |

In order to prepare the effervescent mixture, the alkaline component and acid component are prepared in a stoichiometric ratio, in which the alkaline component is present in a stoichiometric excess to that of the acid component. An A. muscaria extract (and any additional active agent) is incorporated therein and dry blended. Optional flavorings, sweeteners, preservatives, stabilizers and antioxidants, if desired, are incorporated at this point. Alternatively, if a wet granulation is performed, a solution containing a selected binder and solvent is prepared and added to the mixture. The preparation is mixed until suitable granulation is achieved. Flavorants, sweeteners, preservatives and antioxidants, if added, are incorporated in the binder/solvent solution. The granules are then dried, milled, and screened to the desired size. The quantity of A. muscaria extract included in the effervescent formulation may range from about 10% to about 65% by weight. Suitable bicarbonates appropriate for the formulation of the present invention include sodium bicarbonate, calcium bicarbonate, potassium bicarbonate, barium bicarbonate, strontium bicarbonate, and magnesium bicarbonate. Other acids which may be used include citric, ascorbic, malic, succinic, phosphoric, and monopotassium phosphate. Organic acids such as citric, tartaric and malic acids are particularly suitable.

### Example 40: Formulation of a Beverage

In some embodiments, the disclosed A. muscaria compositions are used as a beverage, such as a drinkable liquid. In some embodiments, the disclosed A. muscaria compositions are used in the manufacture of a beverage. In some embodiments, the disclosed A. muscaria compositions are administered to a subject as a beverage. In some embodiments, the disclosed A. muscaria compositions are provided as a powder, e.g., a beverage powder for mixing with a liquid. In some embodiments, the disclosed A. muscaria compositions are provided as a ready to drink beverage. In some embodiments, a ready to drink beverage is provided as a packaged beverage, such as in a prepared form, ready for consumption. In some embodiments, the beverage provides any one or more of the benefits described herein to a subject who ingests said beverage. In some embodiments, the beverage comprising a disclosed A. muscaria composition is prepared from a beverage powder. In some embodiments, the beverage powder comprises a disclosed A. muscaria composition, e.g., an extract, compound, and analog thereof. The beverage powder can be added to a variety of liquids to introduce the nutritional benefits of a disclosed A. muscaria composition to the beverage. In some embodiments, the beverage powder comprising a disclosed A. muscaria composition is added to a beverage comprising dairy. In some embodiments, the beverage powder comprising a disclosed A. muscaria composition is added to another powder comprising dairy. Examples of dairy beverages, whether in liquid or powder form, include milk, chocolate milk, or hot chocolate. In some embodiments, the beverage powder comprising a disclosed A. muscaria composition is added to a beverage comprising protein. In some embodiments, the beverage powder comprising a disclosed A. muscaria composition is added to another powder comprising protein, such as a protein powder.

In some embodiments, the beverage comprising a disclosed A. muscaria composition is a ready-to-drink beverage. In some embodiments the disclosed A. muscaria compositions will be incorporated into a ready-to-drink beverage. A ready-to-drink beverage is a beverage sold in a prepared form ready for consumption. Exemplary benefits provided by a ready to drink beverage comprising an A. muscaria composition include improvements in endurance and strength, promotion of calmness, relief from muscle soreness, and achievement of spiritual enlightenment. Improvements in such areas are described herein, such as above. In some embodiments the ready-to-drink beverage comprising the disclosed A. muscaria compositions is a dairy beverage. Examples of ready-to-drink dairy beverages include malted milk, chocolate milk, or strawberry milk. In some embodiments the ready-to-drink beverage comprising disclosed A. muscaria compositions is an herbal beverage. Examples of ready-to-drink herbal beverages include black tea, chamomile tea, or green tea. In some embodiments the ready-to-drink beverage comprising disclosed A. muscaria compositions is a carbonated beverage. Examples of ready-to-drink carbonated beverages include cola, carbonated water, or ginger ale. In some embodiments the ready-to-drink beverage is a fruit-based beverage. Examples of ready-to-drink fruit-based beverages include lemonade, smoothies, or fruit juice. In some embodiments the ready-to-drink beverage comprising disclosed A. muscaria compositions is an alcoholic beverage. Examples of ready-to-drink alcoholic beverages include beer, hard cider, or a cocktail.

An herbal or medicinal tea comprising an A. muscaria extract is made as follows:

| Ingredient | Quantity |
|---|---|
| A. muscaria extract | 2 g |
| Additional herbal agents | 2 g |

An A. muscaria extract and any additional dried herbal or medicinal agents are mixed together. Additional herbal agents appropriate for inclusion in tea blends may include medicinal mushrooms (i.e. chaga, Lion's Mane, or reishi), peppermint, tulsi, gotu kola, Ginkgo biloba, rosemary, lemon balm, passionflower, skullcap, chamomile, oatstraw, spearmint, lavender, rose, valerian, licorice, dandelion root, milk thistle, schisandra, ginger, cacao nibs, chicory root, or Stevia. The active agents are then inserted into a tea bag. In an alternative embodiment, the herbal or medicinal tea may follow a loose-leaf formulation. Dried A. muscaria extract and dried additional herbal agents are steeped directly in one cup of hot water for 10-15 minutes. The mixture is confined to a tea strainer as it is steeping, or strained after it has steeped.

### Example 41: Formulation of Nutritional Supplement

A. muscaria extract dietary supplements are made as follows:

| Ingredient | Quantity (mg/capsule) | |
|---|---|---|
| A. muscaria extract | 440 | mg |
| Cellulose | 119.0 | mg |
| Magnesium stearate | 1.0 | mg |

A. muscaria extract and magnesium stearate are blended, passed through a No. 20 mesh U.S. sieve, and inserted into a hard cellulose capsule. Additional active ingredients such as herbs, vitamins, enzymes, amino acids, prebiotics, probiotics, or minerals may also be added at the same time as the A. muscaria extract and magnesium stearate. Two capsules may be administered to a subject or self-administered by a subject for a total of 880 mg of extract.

### Example 42: Formulation of a Nutritional Softgel Supplements

A. muscaria extract nutritional softgel supplements are made as follows:

| Ingredient | Quantity (units) | |
|---|---|---|
| A. muscaria extract | 500 | mg |
| Potato starch | 10 | wt. % |
| Gelatin (170-180 bloom) | 34 | wt. % |
| Purified water | 26 | wt. % |
| Glycerin (99.5%) | 28 | wt. % |
| Oil | 1.5 | wt. % |
| Aspartame | 0.25 | wt. % |
| Flavor | 0.25 | wt. % |

A mixture of gelatin, glycerin, oil, aspartame and flavor (up to a weight of 200 kg) are placed in a cooking tank with 800 L of capacity. The cooking tank is heated to 80-85° C. and the temperature is maintained for a period of 2-3 hours. The temperature of the cooking tank is then reduced to 55° C. until all air bubbles are completely removed. Softgel capsules of 20-oval size are then produced using conventional soft capsule machinery. The softgel capsules are filled with an A. muscaria extract.

The softgel capsules may further contain additional active agents such as proteins or amino acids, carbohydrates, lipids, vitamins, minerals and cofactors, natural or artificial flavors, dyes or other coloring additives, and preservatives. Flavors incorporated in the softgel capsules may include flavor concentrates known to those of skill, for example, flavor concentrates that replicate different food and drink flavors. Suitable oils for incorporation into a softgel capsule in an embodiment of the invention include an edible oil, extracts, and oil concentrations or combinations, blends, or mixtures thereof. Additional excipients may also be added including disintegrating agents such as cellulose and fillers such as starch.

### Example 43: Formulation of a Nutraceutical Powder

A nutraceutical powder comprising an A. muscaria extract is made as follows:

| Ingredient | Quantity (per 77 gram scoop) |
|---|---|
| A. muscaria extract | 10% wt |
| Carbohydrate (such as sweet potato flour) | 30% wt |
| Fat (such as flax seed powder) | 30% wt |
| Protein (such as egg white protein powder) | 30% wt |

A nutraceutical formulation is provided by simultaneously combining A. muscaria extract with carbohydrates, fats, and proteins in powdered format. A. muscaria extract can be used alone or in combination with other active agents, such as fatty acids, adaptogens, minerals, vitamins, amino acids, enzymes, probiotics or prebiotics. Additional excipients such as minerals, flavoring agents, non-protein mass, or free-flowing agents such as magnesium stearate may also be added. The listed carbohydrates, fats, and proteins, may be substituted with appropriate alternatives. The nutraceutical powder may be mixed with a liquid prior to consumption. In one embodiment, two scoops of 77 grams each of the nutraceutical formulation powder are mixed with 12-16 oz of cold water or any suitable liquid, including a milk such as cow's milk, almond milk, coconut milk, hemp milk, soy milk, rice milk, or cashew milk; yogurt or kefir; or a smoothie or shake.

### Example 44: Formulation of Infused Chocolate

A. muscaria extract infused chocolate is made as follows:

| Ingredient | Quantity (per 50 gram chocolate bar) | |
|---|---|---|
| A. muscaria extract | 250 | mg |
| Coconut oil | 3000 | mg |
| Chocolate | 46450 | mg |
| Lecithin | 200 | mg |
| Xanthan gum | 100 | mg |

The coconut oil is heated to between 120 and 220° F. A. muscaria extract is added, followed by the emulsifiers. The emulsifiers can be used individually or in combination. The heated mixture is blended in a high-speed blender for two minutes. The mixture is allowed to cool to room temperature. The chocolate is then melted and added, before being allowed to temper. The resultant mixture is then deposited in a mold then cooled to 55° F. Alternatively, 25 mg of A. muscaria compound(s) may be used in place of A. muscaria extract, following the same procedure outlined above.

It should be readily appreciated that the above formulation examples are illustrative only. Accordingly, any of the compounds may be substituted with the same compound in a different dosage amount. It will also be understood that reference to particular compounds and/or extracts is merely illustrative. Thus, it should be appreciated that, e.g., reference to “compound” may include one or both of muscimol and muscarine, and may additionally indicate ibotenic acid (although, in preferred embodiments, the concentration of ibotenic acid is relatively low, e.g., in relation to unextracted A. muscaria fungal material, trace, or zero); while A. muscaria extract may be an AME-1 extract or may be replaced by any other A. muscaria extract.

In some embodiments, the disclosed A. muscaria composition will also contain one or more vitamins or minerals such as potassium, vitamin D, niacin, pantothenic acid, and copper.

In some embodiments, a formulation of the invention will be prepared so as to increase an existing therapeutic effect, provide an additional therapeutic effect, increase a desired property such as stability or shelf-life, decrease an unwanted effect or property, alter a property in a desirable way (such as pharmacokinetics or pharmacodynamics), modulate a desired system or pathway (e.g., a neurotransmitter system), or provide synergistic effects.

“Synergistic effects” would be understood as including changes in potency, bioactivity, bioaccessibility, bioavailability, therapeutic effect and/or other therapeutically or pharmaceutically relevant property, that are greater than the additive contributions of the components acting alone, and/or are greater than the contribution of the isolated compounds on their own. For example, synergistic effects may include increasing an existing therapeutic effect, providing an additional therapeutic effect, increasing a desired property such as stability or shelf-life, decreasing an unwanted effect or property, altering a property in a desirable way (such as pharmacokinetics or pharmacodynamics), or modulating a desired system or pathway (e.g., a neurotransmitter system, such as the gabaminergic, glutaminergic, or acetylcholinergic systems). Numerous methods known to those of skill exist to determine whether there is synergy as to a particular effect, i.e., whether, when two or more components are mixed together, the effect is greater than the sum of the effects of the individual components when applied alone, thereby producing “1+1>2.” One such method is the isobologram analysis (or contour method) (see Huang et al. (2019). Front. Pharmacol. 10:1222).

In some embodiments the synergistic effects also may be described as “entourage-enhanced” effects, where a composition has beneficial effects in a human or other mammal, when taken for its intended use, that are greater than the beneficial effects that would be obtained or expected from each of the same compounds when taken alone (e.g., in isolated and purified form), and where those compounds occur naturally in a single organism, such as an A. muscaria fungus. For instance, in some embodiments, there are beneficial effects of muscimol and muscarine, that may be administered in combination as isolated or purified compounds, or may be administered as part of a disclosed A. muscaria extract (e.g., AME-1), including as a whole plant extract or a purified extract (including further concentrated extracts).

“Therapeutic effects” that may be increased or added In some embodiments of the invention include antioxidant, anti-inflammatory, analgesic, antineuropathic, antinociceptive, antimigraine, anxiolytic, antidepressant, antipsychotic, anti-PTSD, dissociative, immunostimulant, anti-cancer, antiemetic, orexigenic, antiulcer, antihistamine, antihypertensive, anticonvulsant, antiepileptic, bronchodilator, neuroprotective, entactogenic, empathogenic, entheogenic, euphoric, psychedelic, sedative, and stimulant effects.

The goal of increasing an existing therapeutic effect, providing an additional therapeutic effect, increasing a desired property such as stability or shelf-life, decreasing an unwanted effect or property, altering a property in a desirable way (e.g., pharmacodynamics or pharmacokinetics), modulating a desired system or pathway (e.g, a neurotransmitter system), or otherwise inducing synergy, In some embodiments, is achieved by including an additional active agent. In some embodiments, the additional active agent is any one or more of amino acids, antioxidants, anti-inflammatory agents, analgesics, antineuropathic and antinociceptive agents, antimigraine agents, anxiolytics, antidepressants, antipsychotics, anti-PTSD agents, cannabinoids, dissociatives, immunostimulants, anti-cancer agents, antiemetics, orexigenics, antiulcer agents, antihistamines, antihypertensives, anticonvulsants, antiepileptics, bronchodilators, neuroprotectants, entheogens, entactogens and empathogens, psychedelics, monoamine oxidase inhibitors, tryptamines, phenethylamines, sedatives, and stimulants. These agents may be in ion, freebase, or salt form, and may be isomers, prodrugs, or derivatives.

In some embodiments, an additional active agent is an agent useful in treating an anxiety disorder, which may include an anxiolytic drug. “Anxiolytic drug” refers to any compound or composition useful in the pharmacotherapeutic treatment of an anxiety disorder, including such compounds and compositions that modulate neurotransmission at one or more receptor systems (such as a gabaminergic, glutaminergic, cholinergic, serotonergic, adrenergic, dopaminergic, histaminergic, glycinergic, opioid, or other systems). Non-limiting examples of anxiolytic drugs include alpha blockers, antihistamines, barbiturates, beta blockers, carbamates, chlordiazepoxide, opioids, and benzodiazepines.

In some embodiments, an additional active agent is an agent useful in treating a substance use disorder, including as non-limiting examples, an opioid antagonist (e.g., nalmefene, naltrexone), a CB-1 antagonist (e.g., rimonabant), a CRH1 receptor antagonist (e.g., verucerfont, pexacerfont), a NK1R antagonist (e.g., tradipitant), an OTR agonist (e.g., oxytocin), a GABA agent (e.g., topiramate, baclofen, a benzodiazepine, such as alprazolam, diazepam or lorazepam), a voltage-gated sodium channel inhibitor (e.g., oxacarbazepine, valproic acid, zonisamide), a voltage-dependent calcium channel agonist (e.g., gabapentin, pregabalin), an α7 nicotinic acetylcholine receptor agonist (e.g., varenicline), a 5-HT3 antagonist (e.g., ondansetron), a 5-HT1A receptor partial agonist (e.g., aripiprazole), a 5-HT2A receptor antagonist (e.g., quetiapine, olanzapine, mirtazapine), a 5-HT reuptake inhibitor (e.g., trazodone), a SERT inhibitor (e.g., duloxetine), an α1 adrenoreceptor antagonist (e.g., doxazosin, prazosin), a glucocorticoid receptor antagonist (e.g., mifepristone), an α1 adrenoreceptor agonist (e.g., guanfacine), an AChE inhibitor (e.g., citicoline), a dopamine D2 receptor antagonist (e.g., tiapride), an α2 adrenoreceptor agonist (e.g., clonidine), an NMDA receptor antagonist (e.g., acamprosate), an aldehyde dehydrogenase inhibitor (e.g., disulfiram), and pharmaceutically acceptable salts thereof.

In some embodiments, an additional active agent is an agent useful in treating a sleep disorder, including a sedative-hypnotic, such as a Z-drug.

In some embodiments, an additional active agent is an agent useful in treating pain or a pain disorder, including a “pain drug,” which refers to any compound or composition useful in the pharmacotherapeutic treatment of pain or a pain disorder, including one or more analgesic drugs, usually classified into three groups: primary non-opioid, opioid, and co-analgesics, also known as adjuvants. Non-opioid analgesic drugs include acetaminophen and non-steroidal anti-inflammatory drugs or NSAIDs. These drugs are effective for mild to moderate pain, but may have significant side-effects such as liver damage in the case of acetaminophen, and gastric ulcers in the case of NSAIDs. Opioid drugs include natural substances (“opiates”) such as opium, opium-derived substances, such as morphine, and semi-synthetic and synthetic substances, such as fentanyl. Co-analgesic medications are drugs that typically address indications other than pain relief, but possess analgesic action for certain painful conditions. An example of a co-analgesic drug is gabapentin, which has a primary indication for the treatment of epilepsy, but is effective in treating certain neuropathic pain.

In some embodiments, an additional active agent useful in treating pain or a pain disorder includes any of non-peptide opioids, opioid and opioid-like peptides and their analogs, NMDA-receptor antagonists, sodium channel blockers, calcium channel blockers, adrenergic antagonists, gabaergic agonists, glycine agonists, cholinergic agonists, adrenergic agonists, epinephrine, anticonvulsants, Rho kinase inhibitors, PKC inhibitors, p38-MAP kinase inhibitors, ATP receptor blockers, endothelin receptor blockers, chemokines, interleukin and tumor necrosis factor blockers, pro-inflammatory cytokines, tricyclic antidepressants, serotonergic antagonists, serotonergic agonists, NSAIDs and COXIBs, acetaminophen, analgesic peptides, toxins, TRP channel agonists and antagonists, cannabanoids, antagonists of pro-nociceptive peptide neurotransmitter receptors CGRP1 and CGRP2, antagonists of pro-nociceptive peptide neurotransmitter receptor NK1, antagonists of pro-nociceptive peptide neurotransmitter receptor NK2, antagonists of pro-nociceptive peptide neurotransmitter receptor Y1-5, antagonists of pro-nociceptive peptide neurotransmitter receptors VPAC2, VPAC1 or PAC1, antagonists of pro-nociceptive peptide neurotransmitter receptors Gall-3 or GalR1-3, agonists or antagonists of vasopressin, corticotropin releasing hormone (CRH), growth hormone releasing hormone (GHRH), luteinizing hormone releasing hormone (LHRH), somatostatin growth hormone release inhibiting hormone, thyrotropin releasing hormone (TRH), glial-derived neurotrophic factor (GDNF), brain-derived neurotrophic factor (BDNF), nerve growth factor (NGF), neurotrophin-3 (NT-3), pancreatic polypeptide, peptide tyrosine-tyrosine, (GLP-1), peptide histidine isoleucine (PHI), pituitary adenylate cyclase activating peptide (PACAP), brain natriuretic peptide, cholecystokinin (CCK), islet amyloid polypeptide (IAPP) or amylin, melanin concentrating hormone (MCH), melanocortins (ACTH, α-MSH and others), neuropeptide FF (F8Fa), neurotensin, parathyroid hormone related protein, calcitonin, Agouti gene-related protein (AGRP), cocaine and amphetamine regulated transcript (CART)/peptide, 5-HT-moduline, hypocretins/orexins, nocistatin, prolactin releasing peptide, secretoneurin, urocortin and derivatives and analogs thereof.

In some embodiments, an additional active agent is an agent useful in treating an inflammatory disease or disorder, including NSAIDs and corticosteroids.

In some embodiments, an additional active agent useful in treating immune or autoimmune disorders includes any of corticosteroids, NSAIDs, COX-2 inhibitors, biologics, small molecule immunomodulators, non-steroidal immunophilin-dependent immunosuppressants, 5-amino salicylic acid, DMARDs, hydroxychloroquine sulfate, penicillamine, microtubule inhibitors, topoisomerase inhibitors, platins, alkylating agents, anti-metabolites, 1-D-ribofuranosyl-1,2,4-triazole-3 carboxamide, 9-[(2-hydroxyethoxy)methyl]guanine succinate, adamantanamine, 5-iodo-2′-deoxyuridine, trifluorothymidine, interferon, adenine arabinoside, protease inhibitors, thymidine kinase inhibitors, sugar or glycoprotein synthesis inhibitors, structural protein synthesis inhibitors, attachment and adsorption inhibitors, and nucleoside analogs, such as acyclovir, penciclovir, valacyclovir, and ganciclovir.

In some embodiments, an additional active agent is a serotonergic agent. In some embodiments, a “serotonergic agent” refers to any compound that binds to, blocks, or otherwise influences (e.g., via an allosteric reaction) activity at one or more serotonin receptors, including any one or more serotonin receptor subtypes. In some embodiments, a serotonergic agent binds to a serotonin receptor. In some embodiments, a serotonergic agent indirectly affects a serotonin receptor, e.g., via interactions affecting the reactivity of other molecules at the serotonin receptor. In some embodiments, a serotonergic agent is an agonist, e.g., a compound activating a serotonin receptor. In some embodiments, a serotonergic agent is an antagonist, e.g., a compound binding but not activating a serotonin receptor, e.g., blocking a receptor. In some embodiments, a serotonergic agent is an effector molecule, e.g., a compound binding to an enzyme for allosteric regulation. In some embodiments, a serotonergic agent acts (either directly or indirectly) at more than one type of receptor, including receptors other than serotonergic or other monoaminergic receptors. In some embodiments, a serotonergic agent blocks the serotonin transporter (SERT) and results in an elevation of the synaptic concentration of serotonin, and an increase of neurotransmission. In some embodiments, a serotonergic agent is a serotonin uptake or reuptake inhibitor. In some embodiments, a serotonergic agent acts as a reuptake modulator and inhibits the plasmalemmal transporter-mediated reuptake of serotonin from the synapse into the presynaptic neuron, leading to an increase in extracellular concentrations of serotonin and an increase in neurotransmission. In some embodiments, a serotonergic agent inhibits the activity of one or both monoamine oxidase enzymes, resulting in an increase in concentrations of serotonin and an increase in neurotransmission. In some embodiments, a serotonergic agent is an antidepressant or anxiolytic, such as an SSRI, serotonin-norepinephrine reuptake inhibitor (SNRI), tricyclic antidepressant (TCA), monoamine oxidase inhibitor (MAOI), or atypical antidepressant. In other embodiments, a serotonergic agent is selected from the group consisting of: (1) serotonin transport inhibitors; (2) serotonin receptor modulators; (3) serotonin reuptake inhibitors; (4) serotonin and norepinephrine reuptake inhibitors; (5) serotonin dopamine antagonists; (6) monoamine reuptake inhibitors; (7) pyridazinone aldose reductase inhibitors; (8) stimulants of serotonin receptors; (9) stimulants of serotonin synthesis; (10) serotonin receptor agonists; (11) serotonin receptor antagonists; and (12) serotonin metabolites.

For any of the compounds and additional active agents, substitution of the compound or active agent by its ion, free base, salt form, polymorph, solvate form, or an isomer or enantiomerically enriched mixture, shall be understood to provide merely an alternative embodiment still within the scope of the invention (with modifications to the formulation and dosage amounts made according to the teachings herein and ordinary skill, if necessary or desired). Further, compositions within the scope of the invention should be understood to be open-ended and may include additional active or inactive agents and ingredients.

The type of formulation employed for the administration of the compounds and/or extracts employed in the methods of the invention generally may be dictated by the compounds and/or extracts employed, the type of pharmacokinetic profile desired from the route of administration of the compounds and/or extracts, and the state of the patient. It will be readily appreciated that any of the above embodiments and classes of embodiments can be combined to form additional embodiments and formulations.

### F. ROUTE OF ADMINISTRATION

The disclosed pharmaceutical compositions are suitable for administration by a variety of routes. Non-limiting examples of routes of administration include enteral administration, such as oral, sublingual, buccal, and rectal administration; parenteral administration, including bolus injection or continuous infusion, intravenous, intra-arterial, intraperitoneal, intraosseous, intramuscular, intrathecal, intracerebroventricular, vaginal, ocular, nasal, cutaneous, topical, otic, ocular, transdermal, and subcutaneous administration.

In some embodiments, a pharmaceutical composition is administered as oral solid and oral liquid dosage forms; sublingually or buccally; as injections, including intravenous, intra-arterial, intraperitoneal, intraosseous, intramuscular, intrathecal, and intracerebroventricular; rectally, vaginally, ocularly, nasally, cutaneously, topically, oticly, transdermally, and subcutaneously.

In some embodiments, in which administration is enteral, parenteral, or both, an effective amount of the compounds and/or extracts described herein are systemically administered to a subject. In some embodiments, an effective amount of the compounds and/or extracts described herein is administered orally to a subject. In some embodiments, an effective amount of the compounds and/or extracts described herein is intravenously administered to a subject. In some embodiments, an effective amount of the compounds and/or extracts described herein is administered by inhalation to a subject. In some embodiments, an effective amount of the compounds and/or extracts described herein is administered by nasal administration to a subject. In some embodiments, an effective amount of the compounds and/or extracts described herein is administered by injection to a subject. In some embodiments, an effective amount of the compounds and/or extracts described herein is administered topically (dermal) to a subject. In some embodiments, an effective amount of the compounds and/or extracts described herein is administered by ophthalmic administration to a subject. In some embodiments, an effective amount of the compounds and/or extracts described herein is administered rectally to a subject. In some embodiments, the compounds and/or extracts described herein and employed in the methods described herein are effectively administered to a subject via other means, and prepared as any acceptable composition known to those of skill. In some embodiments, such compositions may be prepared in any manner known in the pharmaceutical arts that comprise at least one bioactive molecule (Sheth et al., 1980).

In some embodiments, the compounds and/or extracts described herein are administered by multiple routes, which may differ between subjects, such as a patient, according to subject preferences, comorbidities, side effect profiles, pharmacokinetic and pharmacodynamic considerations, and other factors. In some embodiments are the presence of other substances with the compounds and/or extracts known to those skilled in the art, such as modifications in the preparation to facilitate absorption through various routes (e.g., gastrointestinal, transdermal, etc.), to extend the effect of the drugs, and/or attain higher or more stable serum levels or enhance the therapeutic effect of the compounds and/or extracts described herein.

In some embodiments, the pharmaceutical compositions are suitable as oral solid or oral liquid dosage forms, administered sublingually, buccally, topically, rectally, vaginally, ocularly, oticly, nasally, cutaneously, topically, and transdermally; or as intravenous, intra-arterial, intraperitoneal, intraosseous, intramuscular, intrathecal, intracerebroventricular, and subcutaneous injection, wherein such injections comprise physiologically acceptable sterile aqueous or non-aqueous solutions, dispersions, suspensions or emulsions, liposomes, and sterile powders for reconstitution into sterile injectable solutions or dispersions.

In some embodiments, a pharmaceutical composition may be administered via enteral or parenteral means, wherein enteral means includes, but is not limited to, oral solid and oral liquid dosage forms, sublingual and buccal administration, and rectal administration; and parenteral administration means includes, but is not limited to, bolus injection or continuous infusion, intravenous, intra-arterial, intraperitoneal, intraosseous, intramuscular, intrathecal, intracerebroventricular, vaginal, ocular, nasal, cutaneous, topical, otic, transdermal, and subcutaneous administration; in addition to other equivalent means known to those of skill.

Enteral administration includes administration involving any part of the gastrointestinal tract. Non-limiting examples include those by mouth, including oral solid and oral liquid dosage forms, and rectal, and in embodiments may be preferably formulated as tinctures. Parenteral administration refers to administration from any means not involving the gastrointestinal tract, including intravenous, intra-arterial, intraosseous infusion, intramuscular, intracerebral, intracerebroventricular, intrathecal, otic, ocular, vaginal, and subcutaneous. In some embodiments, parenteral administration may include sublingual and/or buccal administration.

In some embodiments, a pharmaceutical composition may be administered to a subject via a combination of administration means. In some embodiments, the pharmaceutical composition may be administered to a subject via one or more enteral administration means. In some embodiments, the pharmaceutical composition may be administered to a subject via one or more parenteral administration means. In some embodiments, the pharmaceutical composition may be administered to a subject via at least one enteral administration means, and at least one parenteral administration means. In some embodiments, an equivalent route of administration known to one of skill is utilized.

### G. METHODS OF ADMINISTRATION

In some aspects, provided are methods of administration or methods of administering a compound and/or extract disclosed herein. In one aspect are disclosed methods for using therapeutically effective amounts of the disclosed compositions comprising the disclosed compounds and/or extracts in a mammal, and preferably in a human. The mammal may be a subject or a patient. As used herein, the terms “subject,” “user,” “patient,” and “individual” are used interchangeably, and refer to any mammal, preferably a human. Such terms will be understood to include one who has an indication for which the compositions or methods described herein may be efficacious, or who otherwise may benefit by the invention. In general, all of the compositions and methods of the invention will be appreciated to work for all individuals, although individual variation is to be expected, and will be understood.

Administration of pharmaceutical compositions in an “effective amount,” a “therapeutically effective amount,” a “therapeutically effective dose,” or a “pharmacologically effective amount,” refers to an amount of a compound and/or extract that is sufficient to provide the desired therapeutic effect, for example, relieving to some extent one or more of the symptoms of the disease or condition being treated. The result can be reduction and/or alleviation of the signs, symptoms, or causes of a disease or disorder, or any other desired alteration of a biological system. The term “therapeutically effective amount” includes, for example, a prophylactically effective amount. An “effective amount” of a compound and/or extract described herein is an amount effective to achieve a desired pharmacologic effect or meaningful therapeutic improvement. It is understood that “an effective amount” or “a therapeutically effective amount” can vary from subject to subject due to variation in metabolism of a compound, such as the compounds and/or extracts described herein, of age, weight, general condition of the subject, the condition being treated, the severity of the condition being treated, and the judgment of the prescribing physician. Additionally, pharmacogenomic (the effect of genotype on the pharmacokinetic, pharmacodynamic or efficacy profile of a therapeutic) information about a particular patient may affect dosage used.

“Therapeutically effective dose” refers to the dose necessary to elicit a desired result within a patient undergoing treatment. A therapeutically effective dose therefore may, In some embodiments, refer to a dose of a disclosed A. muscaria composition, e.g., an extract, compound, or analog thereof, necessary to deliver measurable patient-specific biologic effects in the treatment or prevention of a condition or disorder. A “therapeutically effective dose” may be used interchangeably with a “therapeutically effective amount” or an “effective amount.”

### H. DOSAGE

It will be readily appreciated that dosages may vary depending upon whether the treatment is therapeutic or prophylactic, the onset, progression, severity, frequency, duration, probability of or susceptibility of the symptom to which treatment is directed, clinical endpoint desired, previous, simultaneous or subsequent treatments, general health, age, gender, and race of the subject, bioavailability, potential adverse systemic, regional or local side effects, the presence of other disorders or diseases in the subject, and other factors that will be appreciated by the skilled artisan (e.g., medical or familial history).

In some embodiments, where a pharmaceutical composition includes muscimol and/or a muscimol analog, it may be present in an amount so that a single dose is (whether or not such dose is present in a unit dosage form), e.g., 2.5 mg or less (including a dose of 1.0 mg or less, 0.5 mg or less, 0.1 mg or less, and 0.05 mg or less), at least 2.5 mg, at least 3.0 mg, at least 3.5 mg, at least 4.0 mg, at least 4.5 mg, at least 5.0 mg, at least 5.5 mg, at least 6.0 mg, at least 6.5 mg, at least 7.0 mg, at least 7.5 mg, at least 8.0 mg, at least 8.5 mg, at least 9.0 mg, at least 9.5 mg, at least 10.0 mg, at least 10.5 mg, at least 11.0 mg, at least 11.5 mg, at least 12.0 mg, at least 12.5 mg, at least 13.0 mg, at least 13.5 mg, at least 14.0 mg, at least 14.5 mg, at least 15.0 mg, at least 15.5 mg, at least 16.0 mg, at least 16.5 mg, at least 17.0 mg, at least 17.5 mg, at least 18.0 mg, at least 18.5 mg, at least 19.0 mg, at least 19.5 mg, at least 20.0 mg, at least 22.5 mg, or at least 25.0 mg, as well as amounts within these ranges.

In some embodiments, where a pharmaceutical composition includes muscimol and/or a muscimol analog, it may be present in an amount so that a single dose is (in a milligram dosage amount calculated based on the kilogram weight of the patient), e.g., 0.025 mg/kg or less (including a dose of 0.010 mg/kg or less, 0.005 mg/kg or less, 0.001 mg/kg or less, and 0.0005 mg/kg or less), at least 0.050 mg/kg, at least 0.055 mg/kg, at least 0.060 mg/kg, at least 0.065 mg/kg, at least 0.070 mg/kg, at least 0.075 mg/kg, at least 0.080 mg/kg, at least 0.085 mg/kg, at least 0.090 mg/kg, at least 0.095 mg/kg, at least 0.10 mg/kg, at least 0.11 mg/kg, at least 0.12 mg/kg, at least 0.13 mg/kg, or at least 0.14 mg/kg, at least 0.15 mg/kg, at least 0.16 mg/kg, at least 0.17 mg/kg, at least 0.18 mg/kg, at least 0.19 mg/kg, at least 0.20 mg/kg, at least 0.21 mg/kg, at least 0.22 mg/kg, at least 0.23 mg/kg, at least 0.24 mg/kg, at least 0.25 mg/kg, at least 0.26 mg/kg, at least 0.27 mg/kg, at least 0.28 mg/kg, at least 0.29 mg/kg, or at least 0.30 mg/kg, as well as amounts within these ranges.

In some embodiments, where a pharmaceutical composition includes muscarine and/or a muscarine analog, it may be present in an amount so that a single dose is (whether or not such dose is present in a unit dosage form), e.g., 2.5 mg or less (including a dose of 1.0 mg or less, 0.5 mg or less, 0.1 mg or less, and 0.05 mg or less), at least 2.5 mg, at least 3.0 mg, at least 3.5 mg, at least 4.0 mg, at least 4.5 mg, at least 5.0 mg, at least 5.5 mg, at least 6.0 mg, at least 6.5 mg, at least 7.0 mg, at least 7.5 mg, at least 8.0 mg, at least 8.5 mg, at least 9.0 mg, at least 9.5 mg, at least 10.0 mg, at least 10.5 mg, at least 11.0 mg, at least 11.5 mg, at least 12.0 mg, at least 12.5 mg, at least 13.0 mg, at least 13.5 mg, at least 14.0 mg, at least 14.5 mg, at least 15.0 mg, at least 15.5 mg, at least 16.0 mg, at least 16.5 mg, at least 17.0 mg, at least 17.5 mg, at least 18.0 mg, at least 18.5 mg, at least 19.0 mg, at least 19.5 mg, at least 20.0 mg, at least 22.5 mg, or at least 25.0 mg, as well as amounts within these ranges.

In some embodiments, where a pharmaceutical composition includes muscarine and/or a muscarine analog, it may be present in an amount so that a single dose is (in a milligram dosage amount calculated based on the kilogram weight of the patient), e.g., 0.025 mg/kg or less (including a dose of 0.010 mg/kg or less, 0.005 mg/kg or less, 0.001 mg/kg or less, and 0.0005 mg/kg or less), at least 0.050 mg/kg, at least 0.055 mg/kg, at least 0.060 mg/kg, at least 0.065 mg/kg, at least 0.070 mg/kg, at least 0.075 mg/kg, at least 0.080 mg/kg, at least 0.085 mg/kg, at least 0.090 mg/kg, at least 0.095 mg/kg, at least 0.10 mg/kg, at least 0.11 mg/kg, at least 0.12 mg/kg, at least 0.13 mg/kg, or at least 0.14 mg/kg, at least 0.15 mg/kg, at least 0.16 mg/kg, at least 0.17 mg/kg, at least 0.18 mg/kg, at least 0.19 mg/kg, at least 0.20 mg/kg, at least 0.21 mg/kg, at least 0.22 mg/kg, at least 0.23 mg/kg, at least 0.24 mg/kg, at least 0.25 mg/kg, at least 0.26 mg/kg, at least 0.27 mg/kg, at least 0.28 mg/kg, at least 0.29 mg/kg, or at least 0.30 mg/kg, as well as amounts within these ranges.

In some embodiments, a pharmaceutical composition may include a mixture of A. muscaria compounds in a fixed ratio, and will contain a ratio of muscimol to muscarine (as muscimol:muscarine), including an analog of one or both, of less than 1:1, 1:1, at least 1:1, at least 2:1, at least 3:1, at least 4:1, at least 5:1, at least 6:1, at least 7:1, at least 8:1, at least 9:1, at least 10:1, at least 11:1, at least 12:1, at least 13:1, at least 14:1, at least 15:1, at least 16:1, at least 17:1, at least 18:1, at least 19:1, at least 20:1, at least 25:1, at least 30:1, at least 40:1, at least 50:1, at least 60:1, at least 70:1, at least 80:1, at least 90:1, and at least 100:1, including the exact above-listed ratios themselves. In other embodiments, these same ratios will be the ratios of muscarine to muscimol (as muscarine:muscimol), including an analog of one or both.

In some embodiments, where a pharmaceutical composition includes an A. muscaria extract comprising muscimol, it may be present in an amount so that a single dose of muscimol is (whether or not such dose is present in a unit dosage form), e.g., 2.5 mg or less (including a dose of 1.0 mg or less, 0.5 mg or less, 0.1 mg or less, and 0.05 mg or less), at least 2.5 mg, at least 3.0 mg, at least 3.5 mg, at least 4.0 mg, at least 4.5 mg, at least 5.0 mg, at least 5.5 mg, at least 6.0 mg, at least 6.5 mg, at least 7.0 mg, at least 7.5 mg, at least 8.0 mg, at least 8.5 mg, at least 9.0 mg, at least 9.5 mg, at least 10.0 mg, at least 10.5 mg, at least 11.0 mg, at least 11.5 mg, at least 12.0 mg, at least 12.5 mg, at least 13.0 mg, at least 13.5 mg, at least 14.0 mg, at least 14.5 mg, at least 15.0 mg, at least 15.5 mg, at least 16.0 mg, at least 16.5 mg, at least 17.0 mg, at least 17.5 mg, at least 18.0 mg, at least 18.5 mg, at least 19.0 mg, at least 19.5 mg, at least 20.0 mg, at least 22.5 mg, or at least 25.0 mg, as well as amounts within these ranges.

In some embodiments, where a pharmaceutical composition includes an A. muscaria extract comprising muscimol, it may be present in an amount so that a single dose of muscimol is (in a milligram dosage amount calculated based on the kilogram weight of the patient), e.g., 0.025 mg/kg or less (including a dose of 0.010 mg/kg or less, 0.005 mg/kg or less, 0.001 mg/kg or less, and 0.0005 mg/kg or less), at least 0.050 mg/kg, at least 0.055 mg/kg, at least 0.060 mg/kg, at least 0.065 mg/kg, at least 0.070 mg/kg, at least 0.075 mg/kg, at least 0.080 mg/kg, at least 0.085 mg/kg, at least 0.090 mg/kg, at least 0.095 mg/kg, at least 0.10 mg/kg, at least 0.11 mg/kg, at least 0.12 mg/kg, at least 0.13 mg/kg, or at least 0.14 mg/kg, at least 0.15 mg/kg, at least 0.16 mg/kg, at least 0.17 mg/kg, at least 0.18 mg/kg, at least 0.19 mg/kg, at least 0.20 mg/kg, at least 0.21 mg/kg, at least 0.22 mg/kg, at least 0.23 mg/kg, at least 0.24 mg/kg, at least 0.25 mg/kg, at least 0.26 mg/kg, at least 0.27 mg/kg, at least 0.28 mg/kg, at least 0.29 mg/kg, or at least 0.30 mg/kg, as well as amounts within these ranges.

In some embodiments, where a pharmaceutical composition includes an A. muscaria extract comprising muscarine, it may be present in an amount so that a single dose of muscarine is (whether or not such dose is present in a unit dosage form), e.g., 2.5 mg or less (including a dose of 1.0 mg or less, 0.5 mg or less, 0.1 mg or less, and 0.05 mg or less), at least 2.5 mg, at least 3.0 mg, at least 3.5 mg, at least 4.0 mg, at least 4.5 mg, at least 5.0 mg, at least 5.5 mg, at least 6.0 mg, at least 6.5 mg, at least 7.0 mg, at least 7.5 mg, at least 8.0 mg, at least 8.5 mg, at least 9.0 mg, at least 9.5 mg, at least 10.0 mg, at least 10.5 mg, at least 11.0 mg, at least 11.5 mg, at least 12.0 mg, at least 12.5 mg, at least 13.0 mg, at least 13.5 mg, at least 14.0 mg, at least 14.5 mg, at least 15.0 mg, at least 15.5 mg, at least 16.0 mg, at least 16.5 mg, at least 17.0 mg, at least 17.5 mg, at least 18.0 mg, at least 18.5 mg, at least 19.0 mg, at least 19.5 mg, at least 20.0 mg, at least 22.5 mg, or at least 25.0 mg, as well as amounts within these ranges.

In some embodiments, where a pharmaceutical composition includes an A. muscaria extract comprising muscarine, it may be present in an amount so that a single dose of muscarine is (in a milligram dosage amount calculated based on the kilogram weight of the patient), e.g., 0.025 mg/kg or less (including a dose of 0.010 mg/kg or less, 0.005 mg/kg or less, 0.001 mg/kg or less, and 0.0005 mg/kg or less), at least 0.050 mg/kg, at least 0.055 mg/kg, at least 0.060 mg/kg, at least 0.065 mg/kg, at least 0.070 mg/kg, at least 0.075 mg/kg, at least 0.080 mg/kg, at least 0.085 mg/kg, at least 0.090 mg/kg, at least 0.095 mg/kg, at least 0.10 mg/kg, at least 0.11 mg/kg, at least 0.12 mg/kg, at least 0.13 mg/kg, or at least 0.14 mg/kg, at least 0.15 mg/kg, at least 0.16 mg/kg, at least 0.17 mg/kg, at least 0.18 mg/kg, at least 0.19 mg/kg, at least 0.20 mg/kg, at least 0.21 mg/kg, at least 0.22 mg/kg, at least 0.23 mg/kg, at least 0.24 mg/kg, at least 0.25 mg/kg, at least 0.26 mg/kg, at least 0.27 mg/kg, at least 0.28 mg/kg, at least 0.29 mg/kg, or at least 0.30 mg/kg, as well as amounts within these ranges.

In some embodiments, where a pharmaceutical composition includes an A. muscaria extract, for instance the disclosed A. muscaria extract AME-1, it may be present in an amount so a single dose is (in a mg amount calculated based on the kg weight of the patient, and calculated based on the weight of the total A. muscaria extract, not any individual compound or component thereof), e.g., 2.5 mg/kg or less (including a dose of 1.0 mg/kg or less, 0.5 mg/kg or less, 0.1 mg/kg or less, and 0.05 mg/kg or less), at least 5.0 mg/kg, at least 5.5 mg/kg, at least 6.0 mg/kg, at least 6.5 mg/kg, at least 7.0 mg/kg, at least 7.5 mg/kg, at least 8.0 mg/kg, at least 8.5 mg/kg, at least 9.0 mg/kg, at least 9.5 mg/kg, at least 10 mg/kg, at least 11 mg/kg, at least 12 mg/kg, at least 13 mg/kg, or at least 14 mg/kg, at least 15 mg/kg, at least 16 mg/kg, at least 17 mg/kg, at least 18 mg/kg, at least 19 mg/kg, at least 20 mg/kg, at least 21 mg/kg, at least 22 mg/kg, at least 23 mg/kg, at least 24 mg/kg, at least 25 mg/kg, at least 26 mg/kg, at least 27 mg/kg, at least 28 mg/kg, at least 29 mg/kg, or at least 30 mg/kg, as well as amounts within these ranges.

In some embodiments, where a pharmaceutical composition includes an A. muscaria extract, for instance the disclosed A. muscaria extract AME-1, it may be present in an amount so that a single dose is (whether or not such dose is present in a unit dosage form, and calculated based on the weight of the total A. muscaria extract, not any individual compound or component thereof), e.g., 250 mg or less (including a dose of 100 mg or less, 50 mg or less, 10 mg or less, and 5 mg or less), at least 250 mg, at least 300 mg, at least 350 mg, at least 400 mg, at least 450 mg, at least 500 mg, at least 550 mg, at least 600 mg, at least 650 mg, at least 700 mg, at least 750 mg, at least 800 mg, at least 850 mg, at least 900 mg, at least 950 mg, at least 1,000 mg, at least 1,050 mg, at least 1,100 mg, at least 1,150 mg, at least 1,200 mg, at least 1,250 mg, at least 1,300 mg, at least 1,350 mg, at least 1,400 mg, at least 1,450 mg, at least 1,500 mg, at least 1,550 mg, at least 1,600 mg, at least 1,650 mg, at least 1,700 mg, at least 1,750 mg, at least 1,800 mg, at least 1,850 mg, at least 1,900 mg, at least 1,950 mg, at least 2,000 mg, at least 2,250 mg, or at least 2,500 mg, as well as amounts within these ranges.

In some embodiments, where a pharmaceutical composition includes an A. muscaria extract, for instance the disclosed A. muscaria extract AME-1, it may be present in an amount so a single dose is (whether or not the dose is present in a unit dosage form, and calculated based on the volume of the total A. muscaria extract, not any individual compound or component thereof), e.g., 25 mL or less (including a dose of 10 mL or less, 5 mL or less, 1 mL or less, and 0.5 mL or less), at least 25 mL, at least 30 mL, at least 35 mL, at least 40 mL, at least 45 mL, at least 50 mL, at least 55 mL, at least 60 mL, at least 65 mL, at least 70 mL, at least 75 mL, at least 80 mL, at least 85 mL, at least 90 mL, at least 95 mL, at least 100 mL, at least 105 mL, at least 110 mL, at least 115 mL, at least 120 mL, at least 125 mL, at least 130 mL, at least 135 mL, at least 140 mL, at least 145 mL, at least 150 mL, at least 155 mL, at least 160 mL, at least 165 mL, at least 170 mL, at least 175 mL, at least 180 mL, at least 185 mL, at least 190 mL, at least 195 mL, at least 200 mL, at least 225 mL, or at least 250 mL, as well as amounts within these ranges.

In some embodiments, where a pharmaceutical composition includes an additional active agent, for instance an additional active agent as described herein, it may be present in an amount so that a single dose is (in a milligram dosage amount calculated based on the kilogram weight of the patient), e.g., 0.25 mg/kg or less (including a dose of 0.10 mg/kg or less, 0.05 mg/kg or less, 0.01 mg/kg or less, and 0.005 mg/kg or less), at least 0.50 mg/kg, at least 0.55 mg/kg, at least 0.60 mg/kg, at least 0.65 mg/kg, at least 0.70 mg/kg, at least 0.75 mg/kg, at least 0.80 mg/kg, at least 0.85 mg/kg, at least 0.90 mg/kg, at least 0.95 mg/kg, at least 1.0 mg/kg, at least 1.1 mg/kg, at least 1.2 mg/kg, at least 1.3 mg/kg, or at least 1.4 mg/kg, at least 1.5 mg/kg, at least 1.6 mg/kg, at least 1.7 mg/kg, at least 1.8 mg/kg, at least 1.9 mg/kg, at least 2.0 mg/kg, at least 2.1 mg/kg, at least 2.2 mg/kg, at least 2.3 mg/kg, at least 2.4 mg/kg, at least 2.5 mg/kg, at least 2.6 mg/kg, at least 2.7 mg/kg, at least 2.8 mg/kg, at least 2.9 mg/kg, or at least 3.0 mg/kg, as well as amounts within these ranges.

In some embodiments, where a pharmaceutical composition includes an additional active agent, for instance an additional active agent as described herein, it may be present in an amount so that a single dose is (whether or not such dose is present in a unit dosage form), e.g., 25 mg or less (including a dose of 10 mg or less, 5 mg or less, 1 mg or less, and 0.5 mg or less), at least 25 mg, at least 30 mg, at least 35 mg, at least 40 mg, at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185 mg, at least 190 mg, at least 195 mg, at least 200 mg, at least 225 mg, or at least 250 mg, as well as amounts within these ranges.

It will be understood that, In some embodiments, the dose actually administered will be determined by a physician, in light of the relevant circumstances, the method of delivery and route of administration, the age of the patient, the weight of the patient, whether the patient has any comorbidities, other medications the patient is taking (routinely or presently), and any patient-specific aspects that could affect the way in which the compounds and/or extracts interact with the patient, such as variations in metabolism, variations in patient response, etc., and therefore any dosage ranges disclosed herein are not intended to limit the scope of the invention. In some instances, dosage levels below the lower limit of a disclosed range may be more than adequate, while in other cases doses above a range may be employed without causing any harmful side effects, provided for instance that such larger doses also may be divided into several smaller doses for administration, either taken together or separately.

In some embodiments, the compounds and/or extracts may be administered and dosed in accordance with good medical practice, taking into account the method and scheduling of administration, prior and concomitant medications and medical supplements, the clinical condition of the individual patient and the severity of the underlying disease, the patient's age, sex, body weight, and other such factors relevant to medical practitioners, and knowledge of the particular compound(s) used. Dosage levels may differ from patient to patient, for individual patients across time, and for different formulations, but shall be able to be determined with ordinary skill. Determination of appropriate dosing shall include not only the determination of single dosage amounts, but also the determination of the number and timing of doses. Dose amount, frequency or duration may be increased or reduced, as indicated by the clinical outcome desired, status of the pathology or symptom, any adverse side effects of the treatment or therapy, or concomitant medications. A skilled artisan with the teachings of this disclosure in hand will appreciate the factors that may influence the dosage, frequency, and timing required to provide an amount sufficient or effective for providing a therapeutic effect or benefit, and to do so depending on the type of effect or benefit desired, as well as to avoid or reduce adverse effects.

In some instances, certain personalized approaches (i.e., “personalized” or “precision” medicine) may be utilized, based on individual characteristics, including drug metabolism (e.g., CYP3A4) or individual genetic variation. The term “genetic variation” refers to a change in a gene sequence relative to a reference sequence (e.g., a commonly-found and/or wild-type sequence). Genetic variation may be recombination events or mutations such as substitution/deletion/insertion events like point and splice site mutations. In an embodiment, the genetic variation is a genetic variation in one or more cytochrome P450 enzymes that affect drug metabolism, including metabolism of a disclosed compound or composition, such as any one or more of the enzymes CYP1A1, CYP1A2, CYP1B1, CYP2A6, CYP2A13, CYP2B6, CYP2C8, CYP2C9, CYP2C18, CYP2D6, CYP2C19, CYP2E1, CYP2G1, CYP2J2, CYP2R1, CYP2S1, CYP3A4, CYP3A5, CYP3A5P1, CYP3A5P2, CYP3A7, CYP4A11, CYP4B1, CYP4F2, CYP4F3, CYP4F8, CYP4F11, CYP4F12, CYP4X1, CYP4Z1, CYP5A1, CYP7A1, CYP7B1, CYP8A1, CYP8B1, CYP11A1, CYP11B1, CYP11B2, CYP17, CYP19, CYP21, CYP24, CYP26A1, CYP26B1, CYP27A1, CYP27B1, CYP39, CYP46, and CYP51.

In some embodiments, a disclosed composition is taken together with a compound that is metabolized by the same CYP enzyme(s) as a disclosed compound or composition, so as to permit a lower dose to be taken, increase the effective bioavailability of one or both, or otherwise affect drug metabolism or pharmacokinetics. In some embodiments, the dose of a disclosed composition is adjusted when administered to a subject known to be a “poor metabolizer” of the compounds and/or extracts in the composition.

In other embodiments, appropriate dosages to achieve a therapeutic effect, including the upper and lower bounds of any dose ranges, can be determined by an individual, including an individual who is not a clinician, by reference to available public information and knowledge, and reference to subjective considerations regarding desired outcomes and effects.

### I. METHODS OF USE

In some aspects, provided herein are methods of using a disclosed A. muscaria composition, e.g., an A. muscaria extract or an A. muscaria compound, including an A. muscaria analog (together for shorthand, “A. muscaria composition”) to improve health and wellness. In some embodiments, disclosed is use of an A. muscaria composition to improve health and wellness. In some embodiments, disclosed is use of A. muscaria compositions in the manufacture of a medicament to improve health and wellness. In some embodiments, administering the disclosed A. muscaria compositions to a subject improve health and wellness in the subject.

a. Exemplary Use to Modulate Neurotransmission

In some aspects, provided are methods of using the disclosed A. muscaria compositions to modulate neurotransmission. In some embodiments, the disclosed A. muscaria compositions are used in the manufacture of a medicament to modulate neurotransmission. In some embodiments, the disclosed A. muscaria compositions are administered, e.g., in a therapeutically effective amount, to a subject to modulate neurotransmission in said subject. In some embodiments, modulating neurotransmission promotes health and wellness. In some embodiments, modulating neurotransmission results in an improvement, such as clinical improvement, of a condition, such as a disease or a disorder.

In some embodiments, the disclosed A. muscaria compositions modulate neurotransmission. In some embodiments, modulating neurotransmission comprises regulating levels of neurotransmitters, e.g., amino acid neurotransmitters, in, for example, the CNS and peripheral tissues. In some embodiments, modulating neurotransmission comprises increasing levels of neurotransmitters, e.g., amino acid neurotransmitters, in, for example, the CNS and peripheral tissues of a subject to whom a disclosed A. muscaria composition, e.g., an extract, compound, or analog thereof has been administered. In some embodiments, modulating neurotransmission comprises decreasing levels of neurotransmitters, e.g., amino acid neurotransmitters, in, for example, the CNS and peripheral tissues of a subject to whom a disclosed A. muscaria composition, e.g., an extract, compound, or analog thereof has been administered. In some embodiments, modulating neurotransmission by administering a disclosed A. muscaria composition, e.g., extracts, compounds, or analog thereof to a subject treats a condition, such as a disease or disorder in the subject.

In some embodiments, the disclosed A. muscaria compositions modulate gabaminergic neurotransmission. In some embodiments, the disclosed A. muscaria compositions modulate glutaminergic neurotransmission. In some embodiments, the disclosed A. muscaria compositions modulate cholinergic neurotransmission. In some embodiments, the disclosed A. muscaria compositions modulate any one or more of gabaminergic, glutaminergic, and cholinergic neurotransmission.

Muscimol (3 mg/kg) and ibotenic acid (16 mg/kg), administered to male rats and mice intraperitoneally (n=3-11) has been shown to affect the levels of serotonin (5-hydroxytryptamine), noradrenaline and dopamine in the brain, akin to LSD (10 mg/kg) (Konig-Bersin et al., Psychopharmacologia 1970; 18:1-10). However, in terms of mechanisms, neither ibotenic acid nor muscimol appears to act on acetylcholine, dopamine, or 5-hydroxytryptamine receptors in the central nervous system. Instead both muscimol and ibotenic acid were shown to act similarly by activation of the GABA receptor (Brehm et al., Acta Chim Scand 1972; 26:1298-1299; Walker et al., Comp Gen Pharmacol 1971; 2:168-174). Worms et al., confirmed that muscimol is a GABA agonist of high affinity but of relatively low efficacy (Worms et al., Life Sciences 1979; 25:607-614).

GABA_{A} receptors are ion channels which can be activated by the neurotransmitter GABA, or by drugs (e.g., muscimol). When these channels open they are permeable to negatively charged chloride ions (Cl⁻). As a result, muscimol can either inhibit or stimulate nerves, depending on the relative concentration of Cl⁻ inside and outside the cell. Accordingly, GABA_{A} receptors are extremely important for regulating nerve activity throughout the nervous system. When dysregulated, an individual often experiences anxiety. However, as GABA_{A} regulates nerve activity, it also plays a role in pain stimulation. Joint pain, for instance, is triggered by nociceptors. Nociceptors express GABA_{A} receptors. Activation of these receptors leads to depolarization of nociceptors and ultimately inhibition of the transfer of pain information into the spinal cord. In some embodiments, by selectively activating nociceptor GABA_{A} receptors, rather than those in the brain, provides a localized antinociceptive (pain-killing) effect on the joint. In some embodiments, a cream comprising bioactive compounds from A. muscaria that is administered locally to an arthritic joint, as in the compositions and methods disclosed herein, has a beneficial effect on joint pain.

Most depressant and sedative drugs such as benzodiazepine tranquilizers, barbiturates, anesthetics, and alcohol are believed have a modulatory effect on the GABA_{A} receptor by binding to allosteric sites on GABA_{A} receptors where they enhance the actions of GABA in accumulating negatively charged chloride ions into the cell, inducing sedative or anesthetic effects. Long-term administration of benzodiazepines results in the development of tolerance to some of the effects, reducing their clinical efficacy. In some embodiments, the disclosed A. muscaria compositions offer significant benefits versus other gabaminergic compounds like benzodiazepines and barbiturates. In some embodiments, the disclosed A. muscaria compositions comprise a selective GABA_{A} agonist that binds to and directly activates GABA_{A} receptors.

Muscarine mimics the action of the neurotransmitter acetylcholine by agonizing muscarinic acetylcholine receptors, which modulates cholinergic neurotransmission. There are five different types of muscarinic receptors: M1, M2, M3, M4 and M5. The M2 and M3 subtypes mediate muscarinic responses at peripheral autonomic tissues; M1 and M4 subtypes are more abundant in brain and autonomic ganglia. M1, M3, and M5 interact with Gq proteins to stimulate phosphoinositide hydrolysis and the release of intracellular calcium. M2 and M4, by contrast, interact with Gi proteins to inhibit adenylyl cyclase, which results in a decrease of intracellular concentration of cyclic adenosine monophosphate (cAMP). Muscarinic receptors also signal via other pathways, for instance via G beta-gamma complex modulation of potassium channels. This allows muscarine to modulate cellular excitability via the membrane potential.

Detecting a change in neurotransmitter levels in a subject, such as an increase or a decrease, can be achieved according to methods known to one of skill, for example, brain microdialysis (Chefer et al., Curr Protoc Neurosci. 2009; Chapter: Unit 7.1; Darvesh et al., Expert Opin Drug Discov. 2011; 6(2): 109-127) and brain imaging, for example, positron emission tomography (PET) and single photon emission computed tomography (SPECT) (see e.g., Wong & Gjedde, Encyclopedia of Neuroscience, 2009; 939-952 and Takano, Front Psychiatry., 2018; 9:228).

b. Exemplary Nutraceutical Use

In some aspects, the disclosed A. muscaria compositions are used as nutraceuticals, e.g., dietary supplements. In some embodiments, the disclosed A. muscaria compositions are used in the manufacture of a nutraceutical, e.g., a dietary supplement. In some embodiments, the disclosed A. muscaria compositions are administered to a subject as a nutraceutical, e.g., a dietary supplement. In some embodiments, the disclosed A. muscaria compositions are used as a dietary ingredient in nutraceuticals, e.g., dietary supplements.

Although sections herein and associated disclosure are separated into “nutraceutical use” and “pharmaceutical use” it will be readily appreciated that such uses are not mutually exclusive, and these sections are merely provided to help illustrate certain exemplary embodiments.

In some embodiments, disclosed A. muscaria compositions may promote sensations of calmness. In some embodiments, disclosed A. muscaria compositions may induce a trance-like state. In some embodiments, disclosed A. muscaria compositions may benefit mood, increasing enjoyment and giving rise to a feeling of euphoria. In some embodiments, disclosed A. muscaria compositions may relieve muscle soreness. In embodiments, disclosed A. muscaria compositions may increase metabolism, improve tissue formation, and/or improve immune health.

Other exemplary benefits of disclosed A. muscaria compositions include endurance and strength, calmness, relief from muscle soreness, and spiritual enlightenment. One exemplary tool to measure mental or emotional state, such as an improved state as provided by disclosed A. muscaria compositions is the Penn State Worry Questionnaire (PSWQ), which is the gold-standard self-report measure for pathological worry that shows effective psychometric properties among non-clinical and clinical populations (Gosselin et al., Encephale. 2001 September-October; 27(5): 475-84). An improvement in calmness or emotional state may be a numerical reduction of any amount in any of the questions asked.

c. Use to Promote Calmness and Reduce Stress

In some embodiments, improving health and wellness comprises eliciting or enhancing calmness in a subject. In some embodiments, the disclosed A. muscaria compositions are used to elicit or enhance calmness in a subject. In some embodiments, disclosed is use of A. muscaria compositions to elicit or enhance calmness. In some embodiments, disclosed is use of A. muscaria compositions in the manufacture of a medicament to elicit or enhance calmness. In some embodiments, administering the disclosed A. muscaria compositions to a subject elicits or enhances calmness in said subject.

In some embodiments, eliciting or enhancing calmness in a subject comprises a reduction in any of anxiety, worry, and hopelessness. In some embodiments, eliciting or enhancing calmness in a subject comprises promoting feelings of calm, tranquility, and a sense of peace. Physiological responses to eliciting or enhancing calmness in a subject may include a reduction in blood pressure and heart rate, as well as other signs of calmness known to those in the art

In some embodiments, improving health and wellness comprises reducing stress in a subject. In some embodiments, reducing stress comprises a reduction in any of anxiety, worry, and hopelessness. In some embodiments, reducing stress comprises promoting feelings of calm, tranquility, and a sense of peace. Physiological responses to a reduction in stress may include a reduction in blood pressure and heart rate, as well as other signs of a reduction of stress known to those in the art.

In some embodiments, the disclosed A. muscaria composition provides methods for improving a sense of calm and elicits feelings of comfortability, tranquility, and relaxation. Improvements in feeling a sense of calm may include a state of mind being free from agitation, excitement or disturbance. Additionally, improvements in a sense of calm may include improvements in emotional regulation and an increased sense of acceptance of self and of others (see Kraus et al., Social Indicators Research. 2008; 92, 169-181.; Juneau et al., Peerj. 2020; 1-19). Measurements of such will be readily understood and appreciated according to ordinary skill. Exemplary measures of improvements in sense of calm include the Equanimity Scale (the EQUA-S), Brief Mood Introspection Scale (BMIS) (Juneau et al., Peerj. 2020 1-19.; Mayer et al., Journal of Personality and Social Psychology. 1988; 102-111).

In some embodiments, improving health and wellness comprises promoting restorative sleep, including any of waking up feeling refreshed, increasing the amount of time spent in deep sleep, increasing the quantity of peaceful dreams, and reducing the amount of sleep necessary to feel refreshed, as well as other signs of a promotion of restorative sleep as will be known to those in the art. In some embodiments, improving health and wellness comprises preventing insomnia. In some embodiments, improving health and wellness comprises preventing insomnia. In some embodiments, improving health and wellness comprises reducing the severity of insomnia. The severity of insomnia may be determined, e.g., with use of The Insomnia Severity Index (ISI), a brief self-report instrument measuring the subject's perception of both nocturnal and diurnal symptoms of insomnia.

In some embodiments, improving health and wellness comprises any of a soothing of the body, a calming of the mind, and a reduction in physical distress; including feeling relaxed, at peace, and content; and a decrease in aches, pains, numbness, and tingling.

In some embodiments, improving health and wellness comprises includes any one or more of a reduction in feelings of nervousness, “jitters,” nervous tension, or anxiety; a reduction in feelings of malaise, unhappiness, existential angst, ennui, and general discontent; and an increase in feelings of wellbeing, wellness, relaxation, contentment, happiness, openness to experience, and life satisfaction.

d. Use as a Nootropic

In some embodiments, the A. muscaria composition provides methods of improving mental health and/or functioning, such as cognitive functioning. Improvements in mental health and functioning may include one or more of a reduction of neuroticism or psychological defensiveness, an increase in creativity or openness to experience, an increase in decision-making ability, an increase in feelings of wellness or satisfaction, or an increase in ability to fall or stay asleep. Additionally, improvements in mental health and functioning may include improvements in or a return to baseline in processing speed, learning and memory, autobiographical memory, shifting, and IQ. Measurements of such will be readily understood and appreciated according to ordinary skill. See, e.g., cognitive functioning aspects reviewed by Ahern & Semskova, Neuropsychology. 2017; 31(1):52-72. Exemplary measures of improvements of mental health and/or functioning include the Global Assessment of Functioning (GAF) scale, the Sleep Quality Scale (SQS) and other measures of sleep quality (see, e.g., Fabbri et al., Int J Environ Res Public Health. 2021; 18(3):1082, and the Social Functioning Scale (SFS) (see, e.g., Chan et al., Psychiatry Res. 2019; 276:45-55).

In some embodiments, the disclosed A. muscaria composition may be useful as a nootropic. Nootropics or smart drugs are well-known compounds or supplements that enhance cognitive performance. They work by increasing the mental function such as memory, creativity, motivation, and attention (Suliman et al., Evidence-Based Compl. and Alt. Med. 2016; 4391375: 1-12). Nootropic properties are present within the makeup of the disclosed A. muscaria compositions and, due to its cellular and molecular mechanisms of action, which enable a structural and functional plasticity, or synaptic plasticity, responsible for synaptic remodeling or known as cellular learning. In turn, nootropics mediate and enhance cognitive performance.

e. Use as a Meditation Aid

In certain embodiments, the disclosed A. muscaria composition is useful as an adjunct to meditation and/or meditative practice. Meditation refers to a variety of practices that focus on mind and body integration and are used to calm the mind and enhance overall well-being. While some forms of meditation involve maintaining mental focus on a particular sensation, other forms include the practice of mindfulness, which involves maintaining attention or awareness of the present moment without making judgements (see NCCIH NIH, “Meditation and Mindfulness: What You Need To Know” accessed 8/10/2022). The benefits of meditation include stress reduction, decreased anxiety, decreased depression, reduction in pain (both physical and psychological), improved memory, and increased efficiency. Physiological benefits include reduced blood pressure, heart rate, lactate, cortisol, and epinephrine, decreased metabolism and breathing pattern, oxygen utilization, and carbon dioxide elimination. Neurological and physiological correlates of meditation have been researched extensively in the past, and can be measured using electroencephalogram (EEG) (Sharma H., AYU. 2015; 36:233-7).

f. Use as a Psychotherapy Aid

In some embodiments, the A. muscaria composition is administered together with psychotherapy, such as psychosocial or behavioral therapy, including any of (or adapted from any of) cognitive behavioral therapy (e.g., as described in Arch. Gen. Psychiatry 1999; 56:493-502), interpersonal therapy (e.g., as described in Psychol Addict Behav 2009; 23(1): 168-174), contingency management based therapy (e.g., as described in Psychol Addict Behav 2009; 23(1): 168-174; in J. Consul. Clin. Psychol. 2005; 73(2): 354-59; or in Case Reports in Psychiatry, Vol. 2012, Article ID 731638), motivational interviewing based therapy (e.g., as described in J. Consul. Clin. Psychol. 2001; 69(5): 858-62), or meditation based therapy, such as transcendental meditation based therapy (e.g., as described in J. Consul. Clin. Psychol. 2000; 68(3): 515-52).

In some embodiments, “psychotherapy” is specifically “psychedelic-assisted psychotherapy.” Psychedelic-assisted psychotherapy, broadly, includes a range of related approaches that involve at least one session where the patient ingests a psychedelic and is monitored, supported, or otherwise engaged by one or more trained mental health professionals while under the effects of the psychedelic (see, e.g., Schenberg 2018). Protocols have been developed for the standardization of procedures which emphasize a high degree of care (see, e.g., Johnson 2008), such as the therapeutic approach used by MAPS to treat patients with PTSD using MDMA (e.g., as described in Mithoefer 2017).

In some embodiments, psychotherapy comprises any accepted modality of standard psychotherapy or counseling sessions, whether once a week, twice a week, or as needed; whether in person or virtual (e.g., over telemedicine or by means of a web program or mobile app); and whether with a human therapist or a virtual or AI “therapist.” As used herein, “therapist” refers to a person who treats a patient using the compositions and methods of the invention, whether that person is a psychiatrist, clinical psychologist, clinical therapist, registered therapist, psychotherapist, or other trained clinician, counselor, facilitator, or guide, although it will be understood that certain requirements will be appropriate to certain aspects of the drug-assisted therapy (e.g., prescribing, dispensing, or administering a drug, offering psychotherapeutic support). In some embodiments, a “person” may also include an AI.

In some embodiments, a patient will participate in a treatment protocol or a method of the invention, or be administered a composition of the invention as part of such a method, if the patient meets certain specified inclusion criteria, does not meet certain specified exclusion criteria, does not meet any specified withdrawal criteria during the course of treatment, and otherwise satisfies the requirements of the embodiment of the invention as claimed.

In some embodiments, disclosed A. muscaria compositions may be administered in conjunction with or as an adjunct to psychotherapy. In other embodiments, psychotherapy is neither necessitated nor desired, or no specific type of psychotherapy is necessitated or desired, however any of the disclosed methods can be used in combination with one or more psychotherapy sessions. The flexibility to participate in specific therapies, as well as to choose between any such therapies (or to decide to forgo any specific therapy), while still receiving clinically significant therapeutic effects, is among the advantages of the invention. Furthermore, a patient can participate in numerous other therapeutically beneficial activities, where such participation follows or is in conjunction with the administration of the composition, including breathing exercises, meditation and concentration practices, focusing on an object or mantra, listening to music, physical exercise, stretching or bodywork, journaling, grounding techniques, positive self-talk, or engaging with a pet or animal, and it should be understood that such participation can occur with or without the participation or guidance of a therapist.

g. Use to Alleviate Muscular Tension

In some embodiments, the improvement to health and wellness is an easing of muscular tension, including any of a reduction in soreness, tightness, aches, and pains; and an increase in flexibility and range of motion, as well as other signs of an easing of muscular tension as will be known to those in the art. In some embodiments, the disclosed A. muscaria compositions alleviate or prevent muscular tension, such as upon administration to a subject.

Muscle tension refers to the contraction of muscles without release for a period of time. The physiological mechanisms causing and maintaining muscle tension have not been fully uncovered. (Dieterich et al., Schmerz. 2022; 36:242-247). Even so, muscle tension is regarded as a complex condition, and generally considered to be a physiological response of the body to irritating thinking or stress. The implications of maintaining a constant or chronic state of contraction is that an individual may develop stress related disorders, or other bodily reactions such as tension or migraine headaches. (Liu et al., Complement Ther Clin Pract. 2020; 39:101132; American Psychological Association, “Stress Effects on the Body” 2018). The primary mechanism to counteract muscle tension is muscle relaxation which requires cortical activation, and not just a simple cessation of contraction. (Kato et al., Front Physiol. 2019; 10:1457). Electromyography on the skin can be used to assess muscular tension with a lower voltage number indicating a more relaxed muscle fiber. (Pluess et al., J Anxiety Disord. 2009; 23:1-11). In embodiments, the disclosed A. muscaria composition eases muscular tension allowing for muscle relaxation via cortical activation.

i. Muscle Spasms, Tightness, and Soreness

In some embodiments, the disclosed A. muscaria compositions alleviate or prevent one or more of muscle spasms, muscle tightness, and muscle soreness, such as upon administration to a subject. In certain embodiments pain in the form of muscle spasms can be treated with the disclosed A. muscaria composition. Muscle spasms are involuntary muscle contractions that are typically seen in the feet, calves, thighs, hands, and arms. The onset of a muscle spasm can persist from a few seconds to over 15 minutes. They can range from being mildly uncomfortable to causing acute pain. The exact physiological mechanisms causing muscle spasms may be unknown. Muscle spasms can have several causes such as dehydration, muscular tension, increased need for blood flow, strenuous exercise, or other underlying medical conditions. While they can be harmless, muscle spasms may also be an indicator of a more serious medical condition. Arteriosclerosis, or the narrowing of the arteries, multiple sclerosis, amyotrophic lateral sclerosis, or nerve compression on the lumbar spinal cord are examples of serious medical conditions that may accompany spasms. In severe cases clinicians could prescribe baclofen, or benzodiazepines to counteract muscle spasms, and may even advise exercise therapy. (Guo, Osmosis from Elsevier, “Muscle Spasms” 2021). The calming and reforming properties of the disclosed A. muscaria composition can be useful in easing muscle tension, and promoting healthy blood flow to treat muscle spasms both unrelated and related to underlying illness.

ii. Muscle Recovery

In some embodiments, the disclosed A. muscaria compositions promote muscle recovery, such as upon administration to a subject. Post-exercise recovery is the process of bodily restoration to an individual's basal conditions after exercise so that one can properly function out in the world as well as undergo additional training sessions without injury. Exercise typically results in the loss of bodily fluids and fuels, with the goal of post exercise recovery to restore homeostasis to the body. After exercise the cardiovascular, nervous, peripheral nervous, renal, thermoregulatory, endocrine, and immune systems can be impacted and disrupted due to the temporary stress put on the body. Post-exercise recovery is important to physical training ranging from low to high intensity. It can take a more pronounced role for individuals partaking in high intensity training regimes as the body's nutrients are depleted at a greater rate. Nutritional interventions such as the consumption of foods with vitamin D, and electrolyte drinks have been studied and used to promote post-exercise recovery. Physical intervention such as massage, hydrotherapy for the reduction of delayed onset muscle soreness, and sleep, regarded as a critical post-exercise recovery element, can also be integral to recovery. Peake, Curr Opin Psysiol. 2019; 10:17-26; dos Santos et al., Int J Environ Res Public Health. 2021; 18:5155.

Today, post-exercise recovery is often measured using specific biomarkers, and can be measured through blood sample analysis via the assessment of cortisol and creatine kinase levels in the body. There are also instances of post-exercise muscle biopsies that can uncover muscle protein synthesis and in turn determine the degree of recovery. Cintineo et al., Front Nutr. 2018; 5:83.

In embodiments, the disclosed A. muscaria composition may be greatly beneficial for post-exercise recovery because of its ability to ease pain, muscle tension, and its use as a sleep aid. The nutritional benefits of the mushroom should not be understated with an enrichment in vitamin D, niacin, copper, pantothenic acid, and phosphorus which lends a hand at replenishing the body's fuel post-workout.

iii. Menstrual Cramps

In some embodiments, the disclosed A. muscaria compositions are used to prevent or to alleviate menstrual cramps, such as upon administration to a subject. Dysmenorrhea or menstrual cramps are a common gynecological issue facing premenopausal women of reproductive age. During a menstrual period prostaglandins are contracted by the uterus to expel the uterine lining. Prostaglandins are lipids that cause the uterine muscle to contract, and if contraction of the uterine muscle is strong enough it can cut off a supply of oxygen to the muscle and subsequently cause pain. (Njoku et al., J Taibah Univ Med Sci. 2020; 16:93-101; Mayo Clinic, “Menstrual Cramps,” 2022). There are both primary and secondary versions of dysmenorrhea with the primary version being a common recurrence of pain that is not caused by disease and the secondary version being caused by reproductive organ issues. Endometriosis, adenomyosis, pelvic inflammatory disease, cervical stenosis and fibroids are all causes of secondary dysmenorrhea. Severity of dysmenorrhea appears to be measured through self-evaluation, questionnaires, and numerical pain scales. (Fang et al., Int J Obstet Anesth. 2021; 46:102961). The current embodiment may be useful in reducing or suppressing menstrual cramps because of its ability to improve health and wellness by reducing physical distress including aches and pains stemming from dysmenorrhea.

h. Use for Inducing Euphoria

In some embodiments, the disclosed A. muscaria compositions induce euphoria, such as upon administration to a subject. Euphoria includes a sense of pleasure characterized by strong feelings of happiness, excitement, and well-being. Those experiencing a sense of euphoria may describe it as being joyful and pleasurable, as well as feeling safe, secure, carefree, and supported (Cherry, What is a euphoric mood?, 2022). In some embodiments, a sense of euphoria is a euphoric mood. In some embodiments, a sense of euphoria is a transient feeling of euphoria. A sense of euphoria may include mild euphoria as well as greater relative feelings of euphoria. A sense of euphoria may include other signs as will be known to those in the art.

In some embodiments, the disclosed A. muscaria composition may produce a euphoric mood or feeling. Euphoria is a state of intense excitement and happiness, and is an amplification of pleasure. Bearn et al., Int Rev Neurobiol. 2015; 120: 205-33. Euphoria is considered to represent an abnormally extreme degree of happiness or contentment beyond which occurs in normal emotional response. (see https://dictionary.apa.org/euphoria). The feeling of euphoria may be measured by the MBG (morphine-benzedrine group or “euphoria”) Scale on the ARC Inventory. Manworren et al., J Psychoactive Drugs. 2021 Aug. 6:1-11. This assessment contains 16 True-False items relating to mood and surroundings. In some embodiments, the induction of a euphoric mood by the disclosed A. muscaria composition may be apparent in view of one or more of the presence of a pleasant feeling, contentment, feeling good, or openness.

i. Exemplary Pharmaceutical Use

In some aspects, the disclosed A. muscaria compositions are used to treat a condition, such as a disease or a disorder. In some embodiments, the disclosed A. muscaria compositions are used in the manufacture of a medicament to treat a condition, such as a disease or a disorder. In some embodiments, the disclosed A. muscaria compositions are administered to a subject having a condition, such as a disease or a disorder. In some embodiments, an effective amount of a disclosed A. muscaria composition is administered to a subject to treat a condition, such as a disease or disorder, in said subject. In some embodiments, disclosed are methods of treatment comprising administering a disclosed A. muscaria composition to a subject having a condition, such as a disease or disorder, thereby treating said condition.

“Treatment” covers any treatment of a disorder in a mammal, and particularly in a human, and includes: (a) preventing a disorder from occurring in a subject who may be predisposed to the disorder but has not yet been diagnosed with it: (b) inhibiting a disorder, i.e., arresting its development (including, e.g., prophylaxis); (c) relieving a disorder, i.e., causing regression of the disorder or its clinical symptoms; (d) protection from or relief of a symptom or pathology caused by or related to a disorder; (e) reduction, decrease, inhibition, amelioration, or prevention of onset, severity, duration, progression, frequency or probability of one or more symptoms or pathologies associated with a disorder; and (f) prevention or inhibition of a worsening or progression of symptoms or pathologies associated with a disorder. One will understand that a therapeutic amount necessary to effect treatment for purposes of this invention will, for example, be an amount that provides for objective indicia of improvement in patients having clinically-diagnosable symptoms. The effect may be prophylactic in terms of completely or partially preventing a disorder or symptom thereof and/or may be therapeutic in terms of a partial or complete cure for a disorder and/or adverse effect attributable to the disorder.

Herein, “therapeutic effect” or “therapeutic efficacy” means the responses(s) in a mammal, and preferably a human, after treatment that is judged to be desirable and beneficial. Depending on the disorder to be treated, or improvement in physiological or psychological functioning sought, and depending on the particular constituent(s) in the disclosed compositions under consideration, those responses shall differ, but would be readily understood by those of skill. For example, In some embodiments, “therapeutic effect” may refer to an effect caused by the disclosed composition, or its use in a method of the invention, such as the treatment of mental, sleep, and/or physical health disorders, and improvement in health and wellness.

In embodiments, measures of therapeutic effect include outcome measures (primary or secondary), endpoints, effect measures, and measures of effect within clinical or medical practice or research which can be used to assess an effect (positive and/or negative) of an intervention or treatment, whether patient-reported (e.g., questionnaires); based on other patient data (e.g., patient monitoring); gathered through laboratory tests such as from blood or urine; through medical examination by a doctor or other medical professional, or by digital means, such as by using electronic tools such as online tools, smartphones, wireless devices, biosensors, or apps.

j. Mental Health Disorders

In some aspects, provided are methods of using the disclosed A. muscaria compositions to treat a mental health disorder. In some embodiments, the disclosed A. muscaria compositions are used in the manufacture of a medicament to treat a mental health disorder. In some embodiments, the disclosed A. muscaria compositions are administered, e.g., in a therapeutically effective amount, to a subject having a mental health disorder to treat said mental health disorder.

Herein, “mental health disorder” refers to a disease condition in a mammal, and preferably in a human, that generally involves negative changes in emotion, mood, thinking, and/or behavior. For instance, other classifications and examples of mental health disorders include those disclosed in Merck Manual of Diagnosis and Therapy, 20th Ed. (2018), i.e., anxiety and stressor-related disorders, dissociative disorders, eating disorders, mood disorders, obsessive-compulsive and related disorders, personality disorders, schizophrenia and related disorders, sexuality, gender dysphoria, and paraphilias, somatic symptom and related disorders, suicidal behavior and self-injury, and substance-related disorders, which includes substance-induced and substance use disorders.

In some embodiments, the mental health disorder is any of depression, dysthymia, anxiety and phobia disorders (including generalized anxiety, social anxiety, panic, post-traumatic stress and adjustment disorders), feeding and eating disorders (including binge eating, bulimia, and anorexia nervosa), other binge behaviors, body dysmorphic syndromes, alcoholism, tobacco abuse, drug abuse or dependence disorders, disruptive behavior disorders, impulse control disorders, gaming disorders, gambling disorders, memory loss, dementia of aging, attention deficit hyperactivity disorder, personality disorders (including antisocial, avoidant, borderline, histrionic, narcissistic, obsessive compulsive, paranoid, schizoid and schizotypal personality disorders), attachment disorders, autism, and dissociative disorders, as well as such other mental health disorders as will be readily apparent to those of skill.

A mental health disorder, where otherwise undefined, will be understood to refer to the disorder as defined in the Diagnostic and Statistical Manual of Mental Disorders, 5th Edition (DSM-5). Although such terms generally shall refer to the criteria in the DSM-5, or a patient with a diagnosis based thereon, it will be appreciated that the compositions and methods of the invention are equally applicable to patients having the equivalent underlying disorder, whether that disorder is diagnosed based on the criteria in DSM-5 or in DSM-IV, whether the diagnosis is based on other clinically acceptable criteria, or whether the patient has not yet had a formal clinical diagnosis. Improvements and reductions in symptoms of an anxiety disorder are available to one of skill, e.g., by reference to the DSM-5.

k. Anxiety Disorders

In some embodiments, the mental health disorder is an anxiety disorder. In some embodiments, the disclosed A. muscaria compositions are used to treat an anxiety disorder. In some embodiments, the disclosed A. muscaria compositions are used in the manufacture of a medicament to treat an anxiety disorder. In some embodiments, the disclosed A. muscaria compositions are administered, e.g., in a therapeutically effective amount, to a subject having an anxiety disorder to treat said anxiety disorder. Anxiety disorders include a variety of psychological disorders that involve excess fear, worry, avoidance, and compulsive behaviors.

In some embodiments, the anxiety disorder is any of acute stress disorder, anxiety due to a medical condition, generalized anxiety disorder, panic disorder, panic attack, a phobia, post traumatic stress disorder (PTSD), separation anxiety disorder, social anxiety disorder, substance-induced anxiety disorder, and selective mutism.

In some embodiments, “anxiety disorder” refers to a state of apprehension, uncertainty, and/or fear resulting from the anticipation of an event and/or situation. An anxiety disorder can disrupt the physical and psychological functions of a person. These disruptions can cause a small hindrance to a debilitating handicap for a person's everyday life. An anxiety disorder can cause a physiological symptom, e.g., muscle tension, heart palpitations, sweating, dizziness, shortness of breath, etc. An anxiety disorder can also cause a psychological symptom, e.g., fear of dying, fear of embarrassment or humiliation, fear of an event occurring, etc.

In some instances, an anxiety disorder comprises a medical diagnosis based on the criteria and classification from the Diagnostic and Statistical Manual of Medical Disorders, 5th Ed. In some instances, an anxiety disorder comprises a medical diagnosis based on an independent medical evaluation. In some instances, an anxiety disorder comprises a medical diagnosis based on a self evaluation. Improvements and reductions in symptoms of an anxiety disorder are available to one of skill, e.g., by reference to the DSM-5.

l. Substance Use Disorders

In some embodiments, a mental health disorder comprises a substance use disorder. In some embodiments, the disclosed A. muscaria compositions are used to treat a substance use disorder. In some embodiments, the disclosed A. muscaria compositions are used in the manufacture of a medicament to treat a substance use disorder. In some embodiments, the disclosed A. muscaria compositions, e.g., in a therapeutically effective amount, are administered to a subject having a substance use disorder to treat said substance use disorder.

In some embodiments, the disclosed A. muscaria compositions prevent cravings for a substance. In some embodiments, the disclosed A. muscaria compositions reduce cravings for a substance. In some embodiments, the disclosed A. muscaria compositions facilitate cessation of substance use. In some embodiments, the disclosed A. muscaria compositions reduce one or more symptoms of substance use disorder.

In some embodiments, the substance use disorder is alcohol use disorder. In some embodiments, the substance use disorder is Cannabis use disorder. In some embodiments, the substance use disorder is hallucinogen use disorder. In some embodiments, the substance use disorder is inhalant use disorder. In some embodiments, the substance use disorder is opioid use disorder. In some embodiments, the substance use disorder is sedative use disorder. In some embodiments, the substance use disorder is stimulant use disorder. In some embodiments, the substance use disorder is tobacco use disorder or nicotine use disorder. Symptoms and signs of improvement for such disorders are available to one of skill, such as by reference to the DSM-5.

Substance use disorders (SUDs) occur when the recurrent use of alcohol and/or drugs causes clinically and functionally significant impairment, such as health problems, disability, and failure to meet major responsibilities at work, school, or home. According to the DSM-5, a diagnosis of substance use disorder is based on evidence of impaired control, social impairment, risky use, and pharmacological criteria. The DSM-5 establishes nine types of “substance-related” disorders: 1. Alcohol, 2. Caffeine (however, “substance use” disorder does not apply to caffeine), 3. Cannabis (e.g., marijuana), 4. Hallucinogens, 5. Inhalants, 6. Opioids (e.g., heroin), 7. Sedatives, Hypnotics, or Anxiolytics (e.g., benzodiazepines, barbiturates), 8. Stimulants (e.g., cocaine, methamphetamine), and 9. Tobacco. According to the DSM-5, each specific substance (other than caffeine) is addressed as a separate use disorder (i.e., alcohol use disorder, Cannabis use disorder, hallucinogen use disorder, inhalant use disorder, opioid use disorder, sedative use disorder, stimulant use disorder, tobacco use disorder, and nicotine use disorder), but nearly all substances are diagnosed based on the same overarching criteria.

The symptoms of SUDs include: 1. substance is often taken in larger amounts or over a longer period of time than was intended; 2. persistent desire or unsuccessful efforts to cut down or control substance use; 3. great deal of time is spent in activities necessary to obtain the substance, use the substance, or recover from its effects; 4. craving or strong desire to use the substance; 5. recurrent use resulting in failure to fulfill major role obligations at work, school, home; 6. continued substance use despite having persistent or recurrent social or interpersonal problems; 7. important social, occupational, or recreational activities are given up or reduced because of substance use; 8. recurrent substance use in situations in which it is physically hazardous; 9. substance use is continued despite knowledge of having a persistent or recurrent physical or psychological problem that is likely to have been caused or exacerbated by the substance; 10. tolerance, as defined by either of the following: a. a need for markedly increased amounts of the substance to achieve intoxication or desired effect, and b. a markedly diminished effect with continued use of the same amount of substance; and 11. withdrawal, as manifested by either of the following: a. characteristic withdrawal syndrome for the substance, or b. use of the substance or closely related substance is taken to relieve or avoid withdrawal symptoms.

The severity of the disorder is based on the number of symptoms exhibited: mild substance use disorder requires two to three symptoms, four or five symptoms indicate moderate substance use disorder, and greater than six symptoms indicates severe substance use disorder. In some embodiments, a substance use disorder will involve addiction. “Addiction” refers to a physical and/or psychological dependence on a substance, activity, and/or habit.

m. Behavioral Addictions

In some embodiments, a mental health disorder comprises a behavioral addiction. In some embodiments, the disclosed A. muscaria compositions are used to treat a behavioral addiction. In some embodiments, the disclosed A. muscaria compositions are used in the manufacture of a medicament to treat a behavioral addiction. In some embodiments, the disclosed A. muscaria compositions, e.g., in a therapeutically effective amount, are administered to a subject having a behavioral addiction to treat said behavioral addiction. In some embodiments, the disclosed A. muscaria compositions prevent the symptoms of a behavioral addiction. In some embodiments, the disclosed A. muscaria compositions reduce the symptoms of a behavioral addiction. In some embodiments, the disclosed A. muscaria compositions facilitate cessation of a behavioral addiction. In some embodiments, the disclosed A. muscaria compositions reduce one or more symptoms of a behavioral addiction. Symptoms and signs of prevention or reduction thereof are known to one of skill, including by reference to the DSM-5.

In some embodiments, the behavioral addiction is gambling disorder. In some embodiments, the behavioral addiction is gaming disorder. In some embodiments, the behavioral addiction is sexual addiction. In some embodiments, the behavioral addiction is compulsive buying disorder. In some embodiments, the behavioral addiction is technology addiction.

In some embodiments, the disclosed A. muscaria compositions reduce compulsive behaviors. Compulsive behaviors are described, e.g., in World Health Organization. International Classification of Diseases for Mortality and Morbidity Statistics, 11th Revision, 2018 (ICD-11).

n. Physical Health Conditions

In some aspects, provided are methods of using the disclosed A. muscaria compositions to treat physical health conditions. In some embodiments, the disclosed A. muscaria compositions are used to treat a physical health condition. In some embodiments, the disclosed A. muscaria compositions are used in the manufacture of a medicament to treat a physical health condition. In some embodiments, the disclosed A. muscaria compositions, e.g., in a therapeutically effective amount, are administered to a subject having a physical health condition to treat said physical health condition.

o. CNS Disorder or Neurodegenerative Condition

In some embodiments, a physical health condition comprises a CNS disorder. In some embodiments, a physical health condition comprises a neurodegenerative condition. In some embodiments, the CNS disorder or neurodegenerative condition is mediated by viral infection. In some embodiments, the disclosed A. muscaria compositions are used to treat a CNS disorder or neurodegenerative condition. In some embodiments, the disclosed A. muscaria compositions are used in the manufacture of a medicament to treat a CNS disorder or neurodegenerative condition. In some embodiments, the disclosed A. muscaria compositions, e.g., in a therapeutically effective amount, are administered to a subject having a CNS disorder or neurodegenerative condition to treat said CNS disorder or neurodegenerative condition. In some embodiments, a disclosed A. muscaria composition prevents or treats, such as alleviates, a CNS disorder or neurodegenerative condition mediated by viral infection.

Neurodegeneration may be assessed, e.g., by measuring markers of neuronal loss, such as cerebrospinal fluid markers, e.g., visinin-like protein 1 (VILIP-1), tau, and p-tau181 (Tarawneh et al., Neurol. 2015; 72(6): 656-665). Cognitive decline may also be used as a measure of neurodegeneration. Methods for assessing cognitive decline, e.g., comprehensive neuropsychological testing, are known to one of skill in the art. Exemplary cognitive evaluations include Mini-Mental State Examination (MMSE) and Montreal Cognitive Assessment (MoCA). See, e.g., Toh et al., Transl Neurodegener. 2014; 3:15. Cognitive decline and the progression of disease state may also be assessed using a condition-specific measure, e.g., the Unified Huntington's Disease Rating Scale (UHDRS).

Neurodegenerative conditions, such as diseases or disorders include, e.g., dementia, Alzheimer's disease, Huntington's disease, multiple sclerosis, and Parkinson's disease. A feature of neurodegenerative conditions is neuronal cell death, which, among other aspects, has been implicated in the promotion of inflammation. See, e.g., Chan et al., Annu Rev Immunol. 2015; 33: 79-106 and Chi et al., Int J Mol Sci. 2018; 19(10):3082. Neurodegenerative diseases can be classified according to primary clinical features, e.g., dementia, parkinsonism, or motor neuron disease, anatomic distribution of neurodegeneration, e.g., frontotemporal degenerations, extrapyramidal disorders, or spinocerebellar degenerations, or principal molecular abnormality (Dugger & Dickson, Cold Spring Harb Perspect Biol. 2017; 9(7):a028035.

p. Pain and Pain Disorders

In some embodiments, a physical health condition comprises pain, e.g., a pain disorder. In some embodiments, the disclosed A. muscaria compositions are used to treat a pain disorder. In some embodiments, the disclosed A. muscaria compositions are used in the manufacture of a medicament to treat a pain disorder. In some embodiments, the disclosed A. muscaria compositions, e.g., in a therapeutically effective amount, are administered to a subject having a pain disorder to treat said pain disorder.

In some embodiments, pain can be any of acute pain, chronic pain, neuropathic pain, and/or procedural pain. “Acute pain” refers to sudden pain from a specific cause (injury, infection, inflammation, etc.) that has lasted for a limited period of time (as opposed to chronic pain). “Chronic pain” refers to a persistent state of pain, and is often associated with long-term incurable or intractable medical conditions or diseases. “Neuropathic pain” refers to pain caused by damage or injury to nerves that innervate the skin, muscles, and/or other parts of the body. “Procedural pain” refers to pain arising from a medical, dental surgical, or other procedure which may for example be associated with an acute trauma.

Pain disorders include any disease, disorder, or condition associated with or caused by pain. Pain disorders also include conditions or disorders which are secondary to disorders such as chronic pain and/or neuropathic pain (i.e., are influenced or caused by a disorder such as chronic pain and/or neuropathic pain). Examples of such conditions include vasodilation and hypotension; conditions which are behavioral; or conditions in which detrimental effect(s) are the result of separate disorders or injuries, e.g., spinal cord injuries.

In some embodiments, the pain disorder is any of arthritis, allodynia, atypical trigeminal neuralgia, trigeminal neuralgia, somatoform disorder, hypoesthesis, hypealgesia, neuralgia, heuritis, neurogenic pain, analgesia, anesthesia dolorosa, causlagia, sciatic nerve pain disorder, degenerative joint disorder, fibromyalgia, visceral disease, chronic pain disorders, migraine/headache pain, chronic fatigue syndrome, complex regional pain syndrome, neurodystrophy, plantar fasciitis, or pain associated with cancer.

The International Association for the Study of Pain (IASP) defines pain as “an unpleasant sensory and emotional experience associated with actual or potential tissue damage, or described in terms of such damage.” Pain, such as chronic pain, and improvements thereof, such as a reduction of symptoms, may be measured according to known methods, e.g., by subject reporting, pain diaries, pain scales, applicable questionnaires (assessments of chronic pain and its impact on physical, emotional and social functions), ecological momentary assessments and computerized versions thereof. See, e.g., Salaffi et al., Best Practice & Research Clinical Rheumatology, 2015; 29(1):164-186 and Hawker et al., Arthritis Care Res (Hoboken). 2011; 63 Suppl 11:5240-52. Exemplary questionnaires include the Visual Analog Scale for Pain (VAS Pain), Numeric Rating Scale for Pain (NRS Pain), McGill Pain Questionnaire (MPQ), Short-Form McGill Pain Questionnaire (SF-MPQ), Chronic Pain Grade Scale (CPGS), Short Form-36 Bodily Pain Scale (SF-36 BPS), and Measure of Intermittent and Constant Osteoarthritis Pain (ICOAP), Migraine Diagnosis Questionnaire, the Migraine-Screen Questionnaire (MS-Q), the Fibromyalgia Survey Questionnaire (FSQ).

q. Inflammation and Inflammatory Disorders

In some embodiments, a physical health condition comprises inflammation, e.g., an inflammatory disorder. In some embodiments, the disclosed A. muscaria compositions are used to treat an inflammatory disorder. In some embodiments, the disclosed A. muscaria compositions are used in the manufacture of a medicament to treat an inflammatory disorder. In some embodiments, the disclosed A. muscaria compositions, e.g., in a therapeutically effective amount, are administered to a subject having an inflammatory disorder to treat said pain disorder and/or said inflammatory disorder.

In some embodiments, an inflammatory disorder is a disorder that causes acute inflammation, or that exhibits chronic inflammation as a symptom, including any of pressure ulcers, including acne vulgaris; oxalic acid/heartburn, age-related macular degeneration (AMD), allergies, allergic rhinitis, Alzheimer's disease, amyotrophic lateral sclerosis, Anemia, appendicitis, arteritis, arthritis, including osteoarthritis, rheumatoid arthritis, juvenile idiopathic arthritis, spondyloarthropathy such as ankylosing spondylitis, reactive arthritis (Reiter syndrome), psoriatic arthritis, enteroarthritis associated with inflammatory bowel disease, Whipple and Behcet's disease, septic arthritis, gout (also known as gouty arthritis, crystalline synovitis, metabolic arthritis), pseudogout (calcium pyrophosphate deposition disease), and Still's disease. Arthritis can affect a single joint (monoarthritis), two to four joints (oligoarthritis), or five or more joints (polyarthritis); asthma, atherosclerosis, autoimmune disorder, balanitis, blepharitis, bronchiolitis, bronchitis, bullous pemphigoid, burns, bursitis, cancer, including NF-κB-induced inflammatory cancer; cardiovascular disease, including hypertension, endocarditis, myocarditis, heart valve dysfunction, congestive heart failure, myocardial infarction, diabetic heart abnormalities, vascular inflammation, including arteritis, phlebitis, and vasculitis; arterial occlusive disease, including arteriosclerosis and stenosis; inflammatory cardiac hypertrophy, peripheral arterial disease, aneurysm, embolism, incision, pseudoaneurysm, vascular malformation, vascular nevus, thrombosis, thrombophlebitis, varicose veins, stroke, cardiac arrest, and carditis; celiac disease, cellulitis, cervicitis, cholangitis, cholecystitis, chorioamnionitis, chronic obstructive pulmonary disease (COPD), cirrhosis, congestive heart failure, conjunctivitis, colitus, cyclophosPHamide-induced cystitis, cystic fibrosis, cystitis, cold, lacrimal inflammation, dementia, dermatitis, including atopic dermatitis, chronic photosensitivity dermatitis, eczema, atopic eczema, contact eczema, dryness eczema, seborrheic eczema, sweating disorders, discoid eczema, venous eczema, herpetic dermatitis, neurodermatitis, and autosensitizing dermatitis, stasis dermatitis, purulent sweaty, lichen planus, psoriasis, including psoriasis vulgaris, nail psoriasis, prickly psoriasis, scalp psoriasis, inverse psoriasis, pustular psoriasis, erythrodermic psoriasis, and psoriatic arthritis; rosacea, and scleroderma, including morphea; pharmacologically induced inflammation, including from legal or illegal drugs, and chemicals; chronic neurogenic inflammation, including primary and secondary neural inflammation; dermatomyositis, diabetes, diabetic neuropathy, diabetic retinopathy, diabetic nephropathy, diabetic ulcer, digestive system disease, emphysema, encephalitis, endocarditis, endometritis, enterocolitis, epicondylitis, epididymis, fasciitis, fibromyalgia, fibrosis, connectitis, gastritis, gastroenteritis, gingivitis, glomerulonephritis, glossitis, heart disease, heart Valvular dysfunction, hepatitis, purulent spondylitis, Huntington's disease, hyperlipidemic pancreatitis, hypertension, ileitis, infection, including lymphangitis, lymphadenitis, bacterial cystitis, bacterial encephalitis, pandemic influenza, viral encephalitis, and viral hepatitis (types A, B, and C); inflammatory bowel disease, including Chron's disease; inflammatory heart enlargement, inflammatory neuropathy, insulin resistance, between Interstitial cystitis, interstitial nephritis, iritis, ischemia, ischemic heart disease, keratitis, keratoconjunctivitis, laryngitis, lupus nephritis, mastitis, mastoiditis, meningitis, metabolic syndrome (syndrome X), migraine, multiple sclerosis, myelitis, myocarditis, myositis, nephritis, non-alcoholic steatohepatitis, obesity, umbilitis, ovitis, testitis, osteochondritis, osteopenia, osteomyelitis, osteoporosis, osteomyelitis, otitis, pancreatitis, Parkinson's disease, parotitis, pelvic inflammatory disease, pemphigus vulgaris, pericarditis, Peritonitis, pharyngitis, phlebitis, pleurisy, interstitial pneumonia, polycystic nephritis, polymyositis, proctitis, prostatitis, psoriasis, pulpitis, pyelonephritis, portal vein, renal failure, reperfusion injury, retinitis, rheumatic fever Rhinitis, fallopianitis, sarcoidosis, salivary glanditis, sepsis, including bacteremia and viremia; sinusitis, spastic colon, stenosis, stomatitis, stroke, surgical complications, synovitis, tendonitis, tendonitis, tendonitis, thrombophlebitis, tonsillitis, trauma, traumatic brain injury, graft rejection, including graft versus host disease (GVHD); a Th1-mediated inflammatory disease, trigonitis, tuberculosis, tumor, urethritis, bursitis, uveitis, vaginitis, vasculitis, including Buerger's disease, cerebral vasculitis, Churg-Strauss arteritis, cryoglobulinemia, essential cryoglobulin vasculitis, giant cells arteritis, golfer vasculitis, Henoch-Schonlein purpura, hypersensitivity vasculitis, Kawasaki disease, microscopic polyarteritis/polyvasculitis, nodular polyarteritis, rheumatoid polymuscular muscle pain (PMR), rheumatic vasculitis, Takayasu arteritis, Wegener's granulomatosis, systemic lupus erythematosus (SLE), relapsing polychondritis, Behcet's disease; ulcerative colitis such as ulcerative proctitis, left side colitis, total colitis, and fulminant colitis; and vulvitis.

In some embodiments, chronic inflammation includes tissue inflammation such as, e.g., skin inflammation, muscle inflammation, tendon inflammation, ligament inflammation, bone inflammation, cartilage inflammation, lung inflammation, heart inflammation, liver inflammation, pancreatic inflammation, kidney inflammation, bladder inflammation, gastric inflammation intestinal inflammation, neuroinflammation, and brain inflammation.

In some embodiments, the disclosed A. muscaria compositions are used to reduce inflammation. In some embodiments, the disclosed A. muscaria compositions are used in the manufacture of a medicament to reduce inflammation. In some embodiments, the disclosed A. muscaria compositions, e.g., in a therapeutically effective amount, are administered to a subject to reduce inflammation.

A reduction in inflammation, such as chronic systemic inflammation, may be measured according to various methods available to one of skill. Inflammatory biomarkers may be detected from biological specimens, for example, a subject's blood, such as plasma or serum, or saliva. In one example, inflammation may be detected by measuring high-sensitivity C-reactive protein (CRP) and white blood cell count from a blood test. CRP may also be detected in a saliva sample. Salivary CRP is not synthesized locally in the mouth and may reflect more systemic levels of inflammation compared to other inflammatory biomarkers, such as cytokines (Szabo & Slavish, Psychoneuroendocrinology. 202; 124:105069). Additionally clinical pathology data, e.g., hematology data on erythrocyte parameters, platelet count, total number of leukocytes, and leukocyte differentials and morphology, coagulation data on clotting times and fibrinogen, and clinical chemistry data on total protein, albumin and globulin, liver enzymes, renal parameters, electrolytes, and bilirubin can provide an initial indication of the presence and potentially the location of inflammation, in the absence of specific data on immune tissues. See, e.g., Germolec et al., Methods Mol Biol. 2018; 1803:57-79 and Luo et al., Clin Lab. 2019 1; 65(3).

r. Sleep Disorders

In some embodiments, a physical health condition comprises a sleep disorder. In some embodiments, the disclosed A. muscaria compositions are used to treat a sleep disorder. In some embodiments, the disclosed A. muscaria compositions are used in the manufacture of a medicament to treat a sleep disorder. In some embodiments, the disclosed A. muscaria compositions, e.g., in a therapeutically effective amount, are administered to a subject having a sleep disorder to treat said sleep disorder. In some embodiments are methods of treating health conditions, wherein the health condition is a sleep disorder. In some embodiments, the sleep disorder may or may not be comorbid with one or more mental health disorders.

The Diagnostic Classification of Sleep and Arousal Disorders (DSCAD) classifies sleep disorders broadly into nine categories: (1) psychophysiological insomnia; (2) sleep disorders associated with mental disorders; (3) sleep disorders associated with a regular use of drugs and alcohol; (4) insomnias associated with sleep-induced breathing disorders; (5) sleep disorders associated with nocturnal myoclonus and restless legs syndrome (RLS); (6) sleep disorders by other disorders, drugs, and environmental conditions; (7) childhood onset insomnias; (8) other types of insomnia; and (9) sleep abnormalities with no symptoms of insomnia. Each of these categories is based on the state of disorder.

The International Classification of Sleep Disorders (ICSD) classifies sleep disorders broadly into four categories: (1) dyssomnias comprising disorders that are primarily disorders of sleep per se [for example, intrinsic sleep disorders such as narcolepsy, extrinsic sleep disorders, and circadian rhythm sleep disorders]; (2) parasomnias comprising disorders of abnormal behaviors that occur during sleep (also known as abnormal behavior during sleep) [arousal disorders, sleep-wake transition disorders, parasomnias usually associated with REM sleep, and other parasomnias]; (3) sleep disorders associated with medical/psychiatric disorders [sleep disorders associated with mental disorders, sleep disorders associated with neurologic disorders, and sleep disorders associated with other medical disorders]; and (4) proposed sleep disorders, for example, short sleeper, long sleeper, subwakefulness syndrome, and the like. To this date, this classification includes about 90 categories of sleep disorders. Currently, further classifications have been made continuously based on etiology.

According to the International Classification of Diseases, Tenth Edition (ICD-10) (1992), which have been published by World Health Organization (WHO), sleep disorders are classified into (1) nonorganic sleep disorders (F51: for example, nonorganic insomnia, nonorganic hypersomnia, sleep walking, sleep terrors, nonorganic disorder of the sleep-wake schedule, nightmares, and the like); (2) sleep disorders [G47: for example, sleep apnoea, disorders of initiating and maintaining sleep (insomnias), disorders of excessive somnolence (hypersomnia), cataplexy, narcolepsy, cataplexy attacks, disorders of the sleep-wake schedule (such as irregular sleep pattern, sleep rhythm disorder, and delayed sleep phase syndrome), and the like]; (3) other respiratory conditions originating in the perinatal period (P28: for example, primary sleep apnoea of newborn, and the like); and (4) personal history of risk-factors, not elsewhere classified (Z91: e.g., personal history of unhealthy sleep-wake schedule, and the like).

The Merck Manual, 17th ed., Section 14, Chapter 173 defines sleep disorders as disorders that affect the ability to fall asleep, stay asleep, or stay awake or that produce sleep-related abnormal behaviors, and the like. According to the Manual, sleep disorders are classified into the following symptoms: (1) insomnia (disorders falling asleep, difficulty staying asleep, or a disturbance in sleep patterns that causes inadequate sleep, and the like; for example, sleep-onset insomnia (difficulty falling asleep), early morning awakening, sleep-wake reversals, rebound insomnia, and the like); (2) hypersomnia (defined as a pathological increase of at least 25% in total sleeping time; for example, narcolepsy, sudden episode of sleep, and the like); (3) sleep apnoea syndromes, parasomnias (based on patients' chief complaints); and the like.

Herein, when referring to sleep disorders, one will understand the term to encompass, unless otherwise defined, any of the above definitions (including as since revised) and such other generally accepted definitions known to those in the art. In clinical treatment, sleep disorders are often broadly classified into (1) insomnias, (2) hypersomnia, (3) parasomnias, and (4) disorders of sleep-wake schedule (corresponding to the circadian rhythm sleep disorders of ICSD), made symptomatically based on patients' chief complaints, separately from the international classifications described above. Depending on etiology, a different medical treatment is given to each type of sleep disorder. In some embodiments, the sleep disorder is any of an insomnia, a hypersomnia, a parasomnia, and a disorder of sleep-wake schedule.

In some embodiments, the disclosed A. muscaria compositions are used to treat sleep disorders such as, but not limited to, insomnia disorders, sleep-related breathing disorders, central disorders of hypersomnolence, circadian rhythm sleep-wake disorders, sleep-related movement disorders, parasomnias, non-24-hour sleep wake disorder, excessive daytime sleepiness, shift work disorder, and others. In some embodiments, the disclosed A. muscaria compositions are used in the manufacture of a medicament to treat sleep disorders such as, but not limited to, insomnia disorders, sleep-related breathing disorders, central disorders of hypersomnolence, circadian rhythm sleep-wake disorders, sleep-related movement disorders, parasomnias, non-24-hour sleep wake disorder, excessive daytime sleepiness, shift work disorder, and others. See, e.g., Sateia, Chest. 2014; 146(5):1387-1394. Regular sleep and sleep quality is associated with enhanced longevity (Dew et al., Psychosomatic Medicine, 2003; 65(1):63-73; Kripke et al., Arch Gen Psychiatry. 1979; 36(1):103-16; Mazzotti et al., Front Aging Neurosci. 2014; 6: 134).

r. Insomnia Disorders

In some embodiments, the disclosed A. muscaria compositions are used to treat insomnia. In some embodiments, the disclosed A. muscaria compositions are used in the manufacture of a medicament to treat insomnia. Broadly, insomnia is defined as a persistent difficulty with sleep initiation, duration, consolidation, or quality, by the ICSD-3 manual. Chronic insomnia is characterized by persistent insomnia that occurs at least three times per week for at least three months, while the same symptoms for less than three months is termed short-term insomnia.

Regardless of whether an individual is diagnosed with chronic, or short-term insomnia, most instances of the disorder present as either sleep-onset insomnia, or sleep maintenance insomnia, wherein the former is characterized by a difficulty falling asleep, while those suffering with the latter experience trouble staying asleep. Criteria for diagnosing insomnia include (1) a report of sleep initiation or maintenance problems, (2) adequate opportunity and circumstances to sleep, and (3) daytime consequences (Sateia, Chest. 2014; 146(5):1387-1394).

There are multiple factors that may cause, or exasperate symptoms of insomnia; however, stress and anxiety are each significant contributing factors. The anxiolytic effects of the disclosed A. muscaria compositions, including the promotion of calmness and reduction of stress, are effective in treating insomnia, including chronic insomnia and short-term insomnia characterized as either sleep-onset or sleep maintenance insomnia, by treating some of its underlying causes.

s. Circadian Rhythm Sleep-Wake Disorders

In some embodiments, the disclosed A. muscaria compositions are used to treat a circadian rhythm sleep-wake disorder. In some embodiments, the disclosed A. muscaria compositions are used in the manufacture of a medicament to treat a circadian rhythm sleep-wake disorder. Delayed sleep-wake phase disorder, advanced sleep-wake phase disorder, irregular sleep-wake rhythm disorder, non-24-h sleep-wake rhythm disorder, shift work disorder, and jet lag disorder are all classified as circadian rhythm sleep-wake disorders. Such disorders are diagnosed with the following criteria: (1) a chronic or recurrent pattern of sleep-wake rhythm disruption primarily caused by an alteration in the endogenous circadian timing system or misalignment between the endogenous circadian rhythm and the sleep-wake schedule desired or required, (2) a sleep-wake disturbance (ie, insomnia or excessive sleepiness, and (3) associated distress or impairment (Sateia, Chest. 2014; 146(5):1387-1394)

In one example, Non-24-Hour Sleep Wake disorder (N24SWD) refers to a condition wherein an individual's natural circadian rhythm is shorter or (more commonly) longer than 24 hours (Pacheco, Sleep Foundation, “Non-24-Hour Sleep Wake Disorder,” 2022). This causes such individuals to struggle with fluctuations in appetite, mood, and alertness that—while following their body's internal clock—is generally at odds with rising and setting of the sun. When heavily desynchronized, individuals with N24SWD exhibit a natural preference for sleeping during the day, and experience difficulty sleeping at night.

In some embodiments, the disclosed A. muscaria compositions are used to treat shift work disorder. In some embodiments, the disclosed A. muscaria compositions are used in the manufacture of a medicament to treat shift work disorder. Shift work disorder is generally caused by an activity (e.g., an obligatory obligation like work, as the name suggests) that forces an individual to stay awake when they would ordinarily be asleep. This causes individuals to lose, on average, between 1-4 hours of sleep each day, leading to lethargy, mood swings, low testosterone, and negatively impacting the ability to function while attempting to accomplish day-to-day tasks (Pacheco, Sleep Foundation, “Diagnosing Shift Work Disorder,” 2022).

t. Excessive Daytime Sleepiness

In some embodiments, the disclosed A. muscaria compositions are used to treat excessive daytime sleepiness. In some embodiments, the disclosed A. muscaria compositions are used in the manufacture of a medicament to treat excessive daytime sleepiness. As the name implies, excessive daytime sleepiness is characterized by abnormal levels of drowsiness during the day, and a desire to sleep (Pacheco, American Sleep Association, “Excessive Daytime Sleepiness: Causes, Test and Treatments,” 2022). Excessive daytime sleepiness may be caused by a combination of complex factors, but is generally due to a chronic lack of sleep, or fragmented, poor-quality sleep.

u. Immune Disorders

In some embodiments, a physical health condition comprises an immune disorder. In some embodiments, the disclosed A. muscaria compositions are used to treat an immune disorder. In some embodiments, the disclosed A. muscaria compositions are used in the manufacture of a medicament to treat an immune disorder. In some embodiments, the disclosed A. muscaria compositions, e.g., in a therapeutically effective amount, are administered to a subject having an immune disorder to treat said immune disorder. Autoimmune diseases can be divided into systemic and organ-specific autoimmune disorders according to the main clinical and pathological characteristics of each disease.

In some embodiments, immune disorders include disorders characterized by deregulation of Toll-like receptor signaling and/or type I interferon-mediated immunity. In some embodiments, the immune disorder is any of acne vulgaris, acute respiratory distress syndrome, Addison's disease, adrenocortical insufficiency, adrenogenital syndrome, allergic conjunctivitis, allergic rhinitis, allergic intraocular inflammatory diseases, ANCA-associated small-vessel vasculitis, angioedema, ankylosing spondylitis, aphthous stomatitis, arthritis, asthma, atherosclerosis, atopic dermatitis, Behcet's disease, Bell's palsy, berylliosis, bronchial asthma, bullous herpetiformis dermatitis, bullous pemphigoid, carditis, celiac disease, cerebral ischaemia, chronic obstructive pulmonary disease, cirrhosis, Cogan's syndrome, contact dermatitis, COPD, Crohn's disease, Cushing's syndrome, dermatomyositis, diabetes mellitus, discoid lupus erythematosus, eosinophilic fasciitis, epicondylitis, erythema nodosum, exfoliative dermatitis, fibromyalgia, focal glomerulosclerosis, giant cell arteritis, gout, gouty arthritis, graft-versus-host disease, hand eczema, Henoch-Schonlein purpura, herpes gestationis, hirsutism, hypersensitivity drug reactions, idiopathic cerato-scleritis, idiopathic pulmonary fibrosis, idiopathic thrombocytopenic purpura, inflammatory bowel or gastrointestinal disorders, inflammatory dermatoses, juvenile rheumatoid arthritis, laryngeal edema, lichen planus, Loeffler's syndrome, lupus nephritis, lupus vulgaris, lymphomatous tracheobronchitis, macular edema, multiple sclerosis, musculoskeletal and connective tissue disorder, myasthenia gravis, myositis, obstructive pulmonary disease, ocular inflammation, organ transplant rejection, osteoarthritis, pancreatitis, pemphigoid gestationis, pemphigus vulgaris, polyarteritis nodosa, polymyalgia rheumatica, primary adrenocortical insufficiency, primary billiary cirrhosis, pruritus scroti, pruritis/inflammation, psoriasis, psoriatic arthritis, Reiter's disease, relapsing polychondritis, rheumatic carditis, rheumatic fever, rheumatoid arthritis, rosacea caused by sarcoidosis, rosacea caused by scleroderma, rosacea caused by Sweet's syndrome, rosacea caused by systemic lupus erythematosus, rosacea caused by urticaria, rosacea caused by zoster-associated pain, sarcoidosis, scleroderma, segmental glomerulosclerosis, septic shock syndrome, serum sickness, shoulder tendinitis or bursitis, Sjogren's syndrome, Still's disease, stroke-induced brain cell death, Sweet's disease, systemic dermatomyositis, systemic lupus erythematosus, systemic sclerosis, Takayasu's arteritis, temporal arteritis, thyroiditis, toxic epidermal necrolysis, type-1 diabetes, ulcerative colitis, uveitis, vasculitis, and Wegener's granulomatosis.

In some embodiments, the immune disorder is an autoimmune disorder. In some embodiments, an autoimmune disorder is any of acute disseminated encephalomyelitis (ADEM), Addison disease, allergy or hypersensitivity, amyotrophic lateral sclerosis, antiphospholipid antibody syndrome (APS), arthritis, autoimmune disease, autoimmune hemolytic anemia, autoimmune hepatitis, autoimmune inner ear disease, autoimmune pancreatitis, bullous pemphigoid, celiac disease, Chagas disease, chronic obstructive pulmonary disease (COPD), type 1 diabetes (T1D), endometriosis, fibromyalgia, goodpasture's syndrome, Graves' disease, Guillain-Barre syndrome (GBS), Hashimoto's thyroiditis, suppurative spondylitis, idiopathic thrombocytopenic purpura, inflammatory bowel disease, interstitial cystitis, lupus, including discoid lupus erythematosus, drug-induced lupus lupus erythematosus, lupus nephritis, neonatal lupus, subacute cutaneous lupus erythematosus, and systemic lupus erythematosus; morphea, multiple hard Keratosis (MS), myasthenia gravis, myopathy, narcolepsy, neuromuscular angina, pemphigus vulgaris, pernicious anemia, primary biliary cirrhosis, recurrent diffuse encephalomyelitis, including polyphasic diffuse encephalomyelitis, rheumatic fever, schizophrenia, scleroderma, Sjogren's syndrome, tendonitis, vasculitis, and vitiligo.

In some embodiments, the autoimmune disorder is a systemic autoimmune disorder, such as systemic lupus erythematosus (SLE), Sjogren's syndrome, scleroderma, rheumatoid arthritis, and polymyositis. In some embodiments, the autoimmune disorder is a local autoimmune disorder, including those of the endocrine system, including type 1 diabetes, Hashimoto's thyroiditis, and Addison's disease; the cutaneous, including pemphigus vulgaris; the blood, including autoimmune hemolytic anemia; and the nervous system, including multiple sclerosis.

v. Sexual Dysfunction

In some embodiments, a physical health condition comprises a sexual disorder, such as a disorder characterized by sexual dysfunction. In some embodiments, the disclosed A. muscaria compositions are used to treat a sexual disorder, such as a disorder characterized by sexual dysfunction. In some embodiments, the disclosed A. muscaria compositions are used in the manufacture of a medicament to treat a sexual disorder, such as a disorder characterized by sexual dysfunction. In some embodiments, the disclosed A. muscaria compositions, e.g., in a therapeutically effective amount, are administered to a subject having a sexual disorder to treat said sexual disorder.

In some embodiments, the sexual disorder is delayed ejaculation and erectile disorder. In some embodiments, the sexual disorder is female orgasmic disorder. In some embodiments, the sexual disorder is female sexual interest or arousal disorder. In some embodiments, the sexual disorder is genito-pelvic pain/penetration disorder. In some embodiments, the sexual disorder is female hypoactive sexual desire disorder. In some embodiments, the sexual disorder is male hypoactive sexual desire disorder. In embodiments, the sexual disorder is premature ejaculation. In embodiments, the sexual disorder is substance/medication-induced sexual dysfunction.

Signs or symptoms of sexual dysfunction can be assessed and alleviation thereof determined according to methods known to one of skill. Such evaluations include interviewing, use of assessments and questionnaires, and physiological investigations (Bhugra & Colombini, Advances in Psychiatric Treatment, 2018; 19(1):48-55; Clayton, J Clin Psychiatry. 2001; 62 Suppl 3:5-9). In some embodiments, sexual function is assessed by evaluating, for example determining improvements in, one or more of sexual desire, sexual aversion, sexual enjoyment, sexual drive, genital response, such as vaginal dryness/impotence and male erectile disorder, orgasm disorders, such as orgasmic dysfunction and premature ejaculation, disorders of sexual pain, such as dyspareunia and vaginismus, and other forms of dysfunction.

The International Consultation on Sexual Medicine has proposed updated methods for diagnosing sexual dysfunction in men and women, with specific recommendations for sexual history taking and diagnostic evaluation. Standardized scales, checklists, and validated questionnaires are adjuncts that may be used to evaluate sexual dysfunction and sexual satisfaction (Hatzichristou et al., J Sex Med. 2016; 13(8):1166-8). Exemplary questionnaires include, for example, The Sexual Satisfaction Questionnaire, The Sexual Interest and Satisfaction Scale, The Sexual Satisfaction Scale, The Pinney Satisfaction Inventory, and the Female Sexuality Satisfaction Questionnaire.

### J. EXAMPLES

### Example 45: Assessment of the Effects of Exemplary A. muscaria Extract AME-1 on Microglia Exposed to Stimulatory Conditions

Purpose: The study described herein was completed to determine how exemplary aqueous A. muscaria extract AME-1 affects microglial function, particularly as it relates to cytokine production. As one of the primary immune cells in the brain, microglia are resident macrophages of the CNS. Microglia respond to a variety of stimuli, including bacterial components, e.g., lipopolysaccharide (LPS), viral components, e.g., polyinosinic-polycytidylic acid (poly I:C) nucleotides, and inflammatory cytokines, e.g., tumor necrosis factor (TNF).

Methods: Human microglial cell line HMC3 was stimulated with LPS, poly I:C, a double-stranded RNA that induces innate immunity in mammals, and TNF alone and in the presence of exemplary extract AME-1.

Additionally, the effects of AME-1 on pro-apoptotic cellular pathways, such as induction of caspase-3, were evaluated. Caspase-3 is a key mediator of apoptosis and facilitates proteolytic cleavage of proteins in the cell nucleus. The caspase 3 assay was performed according to manufacturer's instructions (abcam, Caspase-3 Assay Kit; colorimetric; ab39401). Briefly, the assay was based on spectrophotometric detection of the chromophore p-nitroaniline (p-NA) after cleavage from the labeled substrate DEVD-p-NA. The p-NA light emission was quantified using a spectrophotometer, and the absorbance of p-NA from an apoptotic sample with an untreated control facilitated determination of the fold increase in Caspase-3 activity.

### Results and Significance:

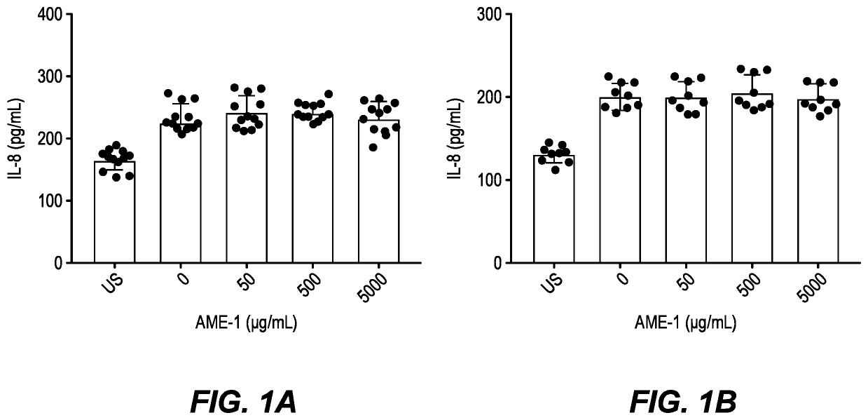

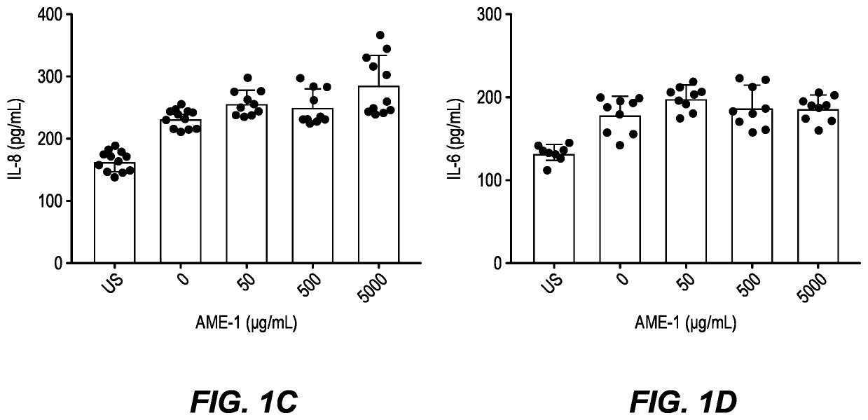

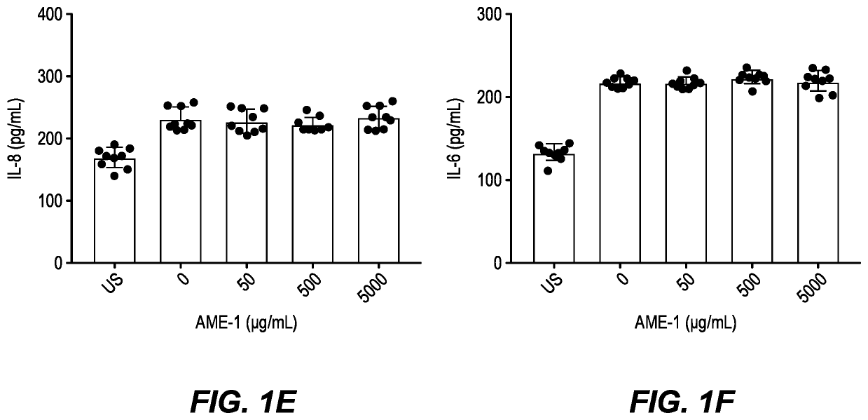

FIG. 1A and FIG. 1B show the production of IL-8 and IL-6, respectively, by LPS-activated HMC3 cells exposed to AME-1. FIG. 1C and FIG. 1D show production of IL-8 and IL-6, respectively, by polyL:C-induced HMC3 cells exposed to AME-1 (n=3, *=p<0.05, **=p<0.05). FIG. 1E and FIG. 1F show production of IL-8 and IL-6, respectively, by TNF-induced HMC3 cells exposed to AME-1.

AME-1 had no significant effect on the response of microglia to LPS and TNF. However, AME-1 potentiated poly I:C activation of microglial cells, resulting in greater production of the chemokine IL-8. IL-8 plays a role of attracting immune cells to the site of infection. Preliminary evidence shows that this may be mediated by AME-1 upregulating Toll-Like Receptor-3 (TLR3), which mediates microglial responses to poly I:C (data not shown).

In addition, AME-1 did not increase caspase-3 activity, and cell death was not observed following exposure to AME-1. Accordingly, AME-1 may safely enhance the antiviral immune response in the brain. Such activity may prevent or reduce viral infection of brain tissue. Viral infection of the brain, especially chronic viral infection, has been associated with neuroinflammation and neurodegenerative diseases. See, e.g., Front Neurosci. 2021; 15:648629.

### Example 46: Abuse Liability Assessment of Exemplary A. muscaria Extract AME-1

Purpose: A conditioned place preference (CPP) study was conducted in rats to evaluate the abuse potential of exemplary aqueous A. muscaria extract AME-1. The CPP study is a standard behavioral model used for studying the reward and aversive effects of a compound and is designed to determine the reinforcing properties of drugs of abuse using classical Pavlovian conditioning. Accordingly, the CPP paradigm provides for a rapid measurement of the positive reinforcing or aversive properties of a test sample, in this case, AME-1.

Methods: Four treatment groups of 8 male CD® [Crl:CD®(SD)] rats were exposed to unique environmental cues associated with a conditioned stimuli (CS+) or saline (CS−) in a 3-chamber straight alleyway system. Following a habituation phase (Week −1) during which compartment preference was determined for each animal, the conditioning phase (Week 1 and Week 2) was conducted during which compartments 1 and 3 were paired with the test and control articles or saline. Saline (CS−) and test/control articles (CS+, at 800 and 1100 mg/kg of exemplary A. muscaria aqueous extract AME-1 or 20 mg/kg cocaine) were administered via oral gavage at a dose volume of 5 mL/kg. After two sets of conditioning trials during the conditioning phase, each rat was retested for free-choice side preference (Days 5 and 12). Each rat received eight stimulus environmental pairing sessions (e.g., four test article and four saline) and two CPP test sessions (T1, T2). The study design was as follows:

| | | | CS+ | | | |
|---|---|---|---|---|---|---|
| | | | Dose | | | |
| | | CS+ | Concen- | | | |
| | | Dose | tration | Dose | | No. of |
| Group | | Level | Level | Volume | | Male |
| No. | CS+ | (mg/kg) | (mg/mL) | (mL/kg) | CS− | Animals |
| 1 | Saline | 0 | 0 | 5 | Saline | 8 |
| 2 | A. muscaria | 800 | 160 | 5 | Saline | 8 |
| | aqueous | | | | | |
| | extract | | | | | |
| 3 | A. muscaria | 1100 | 220 | 5 | Saline | 8 |
| | aqueous | | | | | |
| | extract | | | | | |
| 4 | Cocaine | 20 | 4 | 5 | Saline | 8 |

CS+ = Conditioned stimuli (test article or cocaine).
CS− = Aversive stimuli (saline).

The following parameters and endpoints were evaluated in this study: mortality, clinical signs, body weights, and CPP testing. During CPP testing sessions, the following parameters were assessed: 1) Total time spent in the black and/or white (Compartment 1 and 3) compartments, which were used to calculate the “preference score,” and these values expressed as a percentage of time in the two conditioning compartments; 2) Total number of beam breaks in each conditioning compartment; and 3) Preference score expressed as a percentage of time in the 2 conditioning compartments. Only preference scores for the CS+ expressed as a percentage of time in the 2 conditioning compartments were reported. A CPP was determined when the test subject spent more time in the test sample-paired compartment versus the vehicle-paired compartment. Conversely, a conditioned place aversion (CPA) was determined when the test subject spent more time in the vehicle-paired compartment versus the sample-paired compartment. Therefore, possible outcomes included whether AME-1 causes a dependence, an aversive effect, or no effect. The total time spent in the nonpreferred compartment+total time spent in both conditioning compartments was expressed as a percentage, and used as a measure of “side preference.” Side preference infers a hedonic valence for the environmental cues of the compartment. Initial side preferences/biases (Habituated Score-H) were used to determine which of two compartments would be paired with the targeted interoceptive states. After two CS-UCS pairings, groups were retested for side preference (Test 1-1; Test 2-2). A significant change score from the initial habituation test scores reflected a conditioned place approach (* p<0.05).

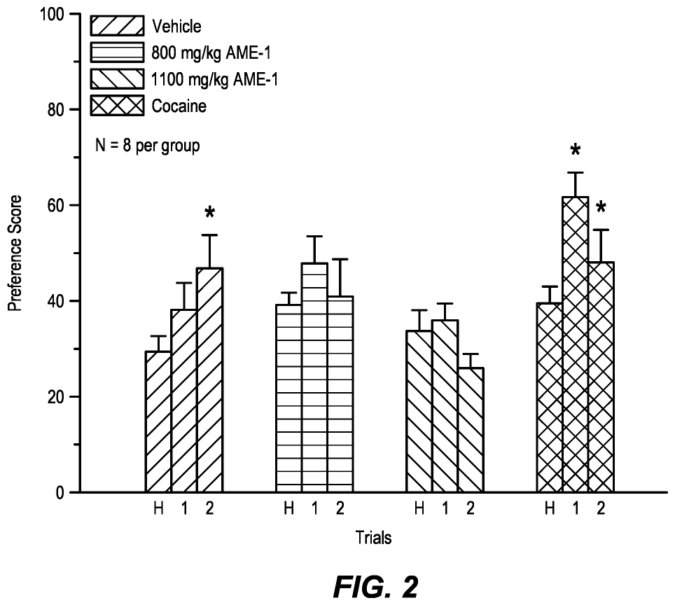

Results and Significance: FIG. 2 shows mean preference scores for different groups of rats administered 800 mg/kg AME-1, 1100 mg/kg AME-1, saline (negative control), or cocaine (positive control), prior to (H) and after (1, 2) place conditioning. Exemplary A. muscaria extract AME-1 did not produce CPP, i.e., dependence, or CPA, i.e., aversive effects, at the tested doses.

In contrast, cocaine did produce a CPP, while the initially preferred compartment in the negative-control group was diminished over repeated exposures to both chambers over two consecutive weeks of exposures with tap water.

Additionally, no apparent negative effects of AME-1, such as body weight fluctuations, were observed throughout the course of the study (data not shown). A summary of the mean preference scores for different treatment groups is shown in Table 4.

TABLE 4
Summary of Conditioned Place Preference Scores

| | | Timepoint | | | |
|---|---|---|---|---|---|
| Sex: Male | | Habituation | Test 1 | Test 2 | Overall |
| 0 mg/kg (Saline) | Mean | 29.440 | 38.325 | 46.911 | 38.225 |
| | SD | 8.7864 | 15.2870 | 19.3260 | 16.1673 |
| | N | 8 | 8 | 8 | 24 |
| | LSMean | 28.775 | 37.127 | 46.663 | 37.232 |
| | LSM s.e. | 4.2390 | 4.8683 | 5.0923 | 2.8053 |
| 800 mg/kg | Mean | 39.264 | 47.893 | 41.166 | 42.774 |
| A. muscaria | SD | 6.7157 | 15.8369 | 21.1666 | 15.5149 |
| extract | N | 8 | 8 | 8 | 24 |
| | LSMean | 39.014 | 47.641 | 40.644 | 42.394 |
| | LSM s.e. | 4.8581 | 5.0944 | 4.9547 | 2.8862 |
| 1100 mg/kg | Mean | 33.730 | 36.021 | 26.260 | 32.004 |
| A. muscaria | SD | 12.2771 | 9.3939 | 7.2180 | 10.3302 |
| extract | N | 8 | 8 | 8 | 24 |
| | LSMean | 32.768 | 35.482 | 25.680 | 31.150^{c} |
| | LSM s.e. | 4.5660 | 4.6942 | 3.9660 | 2.5709 |
| 20 mg/kg Cocaine | Mean | 39.593 | 61.194 | 48.208 | 49.665 |
| | SD | 9.6463 | 12.8426 | 16.7185 | 15.6794 |
| | N | 8 | 8 | 8 | 24 |
| | LSMean | 39.162 | 61.877 | 47.822 | 49.639^{a, f} |
| | LSM s.e. | 4.8723 | 4.8414 | 5.1009 | 2.9499 |
| Overall | Mean | 35.507 | 45.858 | 40.636 | |
| | SD | 10.0462 | 16.3691 | 18.4051 | |
| | N | 32 | 32 | 32 | |
| | LSMean | 34.795 | 45.420^{B} | 39.758 | |
| | LSM s.e. | 2.3418 | 2.5482 | 2.4686 | |

N—Number of measures used to calculate mean
SD—Standard Deviation
LSMean—Least squares mean
LSM s.e.—Least squares mean standard error
^{a}different from Group 1, p < 0.05
^{c}different from Group 2, p < 0.05
^{f}different from Group 3, p < 0.05
^{B}different from Habituation, p < 0.01

It is known from early learning studies that animals naturally approach pleasurable (hedonically positive) stimuli and avoid aversive (hedonically negative) stimuli. The CPP-CPA assay relies on this innate approach-avoidance behavioral tendency of animals. In this assay, animals were explicitly exposed to distinctive environmental cues with administration of a drug. Over repeated pairings of the drug (unconditioned stimulus, UCS) with the environment, the pleasurable or hedonically positive stimulus properties of the drug were conditioned to the unique environment in which it was administered (conditioned stimulus, CS). The animals were later tested by being presented with the opportunity to spend time in the presence of the cues paired with the drug or in the presence of cues not paired with the drug (i.e., with saline). Animals prefer to spend time in environments paired with a number of known drugs of abuse which can be classified as “positive” reinforcers. The results are consistent with an absence of drug-dependent reinforcing behaviors associated with administration of AME-1.

### Example 47: Repeated Administration of Exemplary A. muscaria Extract AME-1 and Effects Thereof on Gut Microvilli in Rats

Purpose: To determine the effects of repeated dosing of exemplary A. muscaria extract AME-1 on Toll-like receptor 2 (TLR2) and dendritic-cell-associated C-type lectin 1 (Dectin-1). The Toll-like receptors are an important family within the innate immune system and play an important role in host-defense mechanisms against pathogens. Dectin-1 is a cell surface pathogen recognition receptor that recognizes soluble and/or particulate forms of β-1,3 or β-1,6-glucans and also plays an important role in the host-defense mechanism.

Methods: Exemplary A. muscaria extract AME-1 was administered once daily by oral gavage to Sprague Dawley rats for 14 consecutive days. Different doses of AME-1 were assessed, including 500 mg/kg, 800 mg/kg, and 1100 mg/kg b.w. Ultrapure water (vehicle) was administered over the course of the experiment to rats in the negative control group.

On day 15, animals were euthanized and the abdominal cavity was incised and exposed. The cecum was identified and the area where the ileum joined the cecum towards stomach (˜15 cm) was cut. After collection of small intestine (Jejunum, Ileum), a 10-20 ml syringe filled with normal saline was inserted in the anterior opening of the intestinal segment. With holding the syringe inside the intestinal segment, the plunger of the syringe was pushed gently to flush the contents of the intestinal segment so that the entire length of intestine was clear. The intestine was then flushed with modified Bouin's fixative (50% Ethanol: 5% Acetic acid in water in the ratio of 1:1). The collected small intestine samples were preserved in modified Bouin's fixative. Samples were then processed by embedding in paraffin and sectioning prior to immunostaining and immunohistochemistry analysis.

Immunohistochemistry analysis of TLR2 and Dectin-1 in intestine samples was performed by using Rabbit Anti-TLR2 Antibody (ab213676, Abcam) and Rabbit Anti-Dectin-1 Antibody (ab189968, Abcam) as primary antibodies. Biotin conjugated Goat Anti-rabbit IgG H&L (ab7089, Abcam) was used as a secondary antibody.

Rat ovary sections were used as a positive assay control for TLR2 and Dectin-1 expression as recommended by the antibody product sheet supplied by the manufacturer. Intestine sections without incubation with the primary antibody were used as the negative control. Formalin-fixed paraffin-embedded tissues were sectioned at 4-6 μm thickness using a rotary microtome (Leica RM2245) and the sections were mounted on microscopic slides coated with 3-Aminopropyltriethoxysilane (APES).

Tissue sections were deparaffinized by warming on flattening table (Leica) at 60° C. for 10 min followed by xylene treatment. Sections were rehydrated with ethyl alcohol at a gradient concentration of 100%, 90% and 80% respectively followed by a wash with TBS washing buffer. After rehydration, antigen retrieval was performed by incubating sections in citrate buffer (0.01 M, pH 6.0) at 90-95° C. for 20 min and the slides were then cooled to room temperature.

Tissue sections were then subjected to blocking of endogenous peroxidase activity by incubating sections in 3% H₂O₂ solution in methanol at room temperature for 10 min. Following a rinse with washing buffer (TBS) for 5 min, the slides were then subjected to non-specific site blocking by incubating with 10% goat serum in PBS (blocking solution) for 1 h.

The sections were incubated overnight with either Rabbit Anti-TLR2 antibody (1:100 dilution in 5% goat serum in PBS) or Rabbit Anti-Dectin-1 antibody (1:100 dilution in 5% goat serum in PBS) in a humidified chamber maintained at 4° C. After overnight incubation, sections were washed using TBS and excess washing buffer was blotted.

Goat Anti-Rabbit IgG H&L (Biotin) (ab7089, Abcam) secondary antibody at a dilution of 1:1000 in PBS was added to the sections and incubated at room temperature in a humidified chamber for 1 h. After a series of washes with TBS, sections were incubated with IHC Select® Streptavidin-TRP enzyme conjugate solution for 30 min. The sections were then washed with TBS. Post-washing, DAB substrate solution (0.05%) was added to the sections and incubated for 2-3 min. Following washing with TBS, tissue sections were counterstained by immersing in hematoxylin solution for <1 min. Slides were then rinsed in running tap water to remove excess stain. Sections were subjected to dehydration using 80%, 90% and 100% of ethyl alcohol. Slides were cleared in xylene 3 times prior to mounting with coverslips using DPX mounting media.

The stained and mounted slides were examined microscopically (Model: Carl Zeiss Axioscope A1) for expression of TLR2 and Dectin-1. Five males and five females per group were selected for analysis. Three images per animal were captured at high power (400×) magnification and were further analyzed by Image J software for quantification of protein expression. Each image was analyzed under RGB mode with adjusting the color threshold in order to determine the percent area (% area).

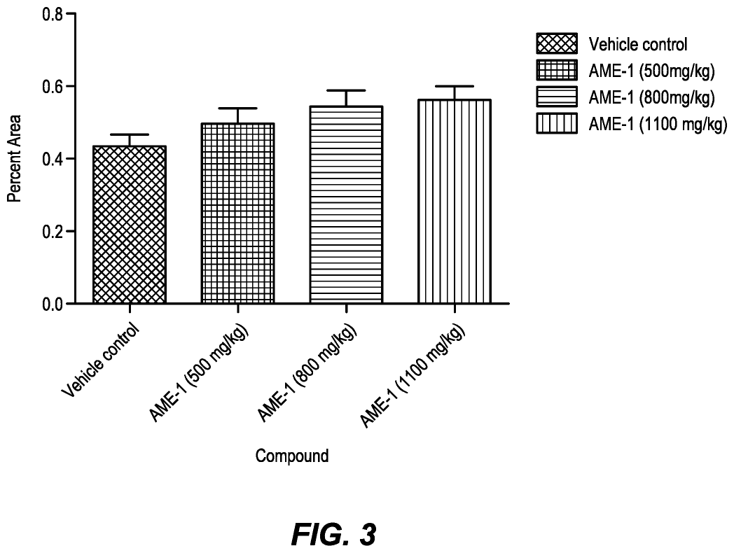

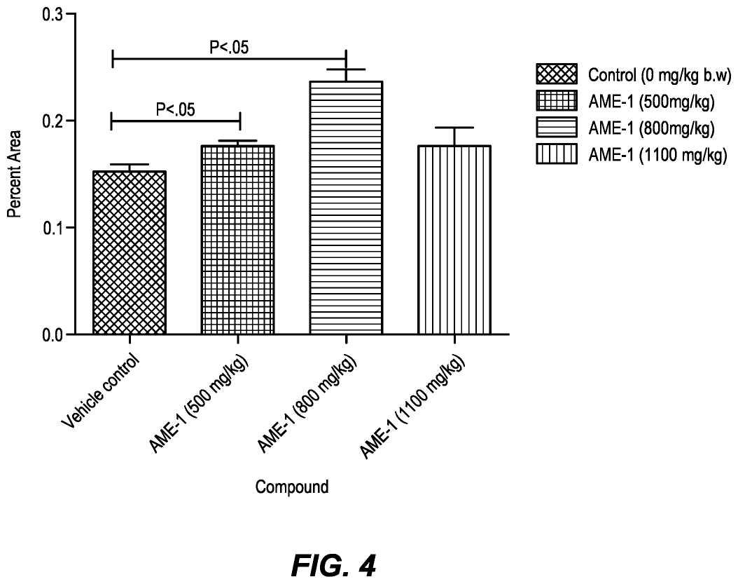

Results and Significance: FIG. 3 and FIG. 4 show the expression of Dectin-1 and TLR2 in rat gut microvilli following repeated dosing of different levels of AME-1 in comparison to a negative control. Whereas repeated exposure to AME-1 at all tested levels did not cause a significant change in Dectin-1 levels, exposure to AME-1 was associated with an increase in TLR2 expression.

The 14-day in vivo study resulted in a dose dependent increase in TLR-2 expression. This marginal increase of up to 2-fold indicates the potential of AME-1 to act as a potential bioactive agent, exerting a potential immune priming effect. Such priming effect may be mediated by the ability of AME-1 to increase cell surface plasma membrane concentration content of TLR-2. Dectin-1 under the same study conditions did not have any change in cell surface expression.

The in vivo study via oral supplementation with AME-1 focused on the potential bioactive effects on the immune receptors TLR-2 and Dectin-1, which are both found in gut epithelial cells. The in vivo results indicate that administration of AME-1 results in innate immune priming biological activity. The potential priming effect, resulting in a more efficient response to an invading pathogen thus promoting greater gut health and increased health and wellness of the individual.

### Example 48: High Performance Liquid Chromatography Performed on A. muscaria Extract Samples for Dose Confirmation Analysis

Purpose: The objective of the study was to report the dose confirmation analysis of the A. muscaria extract test samples. The study was conducted to determine if the muscimol content in A. muscaria extract test samples was within the acceptable limits using the validated method of extraction.

Methods: HPLC is used to separate, identify, and quantify components in a mixture. Briefly, a pressurized liquid solvent is passed through a column filled with solid adsorbent material. Each component interacts with the material differently, eliciting different flow rates and separating the components as they flow out of the column.

Mobile Phase A is prepared by combining 5 mM Ammonium formate in ultrapure water Type-I, mixing, sonicating, and storing at room temperature. Mobile phase B is prepared by transferring 1000 mL of acetonitrile into a 1000 mL schott bottle and storing at room temperature. Then, a diluent solution is prepared by combining 300 mL acetonitrile and 700 mL ultrapure water Type-I in a schott bottle, mixing, sonicating, and storing at room temperature. Finally, a rinsing solution is prepared by combining 500 mL of acetonitrile and 500 mL of ultrapure water Type-I in a schott bottle, mixing, sonicating, and storing at room temperature.

In a 5 mL volumetric flask, the reference standard stock solution containing muscimol is prepared by dissolving 5.005 g of muscimol reference standard in 3 mL of 30% acetonitrile (diluent solution). Then, the solution volume is made up to the mark using 30% acetonitrile, vortexed, and labeled with a final concentration of 1001.000 μg/mL. An additional standard containing 5.000 g of muscimol is prepared in the same manner for a final concentration of 1000.000 μg/mL.

Using a Gemini® 5 μm NX-C18 110 A° Column, 10 uL of the muscimol reference standard was injected with a flow rate of 0.8 mL/min. Then, the resulting data was processed using Lab Solution software 6.86 SP2.

Results and Significance: The system suitability test results met with acceptance criteria. The % CV of retention time for Muscimol was 0.00 and the % CV of area for Muscimol was 0.04 on 24/08/2021. The % CV of retention time for Muscimol was 0.20 and the % CV of area for Muscimol was 0.02 on 15/11/2021.

Homogeneity and Nonclinical Dose Formulation Analysis (24/08/2021) Homogeneity was evaluated for the low, mid and high dose concentrations of A. muscaria extract on 24/08/2021. Top, mid and bottom were prepared for each dose and homogeneity was evaluated. For low dose top homogeneity samples of A. muscaria extract, the % recovery and % CV were 98.81% and 0.03% respectively.

For low dose mid homogeneity samples of A. muscaria extract, the % recovery and % CV were 98.91% and 0.05% respectively. For low dose bottom homogeneity samples of A. muscaria extract, the % recovery and % CV were 98.68% and 0.06% respectively.

For mid dose top homogeneity samples of A. muscaria extract, the % recovery and % CV were 98.37% and 0.03% respectively. For mid dose mid homogeneity samples of A. muscaria extract, the % recovery and % CV were 98.84% and 0.03% respectively. For mid dose bottom homogeneity samples of A. muscaria extract, the % recovery and % CV were 98.01% and 0.03% respectively.

For high dose top homogeneity samples of A. muscaria extract, the % recovery and % CV were 99.42% and 0.07% respectively. For high dose mid homogeneity samples of A. muscaria extract, the % recovery and % CV were 98.10% and 0.02% respectively. For high dose bottom homogeneity samples of A. muscaria extract, the % recovery and % CV were 98.27% and 0.03% respectively. The method proved that the low, mid and high dose (top, mid and bottom layers) of A. muscaria extract were homogenous.

The mean recovery for the top, mid and bottom layers of low dose, mid dose and high dose were to be found 98.80%, 98.41% and 98.60% respectively. The % CV was calculated from the individual recovery and was within the acceptance criteria of 10%. Mean recoveries of all layers were within the acceptance criteria of 100±15% of the nominal concentration.

Homogeneity and Nonclinical Dose Formulation Analysis (15/11/2021) Homogeneity was evaluated for the low, mid and high dose concentrations of A. muscaria extract on 15/11/2021. Top, mid and bottom were prepared for each dose and homogeneity was evaluated.

For low dose top homogeneity samples of A. muscaria extract, the % recovery and % CV were 100.28% and 0.09% respectively. For low dose mid homogeneity samples of A. muscaria extract, the % recovery and % CV were 100.21% and 0.05% respectively. For low dose bottom homogeneity samples of A. muscaria extract, the % recovery and % CV were 100.39% and 0.12% respectively.

For mid dose top homogeneity samples of A. muscaria extract, the % recovery and % CV were 100.33% and 0.13% respectively. For mid dose mid homogeneity samples of A. muscaria extract, the % recovery and % CV were 100.19% and 0.10% respectively. For mid dose bottom homogeneity samples of A. muscaria extract, the % recovery and % CV were 100.20% and 0.13% respectively.

For high dose top homogeneity samples of A. muscaria extract, the % recovery and % CV were 99.57% and 0.13% respectively. For high dose mid homogeneity samples of A. muscaria extract, the % recovery and % CV were 99.48% and 0.07% respectively. For high dose bottom homogeneity samples of A. muscaria extract, the % recovery and % CV were 99.63% and 0.08% respectively.

The method proved that the low, mid and high dose (top, mid and bottom layers) of A. muscaria extract were homogenous. The mean recovery for the top, mid and bottom layers of low dose, mid dose and high dose were to be found 100.29%, 100.24% and 99.56%, respectively. The % CV was calculated from the individual recovery and was within the acceptance criteria of 10%. Mean recoveries of all layers were within the acceptance criteria of 100±15% of the nominal concentration.

The test sample concentrations for the dose confirmation analysis of A. muscaria extract were found to be within the acceptable limits and the prepared dose samples were consequently used for the toxicology studies of A. muscaria extract.

### Example 49: Use of Exemplary A. muscaria Extract AME-1 as a Supplement and Inclusion of the Same in Food and Beverage Products

Exemplary aqueous A. muscaria extract AME-1 comprises muscimol in an amount of 2.8% w/w, muscarine in an amount of 0.05% w/w, and ibotenic acid in an amount of 0.0015% w/w. AME-1 is used as a food ingredient in beverage powders and ready-to-drink beverages for conventional food use as well as a dietary ingredient in dietary supplements. The maximum amount (in milligrams) per serving is 25 mg/serving (containing 0.7125 mg/serving of an alkaloid blend) for ready-to-drink beverages and 30 mg/serving (containing 0.855 mg/serving of of an alkaloid blend) for beverage powders.

AME-1 is intended for inclusion in dietary supplements by the intended target population at a maximum daily serving of 880 mg/person/day (containing 25.08 mg/person/day of an alkaloid blend). AME-1 may also be added to a variety of conventional food products, including beverage powders and ready-to-drink beverages, at a maximum total daily serving size of 872.718 mg/person/day. AME-1 comprises 24.87 mg/person/day of an alkaloid blend, AME-1 is appropriate for use by the general adult population, such as individuals of 18 years of age or older, with the exception of pregnant women and lactating mothers.

### Example 50: Administration of a Tincture Comprising an A. muscaria Extract to an Individual to Enhance Calmness

An individual is administered or self-administers in a single day less than 880 mg of an A. muscaria extract formulated as a tincture. The tincture is administered by placing a drop of the A. muscaria composition directly under the tongue and holding it there for a duration of between about 1 to about 30 seconds, wherein the range is inclusive. The composition may then be swallowed by the individual.

Post-administration, the individual experiences enhanced calmness compared to the level of calmness experienced prior to administration, as measured by, e.g., an assessment completed by the individual prior to, and after administration.

### Example 51: Administration of a Dietary Supplement Comprising an A. muscaria Extract to an Individual to Enhance Calmness

An individual is administered or self-administers in a single day less than 880 mg of A. muscaria formulated as a dietary supplement which may be, for example, a chewable tablet, a capsule, a tablet, a lozenge, or a pill, with exemplary formulations provided in the Examples. To administer the dietary supplement, the supplement is administered to an individual or self-administered and is then either chewed and swallowed, or swallowed without chewing.

Post-administration, the individual experiences enhanced calmness compared to the level of calmness experienced prior to administration, as measured by, e.g., an assessment completed by the individual prior to, and after administration.

### Example 52: Topical Administration of an A. muscaria Composition to an Individual to Reduce Muscle Tension

An individual is administered or self-administers in a single day less than 880 mg of an A. muscaria composition formulated for topical application such as an ointment, salve, gel, paste, lotion, liniment, oil, patch, and/or cream, with exemplary formulations provided in the Examples. The topical formulation is administered by applying it directly onto an area experiencing muscle tension, or by applying it to, for example, one's hands and then rubbing it onto the area experiencing muscle tension. One may then optionally massage the affected area.

Post-application, the individual experiences reduced muscle tension compared to the level of muscle tension experienced prior to application of the topical formulation, as measured by, e.g., an assessment completed by the individual prior to, and after such application. As an example, an individual may rate their muscle tension on a scale of 0-10 prior to, and after applying the topical formulation, wherein “0” represents an absence of muscle tension, and “10” represents severe muscle tension that restricts movement. In such an example, application of the topical formulation will result in a reduced muscle tension rating.

As another example, an individual may measure their range of motion prior to, and after applying the topical formulation. In such an example, the individual will experience increased range of motion after applying the topical formulation.

### Example 53: Administration of a Beverage Comprising an A. muscaria Extract to an Individual to Reduce Insomnia

An individual is administered or self-administers in a single day about 880 mg of an A. muscaria extract formulated as a beverage powder, or a ready to drink beverage, with exemplary formulations provided in the Examples. The beverage powder may be administered by admixing the beverage powder and an ingestible liquid, for example water, milk, juice, tea, lemonade, limeade, and/or a carbonated beverage, such as a soda; and then ingesting the ingestible liquid admixed with the beverage powder. Likewise, the ready to make beverage may be formulated as any of the ingestible liquids disclosed above.

Post-ingestion, the individual experiences reduced insomnia compared to the severity of insomnia prior to ingesting the beverage powder or ready to drink beverage. For example, the individual will experience the ability to, for example, fall asleep faster, stay asleep longer once asleep, and awake feeling more restful. This may be measured by, for example, a sleep assessment completed without ingesting, and after ingesting the beverage powder or ready to drink beverage. Example assessments include, for example, the insomnia severity index. In such an example, the Insomnia Severity Index (Insomnia severity index—ons.org) will decrease in score after ingesting the A. muscaria composition.

The above examples are illustrative only, and should not be construed as limiting the invention in any way. An A. muscaria composition formulated as any of Examples 1-44 may be administered to an individual or is self-administered by an individual seeking to enhance calmness, alleviate muscle tension, and/or reduce insomnia, and may use assessments such as those described above to demonstrate the effectiveness of such administration.

The foregoing description, for purposes of explanation, uses specific nomenclature to provide a thorough understanding of the invention. However, it will be apparent to one skilled in the art that specific details are not required in order to practice the invention. Thus, the foregoing description of specific embodiments of the invention is presented for purposes of illustration and description. It is not intended to be exhaustive or to limit the invention to the precise forms disclosed; obviously, many modifications and variations are possible in view of the above teachings. The embodiments were chosen and described in order to best explain the principles of the invention and its practical applications, through the elucidation of specific examples, and to thereby enable others skilled in the art to best utilize the invention and various embodiments with various modifications as are suited to the particular use contemplated, when such uses are beyond the specific examples disclosed. Accordingly, the scope of the invention shall be defined solely by the following claims and their equivalents.

1. An Amanita muscaria extract, wherein the extract comprises a weight ratio of muscimol to ibotenic acid of at least 1000:1.

2. The extract of claim 1, wherein the extract comprises a weight ratio of muscimol to ibotenic acid of at least 1500:1.

3. The extract of claim 1, wherein the extract comprises a weight ratio of muscimol to ibotenic acid of about 1500:1, 1550:1, 1600:1, 1650:1, 1700:1, 1750:1, 1800:1, 1850:1, 1900:1, 1950:1, or 2000:1.

4. The extract of any of the preceding claims, wherein the extract comprises a weight ratio of muscimol to muscarine of at least 40:1.

5. The extract of any of the preceding claims, wherein the extract comprises a weight ratio of muscimol to muscarine of about 40:1, 45:1, 50:1, 55:1, 60:1, 65:1, or 70:1.

6. The extract of any of the preceding claims, wherein the extract comprises less than 30,000 μg/g of muscimol.

7. The extract of any of the preceding claims, wherein the extract comprises less than 550 μg/g of muscarine.

8. The extract of any of the preceding claims, wherein the extract comprises less than 20 μg/g of ibotenic acid.

9. The extract of any of the preceding claims, wherein the extract comprises less than 40 μg/g of stizolobinic acid.

10. A dietary supplement comprising the extract of any of the preceding claims.

11. A functional food comprising the extract of any of the preceding claims.

12. The functional food of claim 11, wherein the functional food is a food product.

13. The functional food of claim 11, wherein the functional food is a beverage.

14. The functional food of claim 13, wherein the beverage is a ready-to-drink beverage.

15. The functional food of claim 11, wherein the functional food is a beverage powder.

16. A pharmaceutical composition comprising the extract of any one of the preceding claims.

17. A compound of Formula (I): 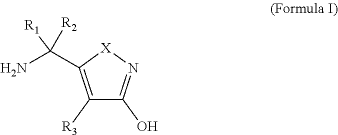
wherein R₁ is H or CH₃;
wherein R₂ is H, —CH₃, —CH₂CH₃, —CH₂CH₂CH₃, —CH(CH₃)₂, —CH(CH₃)(CH₂CH₃), —CH₂CH(CH₃)₂, —C(O)(OCH₃), —C(O)(OCH₂CH₃), —CH₂CH₂SCH₃, phenyl, or benzyl;
wherein R₃ is H, —CH₃, —CH₂CH₃, —CH₂CH₃, —CH₂CH₂OH, —CH₂CH₂Cl, F, Br, I, —CF₃, or —CN; and
wherein X is O or S;
or a pharmaceutically acceptable salt, prodrug, hydrate, or solvate thereof.

18. A compound of Formula (II): 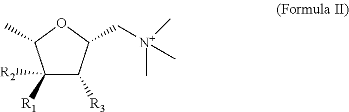
wherein R₁ is H, OH, F, O-benzyl, or O-benzoyl;
wherein R₂ is H or OH; and
wherein R₃ is H, OH, F, or phenyl;
or a pharmaceutically acceptable salt, prodrug, hydrate, or solvate thereof.

19. A pharmaceutical composition comprising a compound of Formula (I), a compound of Formula (II), or a compound of Formula (I) and a compound of Formula (II).

20. The pharmaceutical composition of claim 20, having a compound of Formula (I) and a compound of Formula (II), wherein the compound of Formula (I) and the compound of Formula (II) are present in a weight ratio of at least 40:1.

21. The pharmaceutical composition of claim 19 or claim 20, having a compound of Formula (I) and a compound of Formula (II), wherein the compound of Formula (I) and the compound of Formula (II) are present in a weight ratio of about 40:1, 45:1, 50:1, 55:1, 60:1, 65:1, or 70:1.

22. The pharmaceutical composition of any of claims 19 to 21, wherein the composition further comprises a weight ratio of muscimol to ibotenic acid of at least 1000:1.

23. The pharmaceutical composition of any of claims 19 to 22, wherein the composition does not comprise stizolobinic acid.

24. The pharmaceutical composition of any of claims 19 to 23, further comprising a pharmaceutically acceptable carrier, diluent, or excipient.

25. The extract, compound, or pharmaceutical composition of any of the preceding claims, wherein the extract, compound, or pharmaceutical composition is suitable for oral, sublingual, buccal, mucosal, injectable, intranasal, inhalation, or transdermal administration.

26. The extract, compound, or pharmaceutical composition of any of the preceding claims, wherein the extract, compound, or pharmaceutical composition is prepared as a liquid suspension, a liquid solution, a tincture, a tablet, a capsule, an intravenous solution, an injectable form, a topical form, a transdermal delivery form, a cut matrix sublingual or buccal tablet, a sublingual or buccal lozenge, a confectionery product, a vaporizer formulation, an intranasal delivery form, a beverage concentrate or beverage, a syrup, an elixir, an oral thin film, a troche, a caplet, a functional food, an effervescent powder, an herbal or medicinal tea, a nutritional supplement, a nutritional softgel supplement, a nutraceutical powder, and an infused chocolate.

27. The extract, compound, or pharmaceutical composition of any of the preceding claims in unit dosage form.

28. The extract, compound, or pharmaceutical composition of any of the preceding claims, wherein the extract, compound, or pharmaceutical composition is suitable for oral administration.

29. The extract, compound, or pharmaceutical composition of any of the preceding claims, wherein the extract, compound, or pharmaceutical composition is suitable for sublingual or buccal administration.

30. The extract, compound, or pharmaceutical composition of any of the preceding claims, wherein the extract, compound, or pharmaceutical composition is suitable for topical or transdermal administration.

31. The extract, compound, or pharmaceutical composition of any of the preceding claims, wherein the extract, compound, or pharmaceutical composition further comprises one or more additional active agents.

32. The extract, compound, or pharmaceutical composition of claim 31, wherein the one or more additional active agents is any of an agent useful in treating an anxiety disorder, an agent useful in treating a substance use disorder, an agent useful in treating pain or a pain disorder, an agent useful in treating an inflammatory disease or disorder, an agent useful in treating immune or autoimmune disorders, and a serotonergic agent.

33. A method of modulating neurotransmission comprising administering the extract, compound, or pharmaceutical composition of any of the preceding claims to a subject, thereby modulating neurotransmission in said subject.

34. The method of claim 33, wherein the neurotransmission is one or more of gabaminergic neurotransmission, glutaminergic neurotransmission, and cholinergic neurotransmission.

35. A method of treating a health condition, comprising administering to a patient an effective amount of the extract, compound, or pharmaceutical composition of any of the preceding claims.

36. The method of claim 35, wherein the health condition is a mental health disorder.

37. The method of claim 36, wherein the mental health disorder is selected from depression, dysthymia, an anxiety and phobia disorders, generalized anxiety disorder, social anxiety disorder, panic disorder, post-traumatic stress disorder, an adjustment disorders, a feeding and eating disorders, binge eating disorder, bulimia, and anorexia nervosa, other binge behaviors, body dysmorphic syndromes, alcoholism, tobacco abuse, drug abuse or dependence disorders, disruptive behavior disorders, impulse control disorders, gaming disorders, gambling disorders, memory loss, dementia of aging, attention deficit hyperactivity disorder, personality disorders, antisocial personality disorder, avoidant personality disorder, borderline personality disorder, histrionic personality disorder, narcissistic personality disorder, obsessive compulsive disorder, paranoid personality disorder, schizoid personality disorder, schizotypal personality disorders, attachment disorders, autism, and dissociative disorders.

38. The method of claim 36, wherein the mental health disorder is an anxiety disorder.

39. The method of claim 38, wherein the anxiety disorder is any of acute stress disorder, anxiety due to a medical condition, generalized anxiety disorder, panic disorder, panic attack, a phobia, post traumatic stress disorder (PTSD), separation anxiety disorder, social anxiety disorder, substance-induced anxiety disorder, and selective mutism.

40. The method of claim 36, wherein the mental health disorder is a substance use disorder.

41. The method of claim 40, wherein the substance use disorder is any of alcohol use disorder, Cannabis use disorder, hallucinogen use disorder, inhalant use disorder, opioid use disorder, sedative use disorder, stimulant use disorder, tobacco use disorder, and nicotine use disorder.

42. The method of claim 36, wherein the mental health disorder is a behavioral addiction.

43. The method of claim 42, wherein the behavioral addiction is selected from gambling disorder, gaming disorder, sexual addiction, compulsive buying disorder, and technology addiction.

44. The method of claim 35, wherein the health condition is a sleep disorder.

45. The method of claim 44, wherein the sleep disorder is any of an insomnia, a hypersomnia, a parasomnia, and a disorder of sleep-wake schedule.

46. The method of claim 35, wherein the health disorder is a physical health disorder.

47. The method of claim 46, wherein the physical health disorder is a pain disorder.

48. The method of claim 47, wherein the pain disorder is any of arthritis, allodynia, atypical trigeminal neuralgia, trigeminal neuralgia, somatoform disorder, hypoesthesis, hypealgesia, neuralgia, heuritis, neurogenic pain, analgesia, anesthesia dolorosa, causlagia, sciatic nerve pain disorder, degenerative joint disorder, fibromyalgia, visceral disease, chronic pain disorders, migraine/headache pain, chronic fatigue syndrome, complex regional pain syndrome, neurodystrophy, plantar fasciitis, or pain associated with cancer.

49. The method of claim 46, wherein the physical health disorder is a disorder that causes acute inflammation, or that exhibits chronic inflammation as a symptom.

50. The method of claim 46, wherein the physical health disorder is an autoimmune disorder.

51. The method of claim 50, wherein the autoimmune disorder is any of acute disseminated encephalomyelitis (ADEM), Addison disease, allergy or hypersensitivity, amyotrophic lateral sclerosis, antiphospholipid antibody syndrome (APS), arthritis, autoimmune hemolysis Anemia, autoimmune hepatitis, autoimmune inner ear disease, autoimmune pancreatitis, bullous pemphigoid, celiac disease, Chagas disease, chronic obstructive pulmonary disease (COPD), type 1 diabetes (T1D), endometriosis, fibromyalgia, goodpasture's syndrome, Graves' disease, Guillain-Barre syndrome (GBS), Hashimoto's thyroiditis, suppurative spondylitis, idiopathic thrombocytopenic purpura, inflammatory bowel disease, interstitial cystitis, lupus, including discoid lupus erythematosus, drug-induced lupus lupus erythematosus, lupus nephritis, neonatal lupus, subacute cutaneous lupus erythematosus, and systemic lupus erythematosus; morphea, multiple hard Keratosis (MS), myasthenia gravis, myopathy, narcolepsy, neuromuscular angina, pemphigus vulgaris, pernicious anemia, primary biliary cirrhosis, recurrent diffuse encephalomyelitis, including polyphasic diffuse encephalomyelitis, rheumatic fever, schizophrenia, scleroderma, Sjogren's syndrome, tendonitis, vasculitis, and vitiligo.

52. The method of claim 50, wherein the autoimmune disorder is a systemic autoimmune disorder, including systemic lupus erythematosus (SLE), Sjogren's syndrome, scleroderma, rheumatoid arthritis, and polymyositis.

53. The method of claim 50, wherein the autoimmune disorder is a local autoimmune disorder, including those of the endocrine system, including type 1 diabetes, Hashimoto's thyroiditis, and Addison's disease; the cutaneous, including pemphigus vulgaris; the blood, including autoimmune hemolytic anemia; and the nervous system, including multiple sclerosis.

54. A method of using the extract, compound, or composition of any of the preceding claims to improve health and wellness, comprising administering an effective amount of the extract, compound, or composition to a subject.

55. The method of claim 54, wherein the improvement in health and wellness is a reduction in stress.

56. The method of claim 54, wherein the improvement in health and wellness is an easing of muscular tension.

57. The method of claim 54, wherein the improvement to health and wellness is a promotion of restorative sleep.

58. The method of claim 54, wherein the improvement to health and wellness is any of a soothing of the body, a calming of the mind, and a reduction in physical distress.

59. The method of any one of claims 54 to 58, wherein the improvement to health and wellness includes any one or more of a reduction in feelings of nervousness, “jitters,” nervous tension, or anxiety; a reduction in feelings of malaise, unhappiness, existential angst, ennui, and general discontent; and an increase in feelings of wellbeing, wellness, relaxation, contentment, happiness, openness to experience, and life satisfaction.

60. A method of using the extract, compound, or composition of any of the preceding claims to induce euphoria, comprising administering an effective amount of the extract, compound, or composition to an individual.
